(19) United States
(12) Patent Application Publication
(10) Pub. No.: US 2008/0020379 A1
(43) Pub. Date: Jan. 24, 2008

(54) Diagnosis and prognosis of infectious diseases clinical phenotypes and other physiologic states using host gene expression biomarkers in blood

(76) Inventors: Brian K. Agan, San Antonio, TX (US); Eric H. Hanson, Las Vegas, NV (US); Michael J. Jenkins, San Antonio, TX (US); Baochuan Lin, Bethesda, MD (US); Chris C. Olsen, Cibilo, TX (US); Robb K. Rowley, Las Vegas, NV (US); David A. Stenger, Herndon, VA (US); Dzung C. Thach, Annandale, VA (US); Clark J. Tibbetts, Sperryville, VA (US); Elizabeth A. Walter, San Antonio, TX (US); Jinny Lin Liu, Ellicott, MD (US)

Correspondence Address: NAVAL RESEARCH LABORATORY;ASSOCIATE COUNSEL (PATENTS), CODE 1008.2, 4555 OVERLOOK AVE., SW, WASHINGTON, DC, 20375, US

(21) Appl. No.: 11/268,373

(22) Filed: Nov. 7, 2005

Related U.S. Application Data
(60) Provisional application No. 60/626,500, filed on Nov. 5, 2004.

(51) Int. Cl. C12Q 1/68

(52) U.S. Cl. 435/6

(57) ABSTRACT

The present invention provides a specific set of gene expression markers from peripheral blood leukocytes that are indicative of a host response to exposure, response, and recovery infectious pathogen infections. The present invention further provides methods for identifying the specific set of gene expression markers, methods of monitoring disease progression and treatment of infectious pathogen infections, methods of prognosing the onset of an infectious pathogen infection, and methods of diagnosing an infectious pathogen infection and identifying the pathogen involved.

### CROSS-REFERENCE TO RELATED APPLICATIONS

The present application claims priority to U.S. 60/626,500, filed on Nov. 5, 2004, the entire contents of which are incorporated by reference.

### STATEMENT REGARDING FEDERALLY FUNDED PROJECT

The United States Government owns rights in the present invention pursuant to funding from the Defense Threat Reduction Agency (DTRA; Interagency Cost Reimbursement Order (IACRO #02-4118), MIPR numbers 01-2817, 02-2292, 02-2219, and 02-2887), the Office of the U.S. Air Force Surgeon General (HQ USAF SGR; MIPR Numbers NMIPR035203650, NMIPRONMIPR03520388 1, NMIPRONMIPR035203881), the U.S. Army Medical Research Acquisition Activity (Contract # DAMD17-03-2-0089), the Defense Advance Research Projects Agency (DARPA; MIPR Number M189/02), and the Office of Naval Research (NRL Work Unit 6456).

### REFERENCE TO SEQUENCE LISTING

The present application includes a sequence listing on an accompanying compact disk containing a single file named “AED 764 (GXP) Sequence Listing,” created on Nov. 7, 2005 and 2 KB in size.

The entire contents of that accompanying compact disk are incorporated by reference into this application.

### REFERENCE TO TABLES

The present application includes 18 tables on an accompanying compact disk containing the following files:

| File Name | Format | Size | Created |
|---|---|---|---|
| Table 16.txt | MS Windows ASCII | 6 kb | Nov. 03, 2005 |
| Table 17.txt | MS Windows ASCII | 2 kb | Nov. 03, 2005 |
| Table 18.txt | MS Windows ASCII | 802 kb | Nov. 03, 2005 |
| Table 19.txt | MS Windows ASCII | 3 kb | Nov. 03, 2005 |
| Table 20.txt | MS Windows ASCII | 4 kb | Nov. 03, 2005 |
| Table 21.txt | MS Windows ASCII | 2 kb | Nov. 03, 2005 |
| Table 22.txt | MS Windows ASCII | 215 kb | Nov. 03, 2005 |
| Table 23.txt | MS Windows ASCII | 4 kb | Nov. 03, 2005 |
| Table 24.txt | MS Windows ASCII | 3 kb | Nov. 03, 2005 |
| Table 25.txt | MS Windows ASCII | 2 kb | Nov. 03, 2005 |
| Table 26.txt | MS Windows ASCII | 153 kb | Nov. 03, 2005 |
| Table 27.txt | MS Windows ASCII | 4 kb | Nov. 03, 2005 |
| Table 28.txt | MS Windows ASCII | 705 kb | Nov. 03, 2005 |
| Table 29.txt | MS Windows ASCII | 3 kb | Nov. 03, 2005 |
| Table 30.txt | MS Windows ASCII | 491 kb | Nov. 03, 2005 |
| Table 31.txt | MS Windows ASCII | 3 kb | Nov. 03, 2005 |
| Table 32.txt | MS Windows ASCII | 81 kb | Nov. 03, 2005 |
| Table 33.txt | MS Windows ASCII | 5 kb | Nov. 03, 2005 |

The entire contents of that accompanying compact disk are incorporated by reference into this application.

LENGTHY TABLES FILED ON CD
The patent application contains a lengthy table section. A copy of the table is available in electronic form from the USPTO web site (http://seqdata.uspto.gov/?pageRequest=docDetail&DocID=US20080020379A1). An electronic copy of the table will also be available from the USPTO upon request and payment of the fee set forth in 37 CFR 1.19(b)(3).

### BACKGROUND OF THE INVENTION

1. Field of the Invention

The present invention provides a specific set of gene expression markers from whole blood and/or peripheral blood leukocytes (PBL) that are indicative of a host response to exposure, response, and recovery from infectious pathogens. The present invention further provides methods for identifying the specific set of gene expression markers, methods of monitoring disease progression and treatment of infectious pathogen infections, methods of predicting the onset of the symptoms and/or manifestation of an infectious pathogen infection, and methods of diagnosing an infectious pathogen infection and classifying the pathogen involved.

The present invention also provides the following:
(1) methods for validating the differential gene expression markers in a cohort (such as a Basic Military Trainee (BMT) population). Such a method can be used to validate and/or expand upon a subset of biomarkers identified by alternative techniques for a specific disorder,
(2) methods for designing and implementing a process of determining pre-symptomatic gene expression changes in an exposed population,
(3) methods for statistical (e.g. Bayesian) inference to combine other (e.g. metadata) information into a overall diagnosis or assessment, and
(4) alternative measurement techniques other than Genechip microarrays, though not necessarily excluding Genechip microarray, that could be used to measure changes in a small, differentiating subset of genes (i.e., a subset of genes identified by the microarray-based method of the present invention) in a minimal volume of blood (lancet to produce drops of blood instead of intravenous blood draw to produce milliliters of blood) in a period of hours instead of days.

Moreover, the present invention relates to an overall business model, components of which include:
(1) assessment of the morbidity potential of individuals who were exposed to an infectious pathogen or agent of chembio-terrorism using pre-symptomatic gene expression markers,
(2) pre-assessment of the morbidity potential for select individuals (e.g. aircrews prior to the start of a 24 hour mission) or for general public use for pro-active intervention against infectious disease prior to the onset of major symptoms, and
(3) assessment of human behavioral activities (i.e. Exercising, eating, fasting, smoking, etc) that affect physiology and blood gene-expression, thus enabling discovery of biomarkers related to these behaviors that may be used to establish past activities of an individual at a certain probability of confidence.

The present invention further relates to:
(1) methods for extrapolating the methods developed herein (e.g., PAXgene processing and metadata) for use in other disease diagnostics (e.g., blood-related; autoimmune diseases, leukemia);
(2) methods for assembly of metadata in a format that allows it to be assimilated into inferential models of disease assessment; and
(3) methods for establishing a comprehensive human gene expression baseline database, against which perturbations, such a pathogen exposure, infection, and other disease states would be compared.

2. Discussion of the Background

Recent years have witnessed an explosive growth in the number of applications involving the use of DNA microarrays to monitor the expression of genes in various forms of tissues and cultured cells (1-5). Such “expression profiling” requires a measurable change in the relative abundance of transcribed messenger RNA (mRNA) in host cells in response to some type of perturbation. The measurement is usually performed indirectly by reverse transcription (RT) of the labile mRNA into more stable complementary DNA (cDNA) which is in turn labeled with a fluorophore (true for most work, but the-Affymetrix process involves re-conversion of cDNA back to RNA, which is in turn labeled and hybridized) and allowed to hybridize with the microarrays containing a plurality of DNA “probe” molecules that bind the target cDNA of interest.

Typically, colored fluorophores are used to label the “control” and “experimental” pools of cDNA, allowing the relative transcript abundances to be deduced from the ratio of fluorescence intensities. Alternatively, a single color measurement can be enabled by scaling of the intensities between different microarrays, as in the case with Affymetrix high-density microarrays (vide infra) because the variation from among Affymetrix arrays are minimal compared to most spotted array platforms. Defining sets of genes that are modulated in response to the external perturbation is non-trivial and is complicated by “noise” due to biologic variability, microarray production batch, handling factors, and variability emerging during sample processing (6).

Types of Microarray Probe Molecules

Significantly, the DNA probes themselves can be of highly variable lengths. Probes comprised of cDNA molecules (which are RT/PCR products of transcriptional isolates known as “Expressed Sequence Tags”; ESTs) can have varying lengths (usually hundreds of base pairs) and are often adsorbed (non-covalently) and then cross-linked (chemically or using ultraviolet radiation) to positively-charged poly-lysine or aminosilane-coated microscope slides. In contrast, probes comprised of defined “long” (70-mer) or “short” (25-mer) oligonucleotides are of fixed length and are almost invariably attached by a covalent bond via one terminus of the DNA molecule. Higher degrees of transcript detection sensitivity can usually be achieved with 70-mer probes compared to shorter ones (e.g. 20-25 mers). However, specificity is reduced because 70-mer target/probe hybridizations are generally insensitive to small numbers (e.g., 2-3) of single base mismatches, whereas shorter probes are sensitive to single mismatches and thus provide greater specificity. In contrast, little can be said about transcript-specific cDNA binding to complementary cDNA probes prepared from EST libraries, because the length of the probes (hundreds of base pairs) can result in binding of multiple smaller transcription-specific cDNA molecules. The separation of these contributions would be impossible from a single fluorescent intensity signal as measured by a microarray scanner.

At least a few research groups have developed microarrays that are capable of distinguishing varying levels of “sequence resolution”. Within the human genome, only a small percentage of the total sequences called “exons” actually encode for functional polypeptides and these segments are interspersed with non-coding segments called “introns”. Shoemaker et al (7) developed “exon arrays” comprised of long (50-60 bases) targeting predicted exon regions, and “tiling arrays” which used sets of similar length overlapping oligonucleotides to completely blanket a genomic region of interest for human chromosome 22. This allows for determination of most RNA transcripts from this chromosome, including transcripts that are not traditionally considered as genes. Additionally, these microarrays should also be able to locate mutations in the chromosomal DNA itself. Further, this allows determination of which exons are represented in the formation of specific splice variants of transcripts coding for functional proteins.

For the present invention, the authors have used Affymetrix HG-U133A and HG-U133B Human Genome Expression Chips (Part No. 900444; for detailed information refer to the product literature available from the manufacturer, which is hereby incorporated by reference in its entirety) as well as the HG-U133 plus 2.0 chip (Part No. 900467) which contains probes from HG-U133A, HG-U133B, and an additional 10,000 probeset on one cartridge. A GeneChip® probe array contains “cells”, each having a large number of copies of a unique 25-mer probe and arranged in probe pairs consisting of a perfect match (PM) and a mis-match (MM) wherein the middle (number 13) position is varied. Normally, RNA is extracted from samples and reverse transcribed into cDNA then into double stranded cDNA with a T7 promoter region added. Then in vitro transcription is carried out to linearly amplify the RNA and incorporate biotinylated nucleotides to make biotin labeled cRNA. The labeled cRNA target is hybridized onto the microarray, usually over night, then follow by washing and detection via strepavidin conjugated fluorescent dyes the next day. Following hybridization of the labeled transcriptional targets to the microarray (for detailed information refer to the product literature available from Affymetrix entitled ‘Eukaryotic Sample and Array Processing,’ which is hereby incorporated by reference in its entirety), the Affymetrix GCOS software (manual available from Affymetrix) (8) is used to reduce the raw scanned image (.DAT) file to a simplified file format (.CEL file) with intensities assigned to each of the corresponding probe positions.

A graphical description of the probe pair layouts and the expression analysis algorithm is found in the Affymetrix GCOS manual on pages 505-523 (8). On the U133A and B GeneChips®, each (˜39,000) known and putative gene from the Unigene database U133 build of the human genome (for detailed information refer to the product literature available from the manufacturer, which is incorporated by reference in its entirety) are represented by probe pairs spaced across some length of the gene, with some bias towards the 3′ end (maps and analysis available through the NetAffx website available through the Affymetrix website). The GCOS software executes algorithms to assign an overall intensity that is used to infer abundance of a transcript and calculate fold changes of expression between two or more experiments. It also provides a metric to indicate whether a gene is “present” (detectably expressed) or absent. Following these calculations, the individual probe intensities are not explicitly referenced but they remain part of the permanent data in the .CEL file for each experiment.

Thus, there are considerable differences in the interpretability of “gene expression” measurements, depending on the types and numbers of microarray probes used and the algorithms used to analyze the spatial patterns of intensity from the probes.

Transcriptional Markers

Of equal significance, relative to the “sequence resolution” of the measurement of transcript abundance in metazoan systems is the variation in the composition of “genes” and transcriptional gene products. Initial drafts of the human genome (9, 10) indicate that the human genome is comprised of approximately 30,000 genes, mostly identified by computational methods having significant limitations (11). Yet, orders of magnitude greater numbers of different proteins can be produced from these genes through the recombination of the internal coding sequences (exons) that are interspersed with non-coding sequences (introns). Hence, probes comprised of cDNA clones derived from a transcriptional library are biased towards detection of the complete gene product sequences that are obtained under a specific set of times and conditions, and cannot represent the multiform nature of mammalian gene expression in more general conditions where alternative splice variants will change the transcriptional sequence composition.

Prior Art in Gene Expression Profiling in the Immune Response to Pathogens

Cell Culture Models

Several groups have also measured the gene expression profiles of individual immune cell types following exposure to microbes or microbial components in vitro. Groups at Whitehead Institute (12) and Stanford (13) have used Affymetrix and spotted cDNA microarray types, respectively, to observe relatively stereotyped responses of cultured human peripheral blood mononuclear cells (PBMCs; i.e. circulating macrophage precursor cells, T lymphocytes, B lymphocytes), eosinophils, and basophils when exposed to a variety of killed bacteria and bacterial cell wall components. The similarity of the responses is reflective of evolutionarily conserved pro-inflammatory responses within the innate immune system and do not suggest that pathogen-specific responses would be obviously detectable. Chaussable et al (14) describe a study with in vitro generated macrophages and dendritic cells, which provides insights into the innate immune response to diverse pathogens but is impractical for surveillance, as these cells types can only be isolated by laboratory procedures that will change their natural gene expression.

Peripheral Blood Leukocytes (PBLs) Drawn from the Infected Host

Craig Cummings, David Relman and Patrick Brown (Stanford University) hypothesized that the unique mixtures of virulence factors expressed by specific pathogens will give rise to a correspondingly unique transcriptional response in the host (15). They reasoned that an attractive host tissue source would be peripheral blood leukocytes (PBLs) because any pathogen gaining access to the body will elicit a multiplicity of immune response mechanisms, each characterized by combinations of specific gene modulations. They also pointed out that this technique might allow early diagnosis of even uncultivable or uncharacterized pathogens, that variations in host expression profiles could allow inference of time since exposure, and that a single technique could be used to diagnose a large number of different diseases.

Relman et al have used variations of the “Lymphochip” (16, 17) (which is comprised of probes for approximately 3,000-3,500 “lymphoid” genes comprised of cDNA clones prepared from transcriptional libraries of human lymphoid tissues) to analyze expression changes in cultured PBMCs (13), and in PBLs (PBL contributions—all white blood cells and the differential is typically 41-77% neutrophils, 20-51% lymphocytes, 1.7-9% monocytes and less than one percent of basophils and eosinophils), from RNA isolated from PAXgene Blood RNA tubes from 75 healthy human donors (18). The latter study (18) illustrated that relative gene expression levels in PBLs are related to variations in specific blood cell types, gender, age, and time of day. Relman et al have also observed changes in PBMC expression in non-human primates (NHPs) following experimental inoculation with Variola major, the virus responsible for human smallpox. In addition, Relman et al compared Ebola infection of NHP. However, the inventors herein are unaware of any disclosures that relate those changes to NHP inoculations using other pathogens or to baseline gene expression in humans. Because of the type of microarray (cDNA EST clones) it is not possible to ascribe particular transcriptional sequences that are responsible for assigning fold changes to particular genes. The present inventors are unaware of any written descriptions existing in the public domain that describe these data.

In short, all of Relman's papers use cDNA arrays and PBMCs (which require on site isolation centrifuge and technicians). If they used paxgene, they processed it within 24 hours. This is not practical for surveillance. Whereas in the present invention, the inventors demonstrate that the paxgene tubes can give decent gene expression profiles even when handled in conditions amendable to surveillance. Relman did not know and/or test this; hence they did everything within 24 hours to be safe in the notation that the RNA has not degraded. Also, for cDNA arrays, Relman required reference RNA with gene expression profiles similar to tissue of interest to compare 2 colors for all chips, which makes it impractical to study large population expressing different genes than what is contained within their reference RNA. Whereas the Affy chip is single color so no reference common RNA is needed allowing us to compare large numbers of chips overtime, especially when we spike in normalization control RNA.

Differential Gene and Protein Expression Following Exposure to Biological Warfare Agents

At least one U.S. Pat. No. 6,316,197 B1 (19) makes claim to methods for determining characteristic gene expression changes from an infected host to diagnose exposure to biological warfare (or bioterrorism) agents. The inventors of that application described a series of steps that begin with the use of differential display PCR (DD-PCR) to discover genes that are expressed differently in cultured cells following incubation with biological toxins (e.g. Staphyloccocus enterotoxin B; SEB, and Botulinum toxin) or microbes (e.g. Bacillus anthracis). Briefly, DD-PCR involves the use of reverse transcriptase to convert host RNA transcripts to cDNAs, which are in turn amplified with PCR and separated by gel electrophoresis. Specific sequences are determined for each of the corresponding electrophoretic bands to identify the differentially expressed genes. The inventors of U.S. Pat. No. 6,316,197 described methods for measuring (including the use of reverse transcriptase PCR and DNA microarray hybridization) correlating the observed changes with methods for measurement in animals exposed to the same agents, and found gene expression changes that corresponded to those observed in culture. Overall, this work makes use of a commonly used method of discovering genes that are involved in differential biological responses and implicates several transcriptional markers that correlate with the exposure to several types of toxic insult. However, there is no ethical way to perform the same experiments using humans, and consequently, no manner of obtaining clinically relevant data for a human population. Nor is there an attempt in this work to compare the perturbations to a baseline human expression profile. Also, none of the methods disclosed by Relman et al are amendable to a surveillance setting

Differential Gene Expression Measurement in an Integrated Biodefense System

The concept of a microarray used for broad-spectrum pathogen identification has considerable and obvious appeal to both medical practice and national defense. This was best illustrated in the recommendations of the Defense Sciences Board (DSB) Summer 2000 Panel, which made recommendations to the DATSD (ATL) that the U.S. Defense Department develop a “Zebra Chip”; that is, a hypothetical microarray of unspecified technology that could include gene expression markers, that would be in widely distributed use (DoD TriCare System) as a routine clinical diagnostic for both common and uncommon (e.g. bioterrorism) infectious agents. In addition to having probes for common infectious agents, the Zebra Chip would also contain a large number of probes for unusual (“zebra”) pathogens. If such a device were in widespread use at the time of a biological terrorism event or a natural epidemic (e.g. SARS), the cost savings, both financial and in human suffering, could be enormous, due to the earliest possible detection of the agent when only minor (flu-like) symptoms were manifest.

Furthermore, there is a need to unambiguously define “baseline” expression profiles, against which the “perturbed” state profiles are compared, as they may be variable in time and between individuals.

Because it may not always be possible to identify the specific cause of an infection through pathogen genomic markers (e.g. using PCR or microarrays), there remains a critical need to determine alternative “biomarkers' from the host that would elucidate the character of the disease etiology and guide the clinician in the proper management of the infection.

Heretofore, none of the published prior art methods are amendable to large long-term field studies/surveillance. All of the published methods are simply for a quick one-time gene expression study. Therefore, and in view of the foregoing, there remains a critical need of methods for determining characteristics gene expression changes that arise from an infected host to diagnose disease states, help guide treatment regimens, and assist in making treatment/operational decisions. Further, there exists a critical need for rapid, near real-time methods useful for field implementation that may be used individually or in combination with additional detection and diagnostic methods and apparatuses.

### SUMMARY OF THE INVENTION

It is an object of the present invention to provide methods for determining the baseline gene expression in a healthy individual, as well as systematic changes in the gene expression pattern characteristic to a pathogen or infection. More specifically, this object relates to methods for establishing a comprehensive human gene expression baseline database, against which perturbations, such a pathogen exposure, infection, and other disease states would be compared.

It is another object of the present invention to provide a method for validating the differential gene expression markers identified in a cohort.

It is yet another object of the present invention to design and implement a process to determine pre-symptomatic gene expression changes in an exposed population and from this to design/tailor therapeutic regimens.

Within the aforementioned objects, the present invention further provides methods for statistical (e.g. Bayesian) inference to combine other (e.g. metadata) information into an overall diagnosis or assessment.

The objects of the present invention may be extended to and the present invention embraces extrapolating the methods developed herein (e.g., PAXgene processing and metadata) for use in other disease diagnostics.

Further, it is an object of the present invention to provide a method for assembly of metadata in a format that allows it to be assimilated into inferential models of disease assessment.

It is an object of the present invention to further an overall business model, which includes:
(1) assessment of the morbidity potential of individuals who were exposed to an infectious pathogen or agent of chembio-terrorism using pre-symptomatic gene expression markers,
(2) pre-assessment of the morbidity potential for select individuals (e.g. aircrews prior to the start of a 24 hour mission) or for general public use for pro-active intervention against infectious disease prior to the onset of major symptoms, and
(3) assessment of human behavioral activities (i.e., Exercising, eating, fasting, smoking, etc.) that affect physiology and blood gene-expression, thus enabling discovery of biomarkers related to these behaviors that may be used to establish past activities of an individual at a certain probability of confidence.
(4) banking of samples (i.e. Paxgene) in conjunction with clinical information database for any phenotype of interest now or in the future.

In a certain object of the present invention is to provide a method for determining the gene expression profile for (i) a healthy person and/or (ii) a subject that has been exposed to one or more infectious pathogens by
a) collecting a biological sample (e.g., whole blood) from a subject;
b) isolating RNA from said sample;
c) removing DNA contaminants from said sample;
d) spiking into said sample a normalization control;
e) synthesizing cDNA from the RNA contained in said sample;
f) in vitro transcribing cRNA from said cDNA and labeling said cRNA;
g) hybridizing said cRNA to a gene chip followed by washing, staining, and scanning; and
h) acquiring a gene expression profile from said gene chip and analyzing the gene expression profile represented by the RNA in said sample on the basis of (i) the health of the subject or (ii) the disease(s) said subject has been exposed to while controlling for confounder variables.

Within this object, the following additional steps may also be performed to increase the overall sensitivity of the method and to enhance the reliability of the results obtained thereby:
concentrating and purifying said RNA between (c) and (d);
reducing and/or eliminating globin mRNA in said sample between (d) and (e), for example adding biotinylated globin capture oligos to said sample to bind the globin mRNA and removing the resulting bound globin mRNA by strepavidin magnetic beads leaving globinclear RNA and, optionally, further-purifying the globinclear RNA by contacting said globinclear RNA with magnetic RNA binding beads or RNA binding column;
reducing and/or eliminating globin mRNA in said sample, coincident with (e), by adding PNA to said sample during said synthesizing cDNA; and/or
repeating (g) with a second gene chip, between (g) and (h), which is distinct from said gene chip in (g), wherein in (h) following acquisition the data obtained from said first and second gene chips is merged.

In another object of the present invention, is a method for identifying gene expression markers for distinguishing between healthy, febrile, or convalescence in subjects that have been exposed to one or more infectious pathogens by:
a) acquiring a gene expression profile by the method according to the aforementioned object for a subject that has been exposed to one or more infectious pathogens;
b) acquiring a gene expression profile by the method according to the aforementioned object for a subject that has recovered from exposure to said one or more infectious pathogens;
c) acquiring a gene expression profile by the method according to the aforementioned object for a healthy subject that has not been exposes to those one or more infectious pathogens;
d) comparing the gene expression profiles for the subjects from (a), (b), and (c) by a pairwise comparison;
e) determining the identify of the minimal set of genes that classify the patient phenotype as healthy, febrile, or convalescent by class prediction algorithm based on said pairwise comparison; and
f) assigning the classification of healthy, febrile, or convalescent and/or classifying adenovirus febrile infection from background cases of other febrile illness in the cohort based on gene expression profile of the minimal set of genes determined in (e).

In yet another object of the present invention, is a method of classifying a subject in need thereof as healthy, febrile, or convalescence, by
a) collecting a biological sample (e.g., whole blood) from said subject;
b) isolating RNA from said sample;
c) removing DNA contaminants from said sample;
d) spiking into said sample a normalization control;
e) synthesizing cDNA from the RNA contained in said sample;
f) in vitro transcribing cRNA from said cDNA and labeling said cRNA;
g) hybridizing said cRNA to a gene chip followed by washing, staining, and scanning
h) acquiring a gene expression profile from said gene chip and analyzing the gene expression profile represented by the RNA in said sample; and
i) determining the gene expression profile in said subject of the minimal set of genes that classify the patient phenotype as healthy, febrile, or convalescent determined by the method described herein above;
j) classifying the subject in need thereof as being healthy, febrile, or convalescent by comparing the gene expression profile obtained in (i) to that of the classification assignment of healthy, febrile, or convalescent based on gene expression profile of the minimal set of genes as determined by the method described herein above.

The results procured by the present inventors provides a range of gene sets from a few genes to very large number of genes in various sets that could give the same percent correct classification results. The larger set size may provide a more robust prediction when the population involves more phenotypes. While the advantages and/or utility of the small set size may lie in the ability to make a quick independent diagnostic.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following figures in conjunction with the detailed description below.

FIG. 1 shows a diagram relating the two conditions used to handle blood collected in PAX tube. Condition E describes the isolation of total RNA from PAX tube collected blood after the minimum incubation time of 2 hours at room temperature, whereas condition O allows for an extended incubation time of 9 hours at room temperature followed by freezing at −20° C. for 6 days before RNA isolation.

FIG. 2 shows DNA contamination and removal. (A) DNA contamination of total RNA isolated from PAX tube even after on-column DNase treatment. Gel electrophoresis of real-time-PCR reactions for detection of gapdh DNA. Lane 1: molecular weight (MW) markers; lanes 2-7: gapdh 290 bp product amplified from total RNA isolated from PAX tube with on-column DNase treatment; lane 8: no template negative control. (B) In-solution DNase treatment removed contaminating DNA to a level undetectable by PCR. Gel electrophoresis of real-time-PCR reactions detecting gapdh DNA in various samples. Lane 1: MW markers; lanes 2 & 4: in-solution DNase treated RNA isolated from PAX tube; lanes 3 & 5: treated as in lanes 2 & 4, but without DNase; lane 6: cDNA positive control; lane 7: on-column DNase treated sample as positive control; lane 8: no template negative control. (C) RNA integrity was maintained after in-solution DNase treatment as determined by real-time RT-PCR. Lane 1: MW markers; lanes 2-5: cDNA from RNA samples used in lanes 2-5 of panel (B); lane 6: no reverse transcriptase negative control of sample corresponding to lane 4 in panel (B); lane 7: no template negative control.

FIG. 3 shows total RNA were of similar quality pre- and post- DNase treatment and between conditions. Bioanalyzer traces of fluorescence versus migration time of various total RNA samples. (A) Total RNA isolated from blood in PAX tube before DNase treatment. Black traces are from samples of condition E; gray traces are from samples of condition O. First peak at ˜23 sec is the marker control. Second peak at ˜41 sec is 18S ribosomal RNA. Third peak at ˜47 sec is the 28S ribosomal RNA. Large humps after ˜50 sec indicated DNA contamination. (B) Total RNA after DNase treatment. Descriptions are as in (A). (C) Comparison of pre- and post- DNase treatment traces. Black traces, one for each condition, are pre-DNase, whereas gray traces, also one for each condition, are post-DNase.

FIG. 4 shows characteristic profiles of double stranded cDNA, cRNA, and fragmented cRNA. Bioanalyzer traces of fluorescence versus migration time of various samples. Thick-dark-gray trace is a sample from condition E. Thin-black trace is a sample from condition O. Thick-light-gray trace is a no sample negative control trace. (A) Purified double stranded DNA. (B) Purified cRNA. (C) Fragmented cRNA.

FIG. 5 shows individual line charts relating the quality control metrics of various samples for HG-U133A and HG-U133B chips. Order of chips on the x-axis is based on the time of generation of the CEL file. UCL stands for upper control limit; LCL stands for lower control limit. The limits are set at ±3 standard deviations.

FIG. 6 shows gene-expression levels from the two conditions are highly correlated compared to related samples. Clustering dendrograms for HG-U133A (left panel) and HG-U133B (right panel) chips. The sample names with letters ‘E’ and ‘O’ correspond to samples processed at the same time as described in FIG. 1; also, sample names with the same letters designate technical replicates. Further descriptions for all samples are shown below the sample names. Each character encodes a sample descriptive ontology. For the Condition variable, ‘E’ designates samples processed similar to condition E, while ‘O’ designates samples processed similar to condition O. For Operator, ‘0’ designates one individual operator, while ‘1’ designates another operator. For Type of RNA, ‘T’ designates total RNA; ‘H’ designates IP RP HPLC purified mRNA; and ‘p’ designates polyA RNA. For Donor ID, each number represents a different volunteer.

FIG. 7 shows optimization of class prediction for non-febriles vs. febriles (A & B), healthy vs. convalescents (C & D), and febriles with adenovirus versus febriles without adenovirus infection (E & F). A, C, & E shows increments of the univariate significance alpha level (x-axes of A, C, & E), resulting percent correct classification (left y-axes) for various algorithms (color traces), and the number of genes in the classifier (right y-axes, black trace with filled circles); arrows indicate largest alpha level that resulted in the highest percent correct classification. In B, D, & F, at the optimal alpha level for each of the three classifications, classifier genes were further filtered by fold change level (x-axes of B, D, & F), with resulting percent correct classification (left y-axes) for various algorithms (color traces), and the number of genes in the classifier (right y-axes, black trace with filled circles); arrows indicate fold change level that resulted in the highest percent correct classification.

FIG. 8 shows cRNA profiles derived from Jurkat, Jurkat+Globin (JG), and paxgene RNA in different technical conditions. FIG. 8A—Elecropherograms for cRNA derived from JG RNA treated with biotinylated globin oligos (JGA), with PNA (JGP), no treatment (JGC) and Jurkat RNA with no treatment (JC). FIG. 8B—Gel view of cRNA derived from four RNA and showed the size of globin molecules (arrow indicated ˜0.8 kb) in JGP and JGC. FIG. 8C—Electropherograms for cRNA derived from paxgene RNA treated with biotinylated globin oligos (BA), with PNA oligos (BP) and no treatment (BC). FIG. 8D—Gel view of cRNA derived from BA, BP and BC RNA indicated the size of globin (arrow).

FIG. 9 shows Venn Diagrams demonstrating present call concordance among globin reduced Jukat+Globin RNA samples relative to Jurkat RNA and relationship among paxgene RNA in three different technical conditions. FIG. 9A—Identification of a control gene set (JCAP) commonly present in JA, JP and JC. FIG. 9B—There were additional 1394 genes present in JGA and JCAP relative to genes present in JGP and JCAP. FIG. 9C—Paxgene RNA followed by biotinylated globin oligos treatment resulting in additional 4159 (2607+1552) genes relative to no treatment of globin reduction (BC). At least 62.5% (2607/4159) were likely to be called present due to globin removal.

FIG. 10 shows Signal variation for each technical condition. FIG. 10A—Coefficient of variance (CV) vs. scaling signal intensities graph using all probe set data derived from Jurkat (J) and Jurkat+Globin (JG) RNA samples treated with biotinylated globin oilgos (JA, JGA), with PNA (JP and JGP) and no treatment of globin reduction (JC, JGC) were shown. FIG. 10B—CV vs. scaling signal intensities graph using all probe set data derived from paxgene RNA treated with biotinylated globin oligos (BA), with PNA (BP) and no treatment (BC). All of data were smoothed by Loess fitting with 2 degree freedom.

FIG. 11 shows multidimensional scaling cluster analyses performed on gene expression obtained from Jurkat RNA (J) and Jurkat RNA spiked in globin (JG) and paxgene RNA. All of probe sets with log raw signal intensity were used. FIG. 11A—Greater correlation within each triplicate resulted in a tight cluster for each triplicates. The triplicate clusters derived from Jurkat RNA with each technical condition were more closely located relative to any JG RNA. However, removal of globin (JGA, JGP) brought the triplicate clusters closer to Jurkat RNA relative to JGC. FIG. 11B—Triplicate for each paxgene RNA with different technical conditions was clustered more closely. Three technical variations resulted in three separate triplicate clusters.

FIG. 12 shows hierarchal cluster analyses performed on gene expression profiles for Jurkat and JG RNA and paxgene RNA samples. All probe sets on GeneChip Human Genome U133 plus 2.0 (approximately 56,000) with scaling signal intensities were shown on overview of gene expression profiles. The differentially gene expression profiles were obtained from Univariate test in Random Variance Model with false discovery ratio of 0.001. FIG. 12A—Overview of gene expression profiles among 18 samples representing Jurkat and JG RNA with three technical conditions. Globin removal from JG RNA by biotinylated globin oligos resulted in higher signal correlation to Jurkat RNA, thus, JGA triplicate and Jurkat RNA were clustered into the same group. FIG. 12B—Cluster analyses conducted by using differentially expressed gene profile among these 18 samples. The analyses resulted in 8614 differentially expressed genes and genes were divided into I, II, III, and IV based on JGA expression pattern. FIG. 12C—Cluster analyses performed on overall gene expression profiles derived from paxgene RNA. Globin removal from paxgene RNA by biotinylated globin oligos (BA) and PNA oligos (BP) exhibited more similar expression pattern relative to no globin reduction (BC). FIG. 12D—Class comparison analyses among 9 paxgene RNA samples resulted in 1988 differentially expressed genes.

FIG. 13 shows quality RNA derived from the PAX system of samples from the BMT population. (a) Overlay of electropherograms from BMTs with various phenotypes and handling conditions. The 18S and 28S ribosomal peaks are indicated. (b) Box plots of quality metrics calculated from the electropherograms. (c) Correlation between gapdh 3′/5′ values on the A arrays versus degradation factor (r=0.3, P=0.008, ANOVA). (d) Lack of RNA degradation over days elapsed from blood collection to processing. Samples marked by ‘+’, ‘x’, or ‘z’ had an additional thawed-froze cycle before final thawed for RNA isolation. (e) Correlation between the Mean Corpuscular Hemoglobin (MCH) and number of probesets called Present in the B arrays, (r=−0.272; P=0.008, ANOVA). Line shown is from equation: Number Present=8108−117 MCH

FIG. 14 shows gene expression profiles of the BMTs. To remove undetected transcripts, those with >80% absent calls across samples were filtered resulting in 15,721 from 44,928 probesets. To remove uninformative transcripts, probesets in which less than 20% had a 1.5 fold or greater change from the probeset's median value were removed, resulting in 7682 probesets. To focus on transcripts with differences in expression among the four infection status phenotypes, those probesets with P>0.01 by ANOVA were excluded, resulting in 4414 probesets. The heat-map shows the transcript abundance (green to red intensities) detected by these 4414 probesets (rows) in each blood sample (column). The rows were hierarchically clustered with 1-correlation distance and average linkage, while the columns were sorted into the infection status phenotypes. Top blue, brown, yellow, and light blue bars denote samples from healthy, febrile without and with adenovirus, and convalescent patients, respectively. Bottom scale denotes standardized values for the green to red intensities in the heat-map. Side gray, orange, and purple bars denote clusters of transcripts that differ among the phenotypes.

FIG. 15 shows optimization of class prediction for non-febrile vs. febrile (a), healthy vs. convalescent (b), and febrile without adenovirus versus febrile with adenovirus infection (c) phenotypes. Shown in the lower left corners of the three panels are the estimated optimal P-value cut-off levels for each of the three classifications. Classifier transcripts were further filtered by fold change level (x-axes), with resulting percent correct classification (left y-axes) for various algorithms (color traces), and the number of probesets in the classifier (right y-axes, beaded black trace); arrows indicate fold change level that resulted in a highest percent correct classification.

FIG. 16 shows identities and expression of genes in classifiers found from class prediction analysis. In each panel, top bar indicates the classification phenotypes of the samples (columns). Panel a has a second bar that further indicates healthy, convalescent, febrile without and with adenovirus samples as blue, light blue, brown, and yellow, respectively. The middle set of color bars in each panel mark samples that were misclassified (black) by various algorithms. The heat-maps indicate relative expression levels of genes (green to red intensities) identified by gene symbols on the right; for cDNA clones without gene symbols, probeset identifiers are displayed instead. Dendrograms are from clustering of standardized transcript levels (rows) using 1-correlation distance and average linkage. Bottom scale denotes standardized values for the green to red intensities in the heat-map. The transcript sets in panels a, b, and c gave results marked by arrows in FIGS. 3a, b, and c, respectively.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention provides a method for identifying human gene transcripts in blood, and their expression patterns, to identify a causative agent of respiratory infection, and provide a measure of recovery during the period of time following infection. The methods developed here can be extended to the discovery of gene expression profiles that will be indicative of exposure and predictive for the actual development of disease. These abilities have not previously been demonstrated in a human population.

Gene Expression:

The following description details the importance of the present invention and its utility in gene expression analysis:

1. Identification of uncultivatable organisms: Mycoplasma pneumoniae, Bordetella pertussis and Chlamydia pneumoniae, which commonly cause respiratory disease in all age groups. These organisms require special transport media for sample collection of respiratory secretions. Even with optimal transport, it is tremendously difficult to cultivate these common organisms; therefore, healthcare workers are often unable to make a diagnosis and have little opportunity to direct antimicrobial therapy to potentially shorten the duration or to prevent transmission of disease with these organisms. Bordetella pertussis is the causative organism for whooping cough in children and carries a high morbidity. Adults infected with this organism often develop prolonged, dry cough and remain undiagnosed during the period of infectivity and possible transmission. It is likely that adults represent a typically undiagnosed reservoir of disease for this organism that can have significant impact on the health of children.

2. Analysis of organisms for which no sample can be taken, for example TB from children. Young children tend to have disseminated tuberculosis infection and will not tend to have a productive cough; this means that it is very difficult to collect sputum to look for the organism. Having an assay in blood that detects an immunologic signature for tuberculosis infection and disease in children would be a significant medical breakthrough. Worldwide, tuberculosis is a significant cause of morbidity and mortality in children, especially in impoverished regions of the world. Early detection of infection can significantly limit disease. Therefore, this area is of particular interest in the present invention.

3. Analysis of and identification of multiple organisms in a single blood sample.

4. Differentiation of a pathogen from colonization (discussed further below).

5. Determination of pre-symptomatic-exposed individuals.

6. Expansion to non-infectious/toxin exposure.

7. Identification of normal baseline for comparison for all studies.

Based on the foregoing and the embodiments specifically described herein, the present invention provides an opportunity to direct treatment options. In other words, by determining the gene expression patterns (both baseline healthy and ill) the artisan would be enabled to determine the diagnosis and the corresponding treatment, i.e. whether an individual has a bacterial infection—give antibiotics or viral infection—no antibiotics. In this manner the medical professional may reduce inappropriate antibiotic use and decrease resistance.

Further, the present invention may be employed to measure response to treatment—i.e., is there evidence that the host is resolving the infection? At times, individuals will be hospitalized and treated for respiratory infection, they appear to get better, but then develop fever again—the causes of fever can be: new infection—intravenous line is now infected or patient has developed urinary tract infection due to indwelling Foley catheter—typically multiple tests have to be sent—blood, urine, sputum to determine whether there is a new site of infection. Also, diseases like pancreatitis or cholecystitis that develops in very ill patients while hospitalized can be non-infectious causes of fever that develops after admission. Gene expression as described herein provides a means to take a single sample, blood, and differentiate infectious from non-infectious cause of fever and to identify whether a new pathogen at a new anatomic site is responsible for the new fever—e.g., if an individual was admitted with S. pneumoniae pneumonia and had gene expression pattern consistent with this, but then developed a new fever in the hospital and had a changing gene expression pattern consistent with a S. aureus (skin pathogen) infection, then the new gene expression pattern would direct the practitioner to look at IV sites and other skin sites, such as decubitus ulcers, for a new source of infection. If the gene expression pattern did not appear to be consistent with a response to an infectious agent, then the practitioner should consider diagnoses such as pancreatitis or cholecystitis. The development of fever during hospitalization is not uncommon and often is a vexing problem for the health care practitioner, especially in severely ill patients in the Intensive Care Unit. Therefore, techniques as described herein would be well received in the medical profession.

The present invention was accomplished following successful adaptation of a commercial technology (Affymetrix Human Genome U133 chip set) that has not been demonstrated prior to this to be effective for whole blood expression profiling due to interferences from high-abundance globin RNA (20). The demonstration of the enablement of the present invention has been assisted, in part, by the employment of enhanced sample preparation methods (e.g., PAXgene™). Further, by employing rigorous screening and control functions the present invention offers a significant advantage in that the data obtained thereby are free from the confounding environmental influences that pervade other gene monitoring studies. Moreover, the gene products used to distinguish between varying febrile respiratory disease states can be targeted for a variety of other assay types that do not require whole genome transcriptional monitoring or the attendant processing steps.

Herein, the present inventors demonstrate that high density DNA microarray technology can be adapted for insertion into an accelerated system for discovery of blood transcriptional markers of infectious disease and other factors important of health, occupational, and military significance.

When considering host gene expression profiling, the capacity to conduct thousands of assays simultaneously poses challenges regarding data analysis, storage, and management. While data storage and management issues are largely technical concerns for information technology specialists, no clear consensus on analysis techniques has emerged for making use of host gene expression profiles. The major role for bioinformatics is the identification of patterns associated responses to pathogens which may not only provide a means of detection, but also elucidation of genetic networks underlying initiation and progression of disease. The most commonly exploited tool for analysis of gene expression profiles is hierarchical clustering (21, 22) where the fundamental assumption is that similar trends, computed through a measure of distance, in the relative magnitudes of gene modulation imply similarity of function.

A critical need for the interpretation of large data files is the visualization of information, which can be readily accomplished by dendrograms that can be derived from cluster analysis. Interpretation of expression profiling data has been used to gain profound insights into gene function. Clustering of genes expressed in yeast coupled with statistical algorithms yielded a model of regulatory transcriptional sub-network (23). A significant demonstration of the utility of clustering has been offered by Hughes et al. (24), where a compendium of expression profiles of 300 diverse yeast mutations was used to identify novel open reading frames that encoded proteins of several cell functions. In regard to pathogen detection, different pathological conditions reflected by particular expression profiles could also be clustered (clustering by arrays rather than by genes), but variation among a broad set of genes or dimensions may reduce the ability to discern pathogen exposure states.

Efforts in functional genomics related to cancer research have yielded major successes in the pursuit of gene expression signatures. Expression-based criteria or class predictors have been defined based on neighborhood analysis (25), Bayesian regression models (26), and artificial neural networks (27-29). These predictors were successfully used to classify novel samples in a manner consistent with clinical assessments. In fact, classifications based on gene expression alone or class discovery has also been demonstrated, suggesting that gene expression profiling has the capacity to identify subtypes that have not been previously defined (25).

While promising, one should note that cancer line gene expression analyses are one-dimensional; in contrast, a host expression profile evoked by pathogen exposure would be expected to be temporal and “dose-dependent”. Comprehensive sets of gene expression profiles that explore temporal and dose ranges for pathogen exposure must be produced to map the continuum of gene expression changes.

The present invention has been developed, in part, based on the rigorous assessment of the RNA quality from PAX tubes from a relatively large sample of humans with various disease phenotypes, to determine the following: nested sets of genes that could optimally classify the four phenotypes of (a) healthy, (b) recovered, (c) febrile with adenovirus infection, and (d) febrile without adenovirus infection; lists of differential genes among the four phenotypes; and the pathways in blood cells involved in respiratory disease due to adenovirus infection versus non-adenovirus infection. These results demonstrate possibilities and issues involved in measurement of gene expression from whole blood at the population level; show the potential of using host gene-expression responses in blood cells to distinguish pathogen classes; elucidate functional pathways involved in adenoviral respiratory disease; and provide a data set to develop statistical models to answer other biological questions of interest.

The present invention was accomplished as a result of the availability of the BMT population of the U.S. Air Force to the present inventors. The BMT population offered advantages for surveillance studies. The major advantage is that the BMT population is racially and ethnically diverse and is representative of the racial/ethnic diversity observed in the United States. The BMT population undergoes environmental factors similar to those of other populations to include: smoking, exercise, stress, schooling (education), activities of daily living; while the activities of daily living may appear to be more regimented than their civilian counterparts, they largely reflect typical schedules (early breakfast, exercise, education for 6 hours, regular lunch and dinner, cleaning of dorms or TV in evening). These characteristics are advantageous for many research questions. One difference between the BMT and the civilian population is that there is a predominance of males in the BMT population (90% male, 10% female) and the age range is typically from 18-25 years. In order to address this, the present inventors are extending this study to a civilian population that includes individuals of all ages greater than 18, male and female, who present to medical clinics and hospital wards with symptoms of upper respiratory tract infection. The ability to ascribe differential gene expression profiles in a relatively homogeneous population is directly applicable to military applications and is enabling for the development of methods necessary for the discovery of a subset of markers that will be predictive for a larger population.

Sample Preparation

There has been considerable speculation within the research community that blood would provide the best range of gene expression biomarkers involved with the immune response to a broad range of viral and bacterial infections. A variety of blood cell isolation kits and reagents might be useful for collecting blood cells and isolating RNA for gene expression analysis, including CPT vacutainer tubes (Beckman Dickenson) which collect blood and after a spin can segregate the PBMCs; the Paxgene blood RNA system, which has an RNA stabilizer reagent inside the vacutainer tube for blood collection; and the Tempus blood collection tube from Applied Bioscience which also has a stabilizer, but is relatively new on the market.

Relman (18) has used PAXgene to successfully measure gene expression changes in blood using cDNA and long oligonucleotide (70-mer) microarrays. However, the stability of RNA in PAX tubes over handling conditions practical for multicenter surveillance was not assessed. Relman (18) processed all the PAX tubes within 24 hours of collection, which is not practical for large multicenter surveillance. Also, in principle, a higher degree of sequence resolution would be obtainable using shorter (25-mer) oligonucleotide arrays have high-density probe tiling (e.g. Affymetrix GeneChip) that blanket entire genomic regions of interest. However, prior observations have been that PAXgene produced an insufficient number of “percent present” calls (i.e. the percentage of total genes determined to be measurably expressed as determined by the Affymetrix GCOS gene expression software) on Affymetrix GeneChip expression microarrays. Presumably, the unsatisfactory level of “percent present” calls was caused by the interference of high abundance globin RNA on binding of lower abundance transcriptional markers. Thus, there have been no prior reports of the combined use of PAXgene blood RNA kits and the Affymetrix GeneChip® platform prior to that described herein.

From a logistical perspective, the use of PAXgene technology would be highly preferred for discovery of expression markers during opportunistic encounters of infectious agents with a mobile human population. This is because of the proposition of the unique abilities of the PAXgene reagents to rapidly terminate gene expression in cells and stabilize RNA at the time of blood draw, minimizing the confounding effects of variable RNA degradation and gene expression perturbations caused by varying storage and processing times and conditions in a military clinical setting, rather than controlled laboratory environment using controlled exposures and sampling times. Traditionally, studies of blood cells utilize gradient-density based methods to collect live mononuclear cells for analysis such as cell sorting, genotyping, and expression profiling. However, the RNA population may have changed or become degraded due to the processing of live cells, as transcript levels can fluctuate early after blood collection (30-32). Additionally, these methods do not isolate neutrophils, which typically pass through the gradient-density and are not collected for analysis. These methods are labor intensive and do not translate well to mobile populations. In contrast, the PAX tube contains a proprietary solution that reduces RNA degradation and gene induction as 2.5 ml of blood is flowed into the tube (30-32). However, the blood cells are killed and cannot be sorted, nor can DNA be isolated using procedures described in the PAX kit handbook (33).

Since the goal of the present inventors is to measure RNA transcript levels for diagnosis or epidemiologic surveillance, we decided that the RNA stabilization capability of the PAX tube complemented our interests, especially for situations where one cannot process the blood samples soon after collection. It is to be understood that alternative sample preparation methods may be used in the methods of the present application, so long as these alternative sample preparation methods do not compromise the integrity of the RNA material contained within the sample.

In view of the foregoing, the present inventors have developed a modified protocol for gene-expression analysis of RNA isolated from human blood collected and processed with the PAXgene Blood RNA System that works with the Affymetrix GeneChip® platform. The protocol was used to compare profiles of blood samples collected in PAX tubes that were handled in two ways that may provide practicality to surveillance and clinical studies (conditions E and O). These methods entailed collecting blood samples in a PAX tube and then either, (a) incubating the sample for a minimum of 2 hours at room temperature (condition E) and then isolating RNA from the PAX tube-collected blood samples, or (b) incubating the sample at room temperature for nine hours followed by storage at −20° C. for 6 days (condition O) and then isolating RNA from the PAX tube-collected blood samples.

The present inventors found differences between the two handling methods (although either of these conditions may be employed in the context of the present invention). Samples of condition E had higher DNA contamination, lower total RNA yield, and higher double-stranded cDNA yield than samples of condition O. ANOVA indicated that the two conditions contributed to differences in gene expression levels, but the magnitude was minimal, being 0.09% of the total variation. These results should facilitate incorporation of expression profiling protocols and handling methods into clinical and surveillance level procedures.

Genome-wide expression studies of human blood samples in the context of clinical diagnosis and epidemiologic surveillance face numerous challenges—one of the foremost being the capability to produce reliable detection of transcript levels. Many factors contribute to the variability of target detection, including: the method of blood collection, sample handling, RNA stabilization, RNA isolation, and other downstream processes.

The Affymetrix® GeneChip® platform can measure a significant subset of the transcriptome. In design, it incorporates a DNA oligonucleotide microarray, manufactured via photolithography to detect labeled cRNA targets amplified from RNA populations. However, some labs have observed a lower percentage of genes detected using RNA from whole blood compared to RNA from mononuclear cells regardless of the blood collection or processing method. This phenomenon may be due to the dilution of leukocyte RNA by RNA from reticulocytes, the activation of leukocytes during the isolation procedure, and/or the degradation of RNA isolated from the PAX tubes.

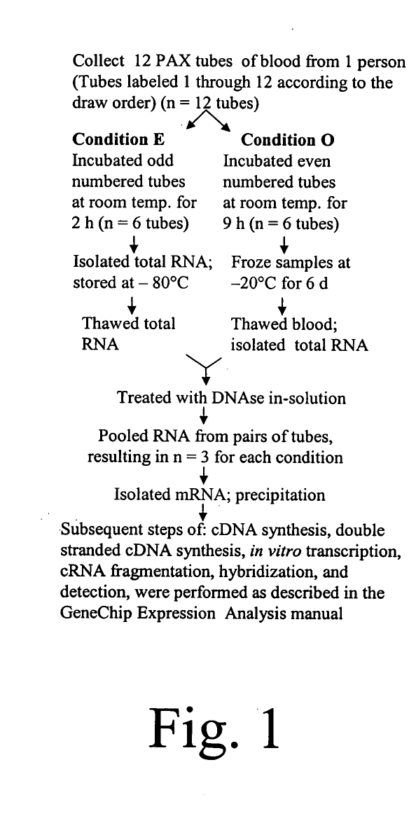

The RNA, isolated from blood in PAX tubes that is stored at room temperature, at −20° C., at −80° C., or after freeze-thaw cycles has been shown to be stable as determined by ribosomal RNA bands on agarose gel, fluorescence profiles on the bioanalyzer (Agilent Technologies), or RT-PCR for a few genes (31, 34-45). However, the integrity of the RNA at the transcriptome level as measured by Affymetrix microarrays has not been determined. In the context of multi-centered epidemiological studies, one needs to stabilize the transcriptome at the point of sample collection and during sample storage and transportation. Therefore, we compared the gene-expression profiles of parallel blood samples drawn into PAX tubes handled in two ways (Condition O and E described above) (FIG. 1). In the first way (FIG. 1, Condition E, as in fresh), RNA was extracted after the minimum incubation time of 2 hours from phlebotomy; while in the second way (FIG. 1. Condition O, as in frozen), the blood sat for 9 hours at room temperature followed by storage at −20° C. for 6 days, followed by RNA extraction. If there were no differences between these two methods as related to gene expression, then this would allow for a reasonable time frame before the samples have to be processed or frozen for transportation or later processing. Otherwise, one needs to consider the magnitude of the differences and weigh its contribution to transcriptome variability versus the flexibility, practicality, and feasibility of sample handling, storage, and processing.

In the present specification, the present inventors relate a quality assured and controlled protocol that is capable of producing reliable gene-expression profiles, using the GeneChip® system and RNA isolated from whole blood using the PAXgene™ Blood RNA System. We used this protocol to compare quality control (QC) metrics and gene-expression profiles of PAX tube collected blood that was handled by the methods diagramed in FIG. 1. These results direct protocols for clinical studies and progress us towards the goal of using the transcriptome in diagnosis and surveillance.

Our results implied several recommendations as to sample handling for multi-centered studies. Since there were differences between the conditions but they both showed good within-group reliability, one should preferably pick one method to reduce variability. In which case, condition O seemed advantageous over E, as it provided time before one had to process or freeze the samples and allowed for transportation while frozen. If one needed the flexibility of the range of handling methods between the conditions, then this would still be possible, as long as during subsequent analysis, one increased statistical stringency.

Therefore, in a preferred embodiment of the present invention blood samples are obtained and prepared for microarray analysis by the following general protocol:
(a) Blood Collection
   Preferably using PAX vacutainer tubes which has RNA stabilization reagent;
   Alternatively, the skilled artisan may use capillary tubes to obtain a few drops of blood then place in RNAstat to stabilize RNA;
   Another alternative is the use of Tempus tubes from Applied Biosystems, which also have RNA stabilizing reagent;
   Also within the scope of the present invention, the skilled artisan may use single cells from drops of blood and pass the sample through microfluidic channels to different stations that measure different things about the cell including the transcriptome. In so doing, this technique may provide sufficient rapid measurements that one does not need to stabilize RNA;
(b) Target RNA Isolation
   Preferably using PAX tubes, the PAX kit system is used to isolate target RNA with modifications to the manufacturer's instructions (described herein elsewhere);
   Other kits that are commercial available and may be used in the present invention include those available from Qiagen (e.g., Qiamp), or from Zymogen, or from Gentra to isolate RNA from whole blood not in stabilizing solution;
   Also suitable for use are robotics system available for purifying RNA from blood in a high-throughput manner;
(c) Labeling and/or Amplification of Target RNA
   Preferably, for amplification of the target RNA, the purified RNA is reversed transcribed to cDNA then to double stranded cDNA with a T7 promoter for subsequent in vitro transcription to amplify and label the resulting cRNA target;
   Alternatively, if enough RNA is isolated from blood, then one could label the RNA directly with fluorescent dye or other molecules of high light output for high sensitivity of detection, thus providing a time savings;
   Other RNA amplification and strategies may also be employed, including, but not limited to, the Ovation RNA amplification technology (Affymetrix) using one-cycle and two-cycle to reduce initial amount of RNA needed and also to reduce processing time;
(d) Hybridization onto microarray
   Preferably, using the Affymetrix hybridization oven for 15 to 17 hours at 45° C. of hybridization of labeled target onto the Genechip microarray. Conditions, including incubation time and temperature, may be further modified, so long as sensitivity and accuracy are maintained.
   Other platforms (described elsewhere) may be suitable for use in the present invention in which one may be able to reduce the hybridization time;
(e) Detection of Bound Target RNA
   Preferably, using strepavidin phycoerythrin to bind the biotin on the target RNA, followed by further signal amplification with biotinylated anti-strepavidin antibody and another staining with strepavidin phycoerythrin to increase sensitivity;
   Alternatively, one can replace this step with a molecule that can emit more light without much quenching. Examples of such molecules include: quantum dots, alexi dyes, orbiotinylated viruses. Thereby, detection and/or hybridization times may be shortened;
(f) data integration and analysis.

Although the PaxGene-based methods worked well in the present invention, the present invention contemplates and includes additional optimized processes. One adjustment to the existing protocol is to omit the increase in proteinase K during RNA isolation. To this end, some reports have stated that sufficient pellet formation is possible by simply increasing centrifugation time. Therefore, it is al so possible to increase the centrifugation time concomitant with the omission of the proteinase K increase. Alternatively, the protein K digestion step may be shortened by using a more concentrated proteinase K and a shorter incubation time. Also, the eluent volume during mRNA elution was 100 μl, but a 200 μl total eluent might give better yield. The in-solution DNase treatment was used to ascertain removal of DNA. However, the amount of DNA left after on-column DNase treatment might not interfere with subsequent steps.

Further, to improve preparation time on the PaxGene technology itself, vacuum-filtering methods may be employed to collect the cells rather than spinning the tubes to pellet the cells. Another permissible modification would be to use filtering methods to collect the supernatant after proteinase K digestion rather than spinning down the debris for a defined time (e.g., 30 min). Robotic systems could also be employed to considerably shorten liquid handling time.

For alternatives to existing protocols, other related sample collection methods and transcriptome measurement technologies may be used. These include:
1) The Tempus™ Blood Collection Tube from Applied Biosystems;
2) The CPT™ Cell Preparation Tube from Becton Dickenson, which can collect live cells and isolate peripheral blood monocytes after a spin down;
3) Nanoarrays of oligomer probes on nano wires and transcriptome measurements from single cells flowing through microfluidics channels;
4) Microcapillary tubes to collect a few drops of blood perhaps followed by lysing of the red blood cells and storage in RNALater for RNA stabilization. Then, when needed, the RNA can be extracted from blood cells using other kits such as the Qiamp kit from Qiagen or the blood RNA isolation kit from Zymogen.

Additional alternative and/or supplemental preparation methods are also contemplated, which may shorten duration time and reduce initial input RNA amount, for Example:
1) The new method published by Affymetrix that can label total or polyA RNA directly without amplification (46) (Cole K, et al. “Direct labeling of RNA with multiple biotins allows sensitive expression profiling of acute leukemia class predictor genes.” Nucleic Acids Res. 2004 Jun 17;32(11):e86.);
2) Direct chemical labeling of the RNA, for example by the method of Label IT® μArray™ Biotin Labeling Kit by Mirus;
3) The Ovation kit available from NuGEN Technologies, Inc., which can generate a large quantity of RNA using only 15 ng of RNA in 4 hr. This technology might even allow direct substitution of the PAX system, as only a few drops of blood would be needed;
4) The Dynabeads® mRNA DIRECTTM Kit from Dynalbiotech, which uses magnetic beads to extract mRNA in 15 min in a single tube. Can be performed using whole blood.
5) The MessageAmp™ II aRNA Amplification Kit available from Ambion.

Other methods that are also contemplated to increase sensitivity of the sample preparation processes include:
1) Adding unlabeled globin RNA or DNA to the hybridization step to block background, thereby perhaps increasing detection calls;
2) Removal of the globin mRNA via magnetic beads isolation; and
3) Adding more cRNA onto the chips and/or background reduction as in item #2.

As stated above, the present invention was accomplished following successful adaptation of a commercial technology (Affymetrix Human Genome U133 chip set) that has not been demonstrated prior to this to be effective for whole blood expression profiling due to interferences from high-abundance globin RNA (20). Therefore, globin reduction for whole blood RNA is an important step for improving gene expression profile from whole blood sample, since 70% total RNA in whole blood samples are globin mRNA, which would result in decreased percent present calls, decreased call concordance and increased signal variation.

In Example 4, the present inventors evaluated biotinylated globin oligos (Ambion) and PNA oligos (Affymetrix), which prove to be the two most effective methods to reduce globin mRNA from whole blood RNA. However, heretofore there was no systematic comparison on gene expression profiles derived from these two methods. The present inventors' studies using Jurkat RNA and globin spiked in Jurkat RNA (JG) in parallel with paxgene RNA provides a detailed insight of comparison between these two methods for cRNA profiles, present calls, call concordance, signal variation, multidimensional scaling and hierarchal cluster analysis in gene expression profiles.

Although neither of two globin reduction methods gave the same gene expression profile (gxp) as Jurkat RNA, the globinclear method using Biotinylated globin oligos gave closer gxp than PNA method. The data set forth in Example 4 indicate that the globinclear RNA resulted in significantly higher number of present calls (%), higher call concordance %, lower false negative discovery, and closer gene expression profile to no globin control relative to the single step PNA reduction method in Jurkat and JG RNA. However, it also resulted in higher signal variation, lower triplicate correlation coefficient and no difference in correlation to no globin control relative to the PNA method, possibly due to the multi-step procedure that involves a 2 hour processing time. It is notable that highly pure RNA free from RNase contamination is required for the globinclear method, necessitating in solution Dnase digested paxgene RNA to be subjected to cleaning and concentration using the Rneasy Minelute column (Qiagen). In contrast, the single step PNA process is easy to perform simply by adding the oligo mixture to the downstream application tube. But we noticed that higher ratios of 3′/5′ GAPDH and 3′/5′ Actin appeared in paxgene RNA samples and smaller cRNA size in PNA treated paxgene RNA. Reduction in cRNA size may lead to a higher ratio of the two control probe sets and likely is the cause of the higher CV seen with paxgene RNA.

PNA oligos specifically hybridized to the 3′ end of globin mRNA to prevent reverse transcription, while biotinylated capture globin specific oligos hybridized to globin mRNA followed by removal of globin mRNA via strepavidin magnetic beads. Thus, because the globin clear method physically separates globin mRNA from the sample, it allowed non 3′ bias techniques downstream, such as direct labeling of globinclear RNA for target preparation. Globinclear method produces a good quality RNA with the ratio of 260/280 beyond 2.0. However, from paxgene RNA not from J and JG RNA, the cRNA yield reduces to half of the amount of no treatment or PNA treated sample and at least 5 μg paxgene RNA is required to get enough cRNA for hybridization. Whereas, 1 μg paxgene RNA treated with PNA oligo is able to amplify enough cRNA (approximately 20 μg) for hybridization

In sum, the present inventors have compared pros and cons for the globinclear and PNA methods. Based on this comparison, the present inventors have found that the both of these methods may be used to reduce the amount of globin in whole blood RNA. Choice of methods depends on the individual project setup and goals. However, in either scenario by employing one of these methods a significantly higher number of present calls (%), higher call concordance %, lower false negative discovery, and closer gene expression profile to no globin control can be obtained.

Based on the foregoing, the present inventors have developed a method for identifying gene expression markers for distinguishing between healthy, febrile, or convalescence in subjects that have been exposed to one or more of various infectious pathogens.

In general, a preferred method of the present invention is as follows:
a) sample collection;
b) Isolation of RNA from said sample;
c) Removal of DNA contaminants from said sample;
d) Optional concentration and clean-up of RNA;
e) Spike-in controls for normalization;
f) Optional globin mRNA reduction/elimination;
g) Synthesis of cDNA;
h) IVT (in vitro transcription) labeling and cRNA synthesis;
i) cRNA quantification and quality control;
j) Gene chip hybridization, wash, stain, and scan;
k) Optional second gene chip hybridization, wash, stain, and scan;
l) Data acquisition and management; and
m) Statistical analysis.

Within the context of the present invention, including this preferred embodiment, the sample is preferably whole blood. However, within the context of the present invention, any RNA source may be utilized whether from whole blood or extracted from some other source. In a preferred embodiment, and as described above and in the Examples, when the sample is whole blood the collection device is a PAXgene blood RNA tube.

Within the context of the present invention, including this preferred embodiment, the RNA may be isolated by any known RNA isolation technique. As stated above, the RNA isolation technique may be facilitated by use of a commercially available kit, including the PAX kit system or Qiamp. Preferably, RNA isolation may be performed without on-comun Dnase treatment. In addition, in an embodiment of the present invention, RNA isolation may be performed with a Qiashredder column (Qiagen Corp.), which helps to increase the yield of RNA obtained from samples obtained from sick subjects.

Within the context of the present invention, including this preferred embodiment, the DNA may be removed by any known technique. In a preferred embodiment, the DNA is removed from the sample by in-solution Dnase treatment. The Dnase treatment may be performed with or without use of an inactivation reagent. In the case of use of an inactivation reagent, it is preferred that the inactivation reagent be added after a defined period after onset of Dnase treatment. In this case, the defined period is preferably set by the level of DNA remaining in the sample. In case where the DNase inactivation reagent is not used is because subsequent use of column to clean (hence DNase and metal ions are removed) and concentrate RNA for globinclear method.

Within the context of the present invention, including this preferred embodiment, the RNA may be concentrated and cleaned-up where necessary. For subsequent techniques in the preferred protocol of the present invention it is preferred that there be a total of at least 8 μg of RNA initially before going into column to clean and concentrate. As such, one or more of several techniques may be used to concentrate and clean-up the RNA. For example, a Minelute column may be used and the RNA eluted in BR5. Also it is possible to used ethanol precipitation techniques with resuspension in water although this is not compatible with globinclear downstream as this method does not clean the RNA enough (e.g., approximately 10 μl). Further, to determine whether additional concentration and/or clean-up is necessary the RNA and/or quality thereof may be assessed on a bioanalyzer or a nanodrop.

Within the context of the present invention, including this preferred embodiment, it is preferred for the subsequent steps (i.e., steps (e)-(m)) that the starting amount of total RNA be at least 5 μg, although 1 μg starting amount can work with PNA and no globin reduction methods.

Within the context of the present invention, including this preferred embodiment, it is important that prior to cDNA synthesis that a spike-in control be added to the reaction cocktail containing the subject RNA. This step is critical for normalization between diseases and patients and poses an improvement over existing techniques. The spike-in control for use in the present invention is preferably a polyA control or an ERCC universal control (http:H/www.cstl.nist.gov/biotech/workshops/ERCC2003/).

As stated above, 70% of mRNA in whole blood samples are globin mRNA, which would result in decreased percent present calls, decreased call concordance and increased signal variation. As such, in a particularly preferred embodiment, the globin RNA content is either reduced or eliminated. To this end, the term “reduced” is contemplated as meaning that there is a reduction in the total amount of globin RNA in the sample of at least 50%, preferably at least 60%, more preferably at least 70%, even more preferably at least 80%, still even more preferably at least 90%, and most preferably at least 95% as compared to the sample prior to the reduction treatment. Within the context of the present invention, the globin RNA reduction may be performed using biotinylated globin capture oligos (Ambion globinclear kit) or PNA (Affymetrix GeneChip globin reduction kit) according to modified manufacturers' procedures (see the Examples of the present invention).

When the globin RNA reduction method is that of using biotinylated globin capture oligos, it is preferred that biotinylated globin capture oligos are added to the total RNA and, subsequently, the globin mRNA were removed by contacting the RNA mixture with streptavidin beads (e.g., Strepavidin magnetic beads). Globinclear RNA was further purified using magnetic RNA bead. Alternatively, it is possible to replace the magnetic bead based total RNA isolation step with Qiagen column chromatography. In either event, the subject RNA is preferably eluted with water or BR5 (preferably diluted such that following speedvac concentration the total salt content is lx BR5 or if water is used for elution, then speedvac to small volume and then increase to appropriate volume using BR5). Accordingly, when the globin RNA reduction method is that of using biotinylated globin capture oligos is employed it is a highly preferred embodiment that the RNA be concentrated and cleaned-up before and/or after said method. It is important to note that the Elution buffer that comes with the Globin clear kit does not work with downstream speed vac concentration and affymetrix target prep. Ambion test their Elution buffer with their Message Amp target prep method, whereas the present invention preferably uses Affymetrix target prep.

When the PNA method is used as the RNA reduction method, this step is performed simultaneously with cDNA synthesis. In this method, PNA is spiked in with the cDNA synthesis cocktail. Peptide nucleic acid (PNA) oligonucleotides specifically bind to the 3′ end of globin mRNA to inhibit reverse transcription during cDNA synthesis. However, when employing this method, care must be taken to preserve the stability of PNA and one has to take measures to prevent PNA aggregation and precipitation. It may also be advisable to run Jurkat globin as a control for efficient globin removal.

When the method above is practiced in the absence of a globin RNA reduction protocol low sensitivity and high variance are observed. When the PNA method is followed the sensitivity is boosted, low variance is observed, but this method only works for 3′ biased reverse transcription assays. When the biotinylated globin capture oligo method is followed the best sensitivity is obtained, low variance is observed, and the RNA may be used for nay reverse transcription assay including non-3′ biased assays. With the biotinylated globin capture oligo method very high quality RNA is required, whereas the PNA method is useful even without high quality RNA. It is important to note that if ERCC controls are uses, then the data can be normalized across highly different gene expression profiles.

Within the context of the present invention, including this preferred embodiment, it is preferred that the purified target RNA be amplified via reverse transcription to cDNA utilizing a T7 polyT primer (or a random primer for non 3′-biased assay alternative for exon arrays) then to double stranded cDNA with a T7 promoter for subsequent in vitro transcription. Following production of double stranded cDNA, the double stranded cDNA should be cleaned-up and concentrated as appropriate.

Within the context of the present invention, including this preferred embodiment, commercially available in vitro transcription kits are preferably used to amplify and label the resulting cRNA. Examples of such kits are readily available through Enzo Biochem or Affymetrix. These methods may be performed as instructed by the manufacturer with a subsequent cRNA clean-up as appropriate.

Within the context of the present invention, including this preferred embodiment, the cRNA is quantiated and the quality of the sample assessed to determine the cRNA yield and purity of the sample, respectively. To determine whether additional concentration and/or whether further clean-up is necessary the RNA and/or quality thereof may be assessed on a bioanalyzer, nanodrop, and/or UV spectrophotometer (cuvette or plate reader). If necessary, if an increased cRNA yield is necessary, Ambions Message Amp kit may be used in accordance with the manufacturers' instructions. Among the quality controls within this embodiment are the ratio of 260/280, the yield of cRNA, etc.

Within the context of the present invention, including this preferred embodiment, gene chip (first, second, or subsequent chips) hybridization, washing, staining, and scanning may be conducted as directed by standard Affymetrix protocols. For example, hybridization may be conducted by contacting approximately 10 μg of biotin incorporated cRNA to the genechip in the Affymetrix hybridization oven for 15 to 17 hours at 45° C. of hybridization of labeled target onto the Genechip microarray. Conditions, including incubation time and temperature, may be further modified, so long as sensitivity and accuracy are maintained. In addition, the washing and staining conditions may also be modified so long as the sensitivity and accuracy of the technique are maintained. The nature, identity, and composition of the genechip for use in the present invention are not limited; however in a preferred embodiment the genechip is selected from Affymetrix U133A, U133B, and U133 plus 2.0. In a preferred embodiment, it is preferred that either U133 plus 2.0 or both U133A and U133B are used as the genechip.

As discussed below, data acquisition and handling may be performed by any means known by the skilled artisan. For example, data acquisition and handling may be performed by hand and passing through various programs, including the manufacturer developed software accompanying the genechip reader.

A more complete discussion of data management and statistical/functional analysis is provided in the description below and the Examples that follow.

However, briefly, data management is conducted by using Affymetrix GCOS gene expression software data are exported to Excel. MAS5.0 signal and present calls are exported and saved as tab-delimited text files, as are scaled and unscaled Signal values, to test normalization assumptions and strategies. The text files (and file names) are subsequently reformatted for import into Arraytools in house R-script. QC analysis software, datamatrix, and JMP IN (SAS Institute) programs are used for analysis of variance and further data exploitation. Where appropriate, the data for U133A and U133B are joined in Arraytools.

For analysis software the following can be mentioned:
Statistical analysis software: SAS and JMP;
Class Prediction analysis software: BRB-Arraytools;
Clustering analysis software: BRB-Arraytools and dChip; and
Functional analysis software: EASE, DAVID, Pathway Assist, and Iobion Stratagene.

To identify gene expression profiles resulting from pathogen exposure and to enable the general technology described herein, the following program was undertaken with an adenovirus model system.

GXP Program Details

Description of Program:

Lackland Air Force Base (LAFB) in San Antonio, Tex. is the location of Basic Military Training for all recruits to the United States Air Force. Approximately 40,000 basic military trainees (BMTs) undergo a 6-week training course prior to assignment of duty. These BMTs are organized into flights of 50-60 individuals that eat, sleep, and train in close quarters. Each flight is paired with a brother or sister flight with which there is increased contact due to co-localization for scheduled activities, and multiple flights are grouped into squadrons which reside in the same dormitory building, subdivided into dorms for individual flights. Compared with their civilian peers, young healthy adults serving in the U.S. Military are at a significantly elevated risk of respiratory infections. Crowding and numerous stressors facilitate the transmission of respiratory pathogens. During the 6-week basic training course, approximately 20% of BMTs will develop fever and respiratory symptoms.

Adenoviruses are the most common respiratory pathogens seen in the BMT population today. Before an adenoviral vaccine was available, adenovirus was consistently isolated in 30-70% of BMTs with acute respiratory disease. The outbreaks often incapacitate commands, halting the flow of new trainees through basic training. In 1971, the adenoviral vaccine directed against serotypes 4 and 7 became routinely available to new military trainees. This vaccine had a dramatic impact on trainee illness, reducing total respiratory disease by 50-60%, and reducing adenovirus-specific disease rates by 95-99%. The use of the adenoviral vaccine continued uninterrupted for 25 years until the manufacturer of the vaccine halted production. After discontinuation of the vaccine, 1814 of the 3413 (53%) throat cultures from symptomatic military trainees yielded adenovirus during the period from October 1996 to June 1998. At that time, adenovirus types 4, 7, 3, and 21 accounted for 57%, 25%, 9%, and 7% of the isolates, respectively, and currently a predominance of adenovirus type 4 is recognized. Since the discontinuation of the adenoviral vaccine, approximately 20% of BMTs develop symptoms of fever and respiratory illness and 60% of these cases are due to adenovirus. Other pathogens such as influenza A, Mycoplasma pneumoniae, Chlamydia pneumoniae, Bordetella pertussis, and Streptococcus pyogenes continue to cause a significant minority of respiratory disease in this population. Mixed infections are known to occur but the frequency and types of pathogens involved in mixed infections are largely uncharacterized. Resolution of mixed pathogens is the topic of a related patent application by the present group of inventors (U.S. Provisional Patent Application No. 60/590,931, filed on Jul. 2, 2004). In the present invention, the present inventors do not attempt to characterize multiple pathogens but rely on the predominance of a single pathogen (human Adenovirus type 4; Ad4) to create a category of infection and compare cases of that to other categories comprised of non-Ad4 FRI and convalescent Ad4 FRI.

With the current state of the art, differentiating the serotypes and strains of adenovirus and influenza is a time-consuming and labor-intensive undertaking. Cultures of adenovirus may take a week to grow and subsequent typing of the adenovirus isolate must then be performed using hemagglutination-inhibition and neutralization assays which are cumbersome and subject to significant reciprocal cross-reactions, making serotype identification take as long as 2-3 weeks. By the time that the virus is identified, the BMT has often has already transmitted the infection to multiple others. There is great need for more rapid diagnostic assays and a need to detail the epidemiology of these respiratory outbreaks so that public health measures can be directed appropriately.

More importantly, especially with regard to the present invention, there are no known methods to determine reliable physiological markers that relate the exposure of an individual to an infectious pathogen to the actual infection. Thus, while a sample such as a throat swab or nasal wash might produce nucleic acid markers for the presence of a respiratory pathogen, there are no techniques available to determine whether the individual will become ill or has just recovered from infection caused by that pathogen(s). In addition, an organism may be recovered from a sampling of the respiratory tract. Generally, it may be unclear whether this organism is simply colonizing the respiratory tract or is the cause of disease; assaying for the presence of an immunologic signature to this organism is expected to assist in the differentiation of colonization from disease. Furthermore, within the group of individuals who present with febrile respiratory illness, there are no methods for determining the severity of infection, or the degree and type of interaction with the host immune system. The present invention describes methods for performing these latter assessments in a statistically valid manner.

Entry Criteria and Sample Collection

In order to determine whether gene expression profiling could differentiate individuals infected and ill with adenovirus versus other infectious pathogens, the present inventors undertook an Institutional Review Board (IRB) approved study (vide infra). BMTs arriving at LAFB underwent informed consent to participate in this study. Approximately 15 ml of blood, filling 4 to 5 PAX tubes, were drawn from each volunteer. On day 1-3 of training, blood samples were drawn from healthy BMTs into PAX tubes by standard protocol (described herein elsewhere), but no nasal wash was collected for this group. A complete blood cell count (CBC) was also obtained. These individuals were determined to be healthy by screening with a standardized questionnaire, which eliminated any initial BMT with acute medical illness within 4 weeks of arriving at basic training.

In Phase II of the study, BMTs who presented at a later stage in training with a temperature greater than 100.4° F. and respiratory symptoms were consented for a nasal wash, throat swab and blood draw for PAX tubes and CBC. These individuals were categorized into either the febrile with- or without- adenovirus infection groups. At times, a rapid antigen capture assay for adenovirus was used to screen for individuals who were adenovirus negative; this was done to improve enrollment of individuals in this group. All results of rapid assay were confirmed with culture.

In Phase III of the study, approximately three weeks after sample collection from febrile volunteers with adenovirus, additional blood (PAX tube and CBC) and nasal wash were collected from these individuals when they recovered, forming the convalescent group.

All PAX tubes were maintained at room temperature for 2 hrs and then were frozen at −20° C. and shipped on dry-ice to the Navy Research Laboratory (NRL) in Washington, D.C. within 7 days for processing. Nasal washes were performed by standard protocol using 5 ml of normal saline to lavage the nasopharynx followed by collection of the eluent in a sterile container. Nasal wash eluent was stored at 4° C. for 1-24 hrs before being aliquoted and stored at −20° C. and shipped to NRL within 7 days for processing. The nasal wash and throat swab was sent for standard viral culture of adenovirus, influenza, parainfluenza 1, 2, and 3 and RSV. The nasal-wash and throat swab were also tested by a multiplex PCR for adenovirus type 4 to further confirm culture results for this pathogen. Although the foregoing describes the protocol undertaken in the present study, it is understood that the present invention further contemplates alternative storage and shipment conditions so long as the integrity of the sample is not compromised.

All BMTs underwent a standardized questionnaire at initial presentation, during presentation with illness, and at follow-up. Questions posed to BMTs include: vaccination history, allergies, last meal, last exercise, last injury, medication taken, smoking history, observed subjective symptoms, and last menstruation (if appropriate). Among the observed subjective symptoms asked and monitored are: sore throat, sinus congestion, cough (productive or non-productive), fever, chills, nausea, vomiting, diarrhea, malaise, body aches, runny nose, headache, pain w/deep breath, and rash. All data was stored in electronic format using personal identification numbers and date of sample collection.

During the period of sample collection, two outbreaks of Streptococcus pyogenes occurred. Throat swab and blood samples were collected as above on acutely ill BMTs and on those who recovered from illness and were still in basic training. Diagnosis of Streptococcus pyogenes was confirmed by bacterial culture and subsequently by PCR.

For the experiment supporting the present invention all male BMTs who were determined to be healthy (no acute medical illness in 4 weeks prior to initiation of basic training) were eligible for study. In Phase II, any male BMT with T>100.4 and respiratory symptoms were eligible for consent. In the experiments described in the examples below, the patient population enrolled consisted of male BMTs between the ages of 17-25. Seventy percent were white, 12% Hispanic, 12% black and 6% Asian. Thirty BMTs who were determined to be healthy were enrolled, 30 who had fever and respiratory symptoms and determined to have adenovirus by rapid assay (confirmed by viral culture and PCR) were enrolled, 19 with fever, respiratory symptoms and non-adenoviral infection were enrolled. The 30 BMTs with fever, respiratory symptoms and adenovirus had another nasal wash and blood draw performed during convalescence from their illness.

Metadata for the experiments supporting the present invention were obtained by providing the healthy incoming BMTs with a standardized questionnaire. These individuals were excluded from inclusion if they had fever, sinus congestion, nausea/vomiting, burning with urination, cough, sore throat, diarrhea or chills in the 4 weeks prior to basic training. In order to determine conditions that might affect baseline gene expression, these individuals were screened for: race/ethnicity, vaccination status, time of most recent meal, time of last exercise, perceived stress level, allergies, recent injuries, current medications, and smoking history.

For Phase II, when BMTs were presenting with fever and respiratory symptoms, a standardized questionnaire was administered. In order to determine conditions that might affect baseline gene expression, these individuals were screened for: race/ethnicity, vaccination status, time of most recent meal, time of last exercise, perceived stress level, allergies, recent injuries, current medications, and smoking history. The duration and type of respiratory symptoms to include sore throat, sinus congestion, cough, fever, chills, nausea, vomiting, diarrhea, fatigue, body aches, runny nose, headache, chest pain and rash were recorded on standardized forms. A physical examination was recorded on standardized form to detail signs of illness in the BMT. Type and duration of medications taken were recorded.

For Phase III when the BMT with adenoviral illness had recovered (14-28 days after presenting ill) another standardized questionnaire was administered, including questions on time of most recent meal, time of last exercise, perceived stress level, allergies, recent injuries, current medications, and smoking history. The total duration of each symptom from the Phase II questionnaire was noted and the total period of recovery from each symptom was determined. A detailed history of medication use between the time of Phase II and Phase III was taken.

The ability to collect samples in a longitudinal study enables one to study gene expression throughout the course of an infectious illness. In a study as outlined hereinabove and further supported by the examples of the present application, the present inventors particularly followed BMTs who were ill with adenovirus through the time of their recovery from disease. The detailed database on type and duration of symptoms thus enabled the present inventors to determine whether these factors impact the gene expression signature for adenovirus and Streptococcus pyogenes. Further, the detailed database also enabled the present inventors to discriminate early versus late disease and the severity of disease (for example, expected duration of illness/symptoms).

The detailed and standardized collection of information such as recent meal, recent exercise, perceived stress level, recent injuries, current medications, and smoking history enable control of confounding variables, strengthening the conclusion that identified gene expression patterns are specific immunologic signatures of particular pathogens. This collected information also can be used to determine whether such conditions significantly impact gene expression patterns in a population. A statistical assessment of whether these factors are necessary or confounding for correct classification will determine whether it will be necessary to monitor for them in future experiments and applications.

In the future, gene expression patterns (immunologic signatures) for particular pathogens at different stages of disease may be used to predict morbidity and mortality. This may assist the healthcare professional in determining the appropriate level of care (type of medications to use, level of care required—admit to hospital or provide care in the outpatient setting). There currently are algorithms for determining whether individuals with respiratory infection (particularly pneumonia) should be admitted to the hospital (and to what level of care) and these algorithms rely on such factors as degree of fever, heart rate, respiratory rate, blood gases and blood chemistries (47, 48) (49). A detailed understanding of the state of immunologic activation of the ill individual through gene expression may further assist with determining severity of illness.

Moreover, understanding gene expression patterns, based on the inventive techniques herein, in individuals who are recovered from a particular infectious illness would enable forensic analysis of past outbreaks. Subsequently, this information may be used to determine whether certain pathogens are naturally endemic in specific geographic areas or whether new infections have been imported to regions (e.g., how many have been previously infected with West Nile Virus?).

Further, for an individual, the present invention enables determination of whether these individuals have been infected with a particular infectious pathogen in the past and from this information determines the likelihood of immunity/protection against future infection with the same or related organism. Such information would be valuable as it could guide whether vaccination or prophylaxis is necessary for particular deploying/deployed troops or hospital workers.

Assessment of Use of PAX Tubes in “Real World” Scenario

Having established a prospective, longitudinal study using PAX tubes, this gave the present inventors the opportunity to assess the quality of the modified protocol for gene-expression analysis of RNA using PAX tubes and the Affymetrix Genechip platform in a real world test bed of ongoing epidemics of upper respiratory disease.

Many factors contribute to the variability of target detection, with the quality of RNA being one of the most important. The quality of RNA from PAX tubes collected blood could be influenced by the disease status of the donors, sample handling, and other downstream processes. Previously, the present inventors showed that under two conditions representative of practical sample handling, the PAX system was capable of preserving blood RNA to produce good quality metrics and relatively stable transcriptome measurements (50). Recently, new RNA quality metrics have been proposed based on associations between experimental treatment of cells or purified RNA to induce RNA degradation and metrics derived from electropherograms of the RNA on the bioanalyzer (51). One new metric is the degradation factor (% Dgr/18S), which is the ratio of the average intensity of bands from degraded RNA, that is peaks of lesser molecular weight than the 18S ribosomal peak, to the 18S band intensity multiplied by 100. It is a continuous variable that is used to derive a categorical variable named ‘Alert’. Alert has five values:

BLACK—indicating that the ribosomal peaks were not detected;

NULL—no RNA degradation and corresponds to degradation factor values □8;

YELLOW—for RNA degradation can be detected and values from >8 to 16;

ORANGE—for severe degradation and values from >16 to 24;

RED—for highest alert, strong degradation, for values from >24.

Another new metric is the apoptosis factor (28S/18S), which is the ratio of the height of the 28S to 18S peak and is indicative of the percentage of cells undergoing apoptosis (51). The present inventors compared the RNA QC methods of electropherograms from the Agilent 2100 bioanalyzer, the degradation factor, Alert, and the apoptosis factor to determine which is the best indicator of sample processing quality for RNA used in microarray gene expression analysis.

Thus, for PAX system isolated RNA from the present inventors previous study (50) and current BMTs cohort, the distributions of RNA quality metrics were reported, which would be useful for comparisons and planning of protocols by other labs; determined the up-stream quality metrics that are most indicative of the quality of microarray target detection outcomes; and determined the effects of inter-individual hemoglobin variability on the sensitivity of target detection.

The present inventors demonstrate that the Alert metric was a robust indicator of microarray results and will be useful for high throughput RNA quality control, especially as one practically cannot look at all the electropherograms directly during an ongoing study and must be able to rely on an indicator to flag a sample for further evaluations.

The magnitude of the apoptosis factor suggested that a high percentage of blood cells underwent apoptotic cell death. This could be due to the PAX RNA stabilizing reagent inducing cell death via apoptosis upon contact with blood cells, or simply due to differences between whole blood and cultured cells from which the apoptosis factor was derived. If interested in studying apoptosis related pathways, one would have to investigate this property further with the PAX system technology. In this manner it may be possible to correlate the apoptosis factor with gene-expression profiles to implicate apoptotic pathways.

The stability of the RNA from PAX tube blood that was handled a variety of ways suggest that for future studies one can be more confident in the stability of RNA throughout the range of these handling conditions.

The present inventors were next able to explore appropriate methods of scaling of gene expression arrays when applied to detection of clinical phenotypes. While global scaling approaches have been advocated for other study designs and uses involving gene expression arrays, we concluded that the use of the 100 housekeeping genes provided the least biased approach, although 5 approaches were considered:
1) double scaled global normalization
2) no normalization at all
3) 100 hk gene scaling
4) 100 hk gene median normalization
5) empirical set of normalization gene

After QC/QA of the PAX tube RNA and the microarray scaling, we undertook class prediction and class comparison modeling (a summary appears in Tables 7, 10, and 11). The class prediction using gene-expression, suggestively, performed better than using CBC or electropherograms alone. This could be that gene-expression does in fact contain more information about the sample or that it simply has more variables thus providing more opportunities to find a good classifier by chance alone. More specifically, the p-value for the significance test of classification rate suggests that gene expression is better for classification than the CBC or electropherogram and that it is not likely a function of number of variables acquired because the CBC actually has 10 times as many as gene expression and performed poorly.

Study to Increase Number of Pathogens Recovered (the Hospital Study)

In order to study another patient population (broader age range, male and female, civilian) and to increase the number of pathogens recovered, another protocol was undertaken which focused on patients presenting to medical clinics and hospital wards at the Wilford Hall Medical Center at Lackland AFB (sometimes referred to herein as “the Hospital study”).

For the Hospital study, patient selection (Inclusion criteria) was conducted as follows. Adults (male and female) greater than the age of 18 were included. All were presenting to the hospital or hospital clinics with temperature >100.4° F. and respiratory symptoms. Nasal wash and throat swab were collected most commonly by a study nurse or by medical personnel who had been instructed by the study nurse. A portion of the nasal wash was used to screen for influenza A or B by rapid antigen capture assay (52) and this result was confirmed by culture and PCR. All nasal wash specimens were additionally cultured for Parainfluenza 1, 2, 3, RSV and adenovirus. Accordingly, in an embodiment of the present invention, the gene expression analysis may be combined with one or more pre-screening methods. For example, the pre-screening method may include abovementioned influenza A or B rapid antigen capture assay, a culture assay, a PCR-based assay, a method described in U.S. 60/590,931, filed on Jul. 2, 2004 (the entire contents of which are incorporated herein by reference).

A CBC will be obtained for all enrollees with differential. In addition, each enrollee will be given a standardized questionnaire including questions relating to race/ethnicity, vaccination status, time of most recent meal, time of last exercise, perceived stress level, allergies, recent injuries, current medications, and smoking history. The duration and type of respiratory symptoms to include sore throat, sinus congestion, cough, fever, chills, nausea, vomiting, diarrhea, fatigue, body aches, runny nose, headache, chest pain and rash are recorded on standardized forms. Physical examination findings are recorded on standardized forms.

This is a cross-sectional study that includes adults of all ages with differing severity of disease (some will be in the outpatient clinic setting and others admitted to the hospital). The ability to collect blood samples over more than one influenza season will enable the present inventors to determine the gene expression pattern to influenza A and B and may allow us to determine whether there is a specific gene expression pattern for different strains of influenza A (H1N1 vs. H3N2).

For this study, the present inventors will monitor whether individuals received the injectable form of the influenza vaccine and the timing of vaccine relative to illness. The present inventors will discern whether the gene expression pattern differs between individuals with “breakthrough” influenza-illness occurring greater than 2 weeks after time of influenza vaccine compared to the gene expression pattern seen in unvaccinated individuals with illness. The present inventors will perform the same comparison for those individuals who receive FluMist (MedImmune Vaccines) intranasal vaccination with a live, attenuated strain of influenza. Understanding gene expression patterns after vaccination may predict likelihood of protection from disease and likelihood of breakthrough illness; the efficacy of the influenza vaccine is considered to be 70-80%

Because the Lackland BMT population will be receiving FluMist as a strategy of prophylaxis during the 2004-2005 flu season, the present inventors will assess gene expression profiles in individuals who receive FluMist and develop flu-like symptoms and those without in the 7 days following vaccination; it is well know that individuals receiving FluMist may develop cough, sore throat and muscle aches in 2-7 days post-vaccination as they shed the attenuated virus (CID 2004:38 (1 March), 760-762 full reference below), but the gene expression pattern post vaccination has not been determined. This study will allow us to determine whether there is a gene expression pattern that enables us to differentiate which individual is symptomatic after FluMist vaccination, but developing a protective immune response and which individual has actually developed cough, sore throat, muscle aches due to acquisition of circulating wild type influenza in the population. This is a critical distinction to make in a closed population, such as the BMTs or college students in dormitories, because it is this age group that is most appropriate to receive the FluMist vaccine and yet the most likely to have transmission of wild type influenza in closed quarters.

Presymptomatic Study

Individuals typically become infected with an infectious pathogen and remain asymptomatic during the incubation period prior to onset of disease. During this incubation period, the host begins to mount an immune response to the infecting pathogen. Typically the initial response is the innate immune response mounted by natural killer cells and neutrophils. Later in infection, the specific host immune response comprised of T lymphocyte, B lymphocyte and antibody responses becomes effective. In some infections, such as with the bioagent, Francisella tularensis, as few as 10 organisms can ultimately cause symptomatic disease; while this small number of organisms can be difficult to detect directly, the host immune response typically constitutes an amplified response of literally millions of immune cells and this immunologic signature can likely be detected prior to the onset of clinical symptoms.

There are clinical scenarios in which it would be advantageous to the health care provider, public health officers and commanders/public officials to determine not only who is infected with a particular pathogen, but who has also been exposed to this same pathogen either by direct exposure or through transmission from an infected index case. For example, if the infectious agent of smallpox was released and an index case was detected, it is anticipated that each index case would significantly expose close contacts (face-to-face contact within 3 feet) via respiratory droplets and nuclei. Typically, for each index case of smallpox as many as 10 other susceptible individuals may develop the disease. In view of the limited amount of smallpox vaccine and potential adverse reactions to the vaccine, predicting who amongst the exposed would develop disease could direct resources and limit adverse side effects of the vaccine. Gene expression studies can detect developing, specific immunologic signatures for pathogens and assist in determining who in a population has been significantly exposed and infected (carrying organism) and who amongst the exposed-infected will ultimately develop disease. Therefore, the methods of the present invention are particularly useful for the identification of gene expression signatures and the results obtained thereby may be used directly to guide and/or tailor therapeutic regimens.

To this end, the following study design permits the study of cues and expression profiles at various stages of pathogen exposure and onset. Since the majority of BMTs arriving to basic training from their respective home communities will be susceptible to infection with adenovirus, the present inventors are able to screen BMTs presenting with fever and respiratory symptoms to Lackland AFB clinics with a rapid assay for adenovirus. Once a BMT is identified as being infected with adenovirus, the BMTs with whom he/she has had face-to-face contact can be followed for infection and subsequent development of disease. Significantly exposed BMTs can have blood drawn for gene expression during the exposed/asymptomatic period and again after development of disease and during recovery. Gene expression patterns obtained from these time points are then analyzed to determine the gene expression pattern that best predicts development of disease.

In anticipation of the abovementioned study, BMTs who are ill with fever and respiratory symptoms during basic training are receiving a standardized questionnaire to determine other BMTs with whom they have had face-to-face contact within the last week; a database is being generated which labels the infected BMT as the current “index case” and all BMTs with who he/she has had recent contact as “exposed”. Data on the exposed and their relationship to the index case are maintained; for example, the exposed may have been the Training Instructor or Dorm Chief or Element Leader of the index case. If an exposed case next presents to a clinic with fever and respiratory illness, then that case is linked to the initial index case as well as to other BMTs to which he/she may now have exposed. The epidemiology is followed to determine whether there are situations in which the infectious respiratory disease is most likely transmitted; i.e., do Dorm Chief or Element Leaders most commonly transmit to individuals within their dorms or elements? This will direct the EOS clinical team on who constitutes the best case definition for “significant exposure” and, thus, which BMTs would be best to draw for gene expression studies in the “exposed” group. This group will be followed for subsequent development of disease and blood will be drawn if these individuals present with fever and respiratory symptoms.

Next the present inventors describe the present invention in terms of GXP Protocols and Data handling

Description of Transcriptome/mRNA Measurement Techniques:

There are several techniques to quantitatively measure mRNA at various level of throughput. Some of them are Northern blot, RT-PCR, Nuclease protection assay, Quantigene, SAGE, differential display, in situ hybridization, nanoarrays and microarrays. Some of these are not readily adapted for high throughput or can measure at the transcriptome level. For our purposes of surveillance and biomarker discovery, microarray based techniques are most amendable for these purposes. Once biomarkers are discovered, techniques that have short processing time, but less parallel processing capability may be more useful for diagnostic purposes, such as RT-PCR or Quantigene. Techniques to measure mRNA generally involves sample preparation, mRNA amplification and labeling if needed, followed by hybridization, then washing, staining, and/or detection of signals. There are variations to all these major steps. Sample preparation may be extensive such as for the Affymetrix genechip platform or minimal such as the Quantigene system from Genospectra. Ideally, for our purpose, we want to measure the most number of transcripts in the shortest time and the highest sensitivity and specificity. Although we have used the Genechip technology to discover biomarkers and pathways, there are many possible improvements on the current Affymetrix technology or other technologies that one can think of or already available to assess in the field (several of which are discussed herein and form a part of the present invention).

Improvements Over Standard Microarray Techniques:

For the platform that the present inventors have tested, the Affymetrix genechip platforms, recent improvements include reducing the amount of initial RNA needed, shortened time of processing, or robotics to facilitate high throughput and reduce operator variability. Several options are available on the market to incorporate into the sample processing step of the Genechip platform. One is the new IVT kit from Affymetrix that can use 1 μg starting amount of total RNA versus 5 μg previously. Another is the double cycle IVT from Affymetrix that can start with 10 ng total RNA, however, the processing time and complexity of the assayed is increased. The Ovation kit can also amplify and label RNA starting with as low as 5 ng, and they claim the time is in 4 hours. However, it has not been extensively tested with the Genechip microarray. A recent publication also attempted to label the mRNA directly without amplification to shorten processing time, but the sensitivity was reduced.

There are many areas of improvements at various steps in the processing that the present inventors contemplate in the present invention. One is to combine and develop various steps in the surveillance process. For sample collection, instead of Paxgene, one could use microcapillary tubes to collect blood, then stabilize with RNAstat, then isolate RNA via several available kits for RNA isolation from small volumes of blood, such as the Dynabeads® mRNA DIRECTTM Kit that can isolate mRNA using only 1 tube in 15 min, then use the Ovation kit to amplify and label, followed by hybridization onto Genechip and wash and stain the next day. In addition, the hybridization time may be reduced from it current time of 16 hrs on the Genechip to a time ranging from 8-14 hours, preferably 10-12 hours, or even shorter times. To further reduce the hybridization time, the present invention contemplates applying a strong electric/magnetic field to the chip during hybridization. Also to reduce hybridization time, the hybridizing temperature may be increased and then ramp down to 45° C., the current temperature for hybridization.

To improve sensitivity, the skilled artisan may employ alternative signal emitters. Currently, the signal emitter is the strepavidin-phycoerythrin followed by further amplification with biotinylated anti-strepavidin. However, the present invention contemplates the use of the branch DNA from Genospectra to amplify signal, quantum dots followed by multiple scans as the quantum dots do not quench, alexi dyes, or biotin labeled viruses which greatly increase signals because of reduced quenching, higher quantum yields and up to 120 biotin molecule per virus, or RLS particles. Even further, the present invention contemplates the use of probes that are synthesized onto a conductive material, thereby it is possible to detect via electrical signals upon duplex formation, and then one can detect signals right away. In even a further embodiment, another mRNA measurement technology may be employed altogether, especially a nanoarray developed to measure mRNA from single cells.

Data Acquisition:

In the present invention data acquisition is performed using scanner (genechip) and computer.

Data Handling and Analysis:

Data acquisition and handling may be performed by any means known by the skilled artisan. For example, data acquisition and handling may be performed by hand and passing through various programs. The present inventors are in the process of developing software to perform all necessary data analysis automatically and provide results.

Algorithms for Metadata and Microarray Parsing, Grouping, etc.:

Pseudocode: Genes are ranked by likelihood to discriminate
Binary vs. multi-characteristic classifiers. Binary classifiers form binary trees to classify clinical phenotypes into groups. Each node of the binary tree is determined by the minimal percent misclassification. The result is that at the tip of each tree should be each group of phenotypes; although some phenotypes may not always be able to be segregated because of lack of classifiers discovered. A multi-characteristic classifier immediately sorts out the phenotypes instead of dividing through a tree. Both methods are currently methods of research. The present inventors' results so far suggest that for a mixture of phenotypes with large and small optimal classifiers, the binary method may make more sense. For instance for distinguishing the healthy and sick, one can obtained a relatively large number of genes in the classifier, whereas for distinguishing sick with adenovirus and sick without adenovirus, only a relatively small number of genes in the classifier may be found. The present inventors' example analysis of the gxp class prediction is basically a binary analysis with comparisons between nonfebriles vs. febriles, then healthy vs. convalescents, then febriles with adenovirus vs. without. This is basically a manual version of binary class prediction. A multi-characteristic classifier would classify healthy, convalescent, febriles with, and febriles without adenovirus all at once, without going through binary nodes. The current ArrayTools software can only implement binary tree classification with equal univariate alpha parameters for all tree nodes resulting in large classifiers for the first node, and smaller ones for subsequent nodes for our gxp data. One possible future method is to allow for different univariate alphas at each node to equalize the size of the classifiers for each node. Binary tree methods are also very computationally intensive, especially for finding p-values of misclassification rate. One needs to perform further in silicon experiments to find the best algorithm for class prediction especially where the dynamic range of differences among classes vary greatly, as in our case. For binary classification, one can also consider different information from outside non-gene-expression assays to include at each node in deciding which branch the case shall be classified. Based on our current gxp results described herein, the data could be classified into the four groups with less than 50 genes at each binary node at a certain percent accuracy at a certain probability of certainty.
Full Analysis of gene expression data: For analysis of the GXP results from the N=30 study, first, normalization of complete cell count data, electropherogram data, and gene-expression data was carried out after considering various methods. Then, data quality was assessed via individual control charts to determine measurement process stability, outliers, and comparisons to standards suggested by Affymetrix or from other laboratories. This quality control results in a set of reliable samples for analysis. Then RNA quality from pax tubes is assessed via overlaying graph of electropherograms and RNA quality metrics. And the relationship between RNA quality variability and microarray variability is determined. Once quality and reliability is established, then filtering parameters are set to reduce number of variables. Then, class prediction analysis using supervised methods was performed and optimized to determine sets of genes that could classify clinical phenotypes at a certain percent accuracy with a certain reliability using permutation tests. Potential confounders for clinical phenotypes are also assessed to assure that the classifier genes are most likely due to clinical phenotypes rather than confounders. Then, class comparisons analysis is carried out to determine genes that show differences between clinical phenotypes. Finally, functional analysis is carried out to determine pathways involved in disease phenotypes. Many more analysis can to performed, such as gene ontology comparisons, promoter analysis, genome distribution, variation of immune responses in the population, modeling of differential gene expression while controlling for cell count heterogeneity, and comparisons with public microarray databases, and cross platform analysis, discover functions of genes with unknown functions.
Diagnostic Capability: This is assessed by determining sensitivity, specificity, positive predictive values, negative predictive values of the assay. Some of the sensitivity and specificity of the class prediction for the gxp study has been calculated as described herein. Overall, the goal is to optimize the ROC curve of class prediction results, which is analogous to minimizing the misclassification rate. Negative and positive predicted values can be calculated once the prevalence of a disease is known. Improving assaying time, sensitivity, reliability, and automation of the assay and analysis would further facilitate diagnostic capability. To this end, once ethical issues are resolved, the human implanted chips to connect a patient to medical histories would aid in automated analysis and prediction of disease outcomes. The utility of gene-expression data for many diseases also greatly enhances diagnostic capability. Linkage to genomic variations would also provide much medical prognosis of patient. Also advancement of gene-expression technologies to nano scaled microarrays should greatly enhance diagnostic potential. For the gxp study exemplified herein, the diagnostic classifiers will be validated with a larger prediction set; however, even with the data set supporting the examples of the present invention, this can be assessed. For the minimal classifiers of healthy versus fever, the prediction set was 100% accurate regardless of processing differences from the training set. But processing differences in measuring gene expression has a greater effect on classes with less different phenotypes, such as among the sick alone. Further analysis study into the effect of the number of genes in classifiers on class prediction results of the prediction will be assessed. Future prospective studies will more assuredly assess the diagnostic capability of the classifiers we have found and began to validate in the gxp study.
   GXP for Prognostic Ability
Experimental Protocol
   Baseline Patient and Track Through Disease Onset
      In order to determine the prognostic capability of gene expression for prediction of disease timing, severity and response to treatment, one must have a cohort that can be followed from healthy status through infectious exposure to disease/symptom onset. The Lackland BMT population is unique in that this population has ongoing, significant endemic rates of upper respiratory disease with frequent epidemic rates. This enables studies to determine gene expression markers in pre-symptomatic individuals. An index case with a specific febrile respiratory disease will be identified and those BMTs significantly exposed will be assayed for gene expression to determine the immunologic signature that predicts later development of disease. BMTs with disease will be followed to assess severity of disease and relationship to gene expression.
   Challenge with biologically hostile environment
      BMTs who are naturally exposed and infected with a biological agent, such as adenovirus, will be assayed for gene expression. This group may or may not subsequently develop disease and the comparison of gene expression profiles will be made between the groups.
Opportunity to track genes as function of time and disorder
Prognosis relating to a) propensity to become ill, b) timeline to onset of disorder, c) efficacy of treatment regimen, d) recovery, etc.

Ability to Validate Diagnostic and Prognostic Methods and Classifiers
Rationale and Methodology

To validate diagnostic and prognostic methods and classifiers. First the present inventors performed an experiment to discover classifiers for certain diseases and/or phenotypes. Then, the percent correct classification is optimized by varying various methods and parameters. These classifiers are validated at this stage via leave a subset of samples out cross validation methods. Also, the reliability of the optimal percent correct classification using the discovered classifiers is assessed via the permutation test. Once the optimal classifier and algorithm is found and validated with the training set, then additional samples are collected and measure to form the prediction set. The optimal classifier and algorithm is used to classify cases in the prediction set to further validate the classifiers because the prediction set is completely independent of the training set which was used to discover the classifier genes and to validate them statistically. Additionally, the classifiers are further validated using different assaying methodologies, such as RT-PCR, to further confirm that the classifier gene set is biologically significant and not simply assaying mythology specific. Then the classifiers are tested further in a larger sample of the population for which the assay is intended to be used.
The present method permits detection of independent gene signatures for virtually any microorganisms. Notable examples include:
   Influenza: Influenza A and B immunologic markers will be determined to both naturally-occurring disease as well as vaccine induced immunity (both intramuscular and intranasal vaccination).
   Streptococcus Pyogenes: Ongoing studies are assessing the gene expression biomarkers for S. pyogenes in the BMT and clinic population.
   Ad4: Currently we have identified gene expression biomarkers distinguishing febrile adenovirus positive patients from adenovirus negative patients.
   Additional microbial infections include those caused by Adenovirus species, N. meningitides, Influenza A and B, Bordetella pertussis, Parainfluenza I, II, III, S. pneumoniae, Rhinovirus, C. pneumoniae, RSV, S. pyogenes, West Nile Virus, B. anthracis, Coronavirus, Variola major, Ebola virus, Lassa virus, F. tularensis, Y. pestis
Combinations of disorders
   Additionally, gene-expression of the host indicates functional bioactivity of a subset of agents among a set of agents challenging the body. Thus, results from host gene expression should synergized with results from other assays that measure only pathogen genomes, such as PCR, RPM, or chembioagent antigens, such as immunoassays. Because of current highly parallel usage of these assays, often one gets multiple results, such as indication of multiple infection in the presence of asymtopmatic infection, where it is not clear which agent is the causative agent. Gene-expression profiles may provide information to sort this out. Also, for multiple etiologic agents inducing similar diseases, the results from gene-expression profiles may be analyzed for common nodal pathways with high connectivity, which then can be targeted as treatments intervention via therapeutics such as drugs. This would also suggest usage of therapeutics that is known to target a pathway for a particular disease to other diseases that activate the same pathway.

The present invention also offers the practitioner and clinician an ability to monitor and/or validate expression profiles identified by other assays. For example, the Griffiths et al (71) report biomarkers for malaria determined by monitoring host gene expression in whole blood from patients suffering from acute malaria or other febrile illnesses. Cobb et al (72) report the effect of traumatic injury upon the gene expression profile of blood leukocytes. While Rubins et al (73) report the gene expression profile determined for primates suffering from smallpox. The methods of the present invention can be used to assess the accuracy and reliability of the biomarkers identified in these, and similar, and to determine whether these biomarkers can be utilized to trace disease progression.

Exploiting Prior Acquired Knowledge (Bayesian Priors)

Recognition of Signature (Host Response Chip)
   In this method, the present invention may be combined with other diagnosis methods (i.e., RPM, standard blood test, immunoassay, etc.) to enhance accuracy of diagnosis. Diagnosing the health status of an individual and prognosing their course of disease usually require several assays ranging from assessment of signs and symptoms to laboratory diagnostic tests. Each assaying provides a pretest probability of positive and negative predictive values for the next assay. Bayesian statistical theory takes into account this pre-test probability (whether subjectively determined or via an assay) to determine the predictive values of the subsequent test, which should provide more accurate information to help the clinician in discerning course of action. An example of this is the present inventors' analysis of class prediction based on the Complete Blood cell count (CBC) and then the electropherogram data, and then the gene expression data. Although these different assays are not what the clinician normally use for class prediction of disease, the statistical analysis illustrates that the gene-expression profiles provided the highest amount of accuracy for prediction of infection status. If binary class prediction algorithms are considered, than for each node in the binary tree, one might consider diagnostic and prognostic probabilities from other established assays in addition to the gene-expression biomarker assays which likely will provide the most information for better diagnosis and prognosis.
      Questions and hypotheses that may be explored with the database approach developed by the present invention

In addition to determining the gene expression profiles in response to pathogen exposure, there are many more questions and hypotheses that could be explored with the database developed by the present inventors. Some of these questions are listed below:
1) Can one find classifiers for clinical subtypes, such as those who are febrile and negative for adenovirus by culture, put positive by PCR? There are some discordances between infection status as determined by assay type, such as culturing, PCR, or pathogen microarray. Can one use gene-expression data to classify these discordances?
2) What are the concordance, sensitivity, and specificity relationships between these culture, PCR, and gene-expression classification?
3) Is there a circadian rhythm relationship between time of PAX tube collection and certain genes in the expression profiles? Gene expression profiles that correlate with time of day should relate to circadian rhythm functions
4) Do lot numbers affect anything?
5) How do different statistical models to determine transcripts abundance compare to current results? There are multiple models for determining the quantity of transcripts based on amount of light emitted from each cell for each probe. Some of these are Mas4 algorithm, MAS 5 algorithm, and multi-chip models: RMA, dChip, Plier, and mix models. The GXP results herein suggest that one cannot use the multi-chip models because those models usually assumes relatively small changes in gene expression profiles between experimental groups, which is definitely not the case in surveillance studies of multiple disease states.
6) How will different normalization algorithms compare to current results? There are many normalization methods: median scaling, trimmean scaling, quantile, splines, and others. Generally, we cannot use any normalization method that assumes that the distribution of the gene expression profiles is generally the same for groups such as healthy vs. sick. Thus the present inventors have found from the current study, that spiking in polyA RNA would be most logical for normalization for quantitative comparisons among samples.
7) How will we reduce the dimension of the data? (Principle Component Analysis, Singular Value Decomposition, robust Singular Value Decomposition?) This analysis will give an idea of how many independent components explain the majority of variation in the gene expression data.
8) What is the variation structure of the data and which of the metadata variables contribute most to the variation? Which contribute least?
9) Which of the component of the variation structure of the data classify certain metadata variables most accurately?
10) What is the latest in gene expression analysis from the literature? Can we use any of these new methods and/or software?
11) Are there subgroups in the adenovirus negative sick population? The adenovirus negative sick population can be due to multiple agents. Can evidence for this be found in the data set obtained by the present inventive methods?
12) What is the difference between poly A and total RNA samples?
14) What are the functions of the genes found to be involved in classifying the different phenotypes?
15) For the normal group especially, what is the variation of gene-expression for genes that are biologically equal in expression in the cohort? What genes show more variation among individuals than background variation?
16) Is there more than normal variation in immune related genes in the cohort? How many types of immune responses are there to virus infection? Is there a Th1 versus Th2 response?
17) Do genes that show high variation in expression correlate with variations in DNA sequences?
18) Is there a clustering of gene locations on the chromosomes for genes that differ among phenotypes?
19) Is there a high occurrence of certain promoter sequences for the genes that changed?
20) Further investigation of the pathways adenovirus infection and fever? What does this imply about the biological mechanism of adenovirus infection and fever in humans?
21) Can we confirm differences in these genes with RT-PCR? What is the percentage of concordance?
22) How do the genes that we found relevant in our study compare with published in vitro study of adenovirus infection? Other virus infection? Other phenotypes such as Smoking exposure?
23) Use genes that are cell type specific to decipher whether our gene list is associated with certain cell type differences
24) Can we do cross platform and/or lab analysis?
25) How do the different published methods for low level analysis, unsupervised and supervised clustering, and others compare with our data as oppose to cancer data?
26) Can we come up with better models?
27) Can one come up with a statistical model determine differential gene expression at the per cell level for groups with differing CBC?
28) What are the genes correlating with other quantitative traits recorded? Such as time of last meal, exercise, etc. These genes may be able to be used for determining the activity of a person at some previous time at a certain probability level.
29) Once pathways involved in fever are determined, one maybe able to find genes involved with less variability across the population than others. This may imply that these genes should be targets of drug development with effects that would be more efficacious for the-population. Whereas pathways with genes that show high variation across the population imply these genes may not be good targets for drugs intended for the general population.
   Application to Normal Gene Expression Measurement

The present invention will certainly find application in the measurement of “baseline” (i.e. normal) gene expression signature measurement. This would have great value in defining the establishment of baseline gene expression profiles across defined demographic populations. Such baseline measurements would have high value in discovery of fundamental differences between sexes, races, and the development and ageing processes. The value of such population gene expression profiling is illustrated in the phenomena such as Gulf War Illness following putative exposures to chemical weapons and environmental toxins wherein a variety of immune disorders were reported (53, 54) without the identification of a specific etiology. In response to Gulf War Illness, the Department of Defense initiated a broad baseline study known as the Millennium Cohort that has collected general health questionnaires from hundreds of thousands of active duty military personnel in hopes of establishing “baseline” indices of normal health. In contrast, baseline gene expression for 10⁵ to 10⁶ specific 25-mer transcriptional sequences would provide orders of magnitude greater information regarding the possible genomic and physiological etiologies of phenotypic or asymptomatic illnesses caused by external perturbations.

Application to Diagnosis Other Blood Disorders and Disease

The present invention may also be used for diagnoses of: oncology diseases including: CML (bcr/ab10) (30), circulating tumor cell detection, colorectal cancer-recurrence, neurology (MS), hemostatus and thrombosis, inflammatory disease (48 inflammatory genes for Rheumatoid Arthritis from Source Precision Medicine), diabetes, respiratory disease, and cytotoxicity and toxicology. (55). Generally, the present invention may find utility in any diseases or physiological states that have mRNA biomarkers from blood can use similar methods described herein.

Pre-Symptomatic Prognosis and Assessment of Disease Risk

Although it has been speculated that gene expression profiles could be diagnostic for asymptomatic disease diagnosis and prognosis, the practical reduction of that concept to practice has proven quite elusive. At least one prior study has shown that peripheral blood leukocytes obtained using PAXgene kits has yielded evidence of the utility of obtaining cDNA microarray baseline (i.e. healthy) expression signatures (Whitney et al 2003) (18). Other studies and prior art have shown time exposure of a known dosage of an infectious agent can lead to detectable signatures.

However, it has been exceptionally difficult, if not impossible to obtain experimental cohorts that allow simultaneous measurement of gene expression profiles in a homogeneous, isolated and experimentally accessible human population that contains statistically significant numbers of the following categories: (1) healthy baseline individuals in the identical physical environment as those who will be infected with a pathogen, (2) individuals who do not have an acquired immunity against a pathogen but encounter a low level of pathogen exposure to that pathogen, or have a high innate immunity, and exhibit distinguishable “successful” immune responses against the pathogen and do not become symptomatic for illness, (3) individuals who become ill following actual pathogen exposure and manifest symptoms without becoming febrile, (4) individuals who are exposed to the pathogen and develop illness with symptoms satisfying criteria for “febrile respiratory ill” (FRI) but who do not become so ill as to require hospitalization, (5) same as 4 except that severe illness develops and the individual meets medical criteria for hospitalization, and (6) individuals in various stages of recovery from categories 3-5.

While individuals are incubating an infectious agent and before the onset of symptoms, the innate immune system begins to mount a rudimentary response followed by a more effective specific immune response. During these phases, immune cells manufacture various cytokines and chemokines to mount an effective response. These biomarkers of the immune response provide an immunologic signature that may precede clinical symptoms.

Thus, there is a critical need to develop methods for discovery of unique gene expression patterns for various time points within the above mentioned classes, and the present invention successfully demonstrates those methods.

Preferred Uses of Pre-Symptomatic Assays Based on Gene Expression Profiles

Assays for pre-symptomatic diagnosis and prognosis of infectious disease would find utility in a variety of applications where the information is of sufficient quality to provide decision-quality information. For example, individuals who are at risk to themselves, to others, or to the completion of an important task as a result of probable or imminent illness can be temporarily replaced until the impending illness is managed. Examples would include pilots (commercial or military) prior to long-range flights, surgeons, etc.

Another use would be in the mitigation of an act of bioterrorism or industrial accident where hundreds, thousands, or even millions of individuals would be exposed to varying degrees of a toxic or infectious agent. Data obtained following the 2001 anthrax attacks in Washington, D.C. and New York, N.Y. indicated that for every 1 person who obtained a sufficient exposure to anthrax cause illness and death, there were another 1,500 “worried well” persons who were candidates for prophylactic administration of antibiotics. This number could have been orders of magnitude higher if the agent had been infectious (e.g. smallpox virus) instead of anthrax. If the remedial action, such as the administration of a high dosage of vaccine, antibiotic, or drug carries an associated risk (e.g. highly adverse reaction in 1 out of every 250 persons) then the remedial action could be of greater threat to public health than the initial attack or accident without the appropriate assessment of risk within an exposed population. Alternatively, the vaccine, antibiotic, or drug may be in short supply and a triaging of exposed individuals would be highly desirable to make maximal use of available quantities. Thus, a set of pre-symptomatic indicators could be of critical importance in the appropriate application of countermeasures in the above-mentioned situations.

Alternative Methods and Platforms for Detection of Transcriptional Markers

In the above-mentioned applications, it will be necessary to measure specific sets of transcriptional markers in a more rapid and cost-effective manner than that using a DNA microarray. Thus, the high density DNA microarray is a high-content discovery tool that teaches the distillation of the most meaningful transcriptional markers. Although, recent advances, such as shortening time of sample and target preparation with small initial amounts of RNA may allow the high density DNA microarray to be a direct diagnostic platform instead of simply being a biomarker discovery platform. Other platforms for highly parallel measurements of gene expression include SAGE and MPSS (56), but these methods are technically challenging. MPSS can provide the exact number of an RNA molecule per cell, even the ones at very low levels. Thus, MPSS might be used to confirm results from microarrays.

Definition of Subsequences Within “Genes”

The first step in the reduction to an alternative platform involves a statistical reduction of the number of specific transcriptional markers that are required to still make a high percentage of classifications with an acceptable probability of error. Unlike discoveries of “gene expression” using microarrays prepared using cDNA molecules (several hundred base pairs of double stranded DNA) or even long oligonucleotides (e.g. single-stranded 70-mers), the Affymetrix gene expression microarrays probe all known genes with a combination of at least ten 25-mer probe pairs across the wherein one of the pair members is a perfect sequence match to the predicted gene sequence and the other is a mismatch, comprised of the same sequence as the its partner except for the middle (number 13 position) nucleotide. Complementary binding between a 25-mer probe and its target transcriptional marker is severely attenuated by even a single mismatch (unlike long oligonucleotide and cDNA probes). Hence, it is critical to recognize that only small oligonucleotide probes provide probe-wise interrogation of the highly heterogeneous transcriptome, the content of which varies with not only gene activation and deactivation but also with alternative exon splice variation, depending on exact physiological conditions.

Although the GCOS software makes “present” or “absent” calls for a known or predicted full length gene sequence based on an algorithm which considers the probe pair intensity profiles across the three prime end of the gene sequences, the result can be de-convoluted into individual probe pair intensities. The intensity values that are available for each probe set within each known gene sequence are relatively high confidence sequence identifications that are independent of whether that 25-mer transcriptional sequence has been spliced into different resultant mRNAs. A cDNA probe for a full length gene product would be entirely incapable of making such a discrimination, and the 70-mer probe array should show intermediate level of sequence determination, but would require higher hybridization stringency. Moreover, the error rate in a transcriptional sequence determined from the long oligonucleotide 70-mer would be intermediate to high inaccuracies.

Reduction of Subsequence Content

In a manner similar to that described in the present invention for reducing the number of full sequence genes required to make classifications, the number of subsequences within the full length gene sequences may also be selected for use in classification, irrespective of whether the Affymetrix GCOS software identified the full length “gene” as being “present” or “absent”. In this manner, the classification problem will be reduced to a set of defined 25-mer subsequences having experimentally-verified abundance variations instead of full-length gene sequences which will be comprised of subsequences might or might not actually be present or change in abundance.

Alternative Assay Design

The Affymetrix GeneChip® platform provides an excellent format for the discovery genome-wide expression changes in research, and possibly for clinical diagnostics in situations that allows one or more days for a result (e.g. tumor prognosis). However, many applications, including infectious diagnostics, will be more critically time-dependent. Ideally, these assays will be performed in several hours.

In several very preferable embodiments, the information gleaned from whole genome GeneChip® experiments will be used produce a greatly reduced set of markers that can be measured rapidly in an alternative format that is optimized for both speed and simplicity. In one very preferable embodiment, a reduced set of gene expression markers is analyzed by reverse transcription PCR (RT/PCR) without requiring isolation of total RNA. An example of this can be found with the Ambion (Austin, TX) “Cells-to-Signal™” Kit, which allows RT/PCR amplification directly from cell lysates following a 5 minute incubation with the reagent, bypassing the need for mRNA isolation. Such a technique might be applied to whole. blood lysates or to lysates of specific cell types that are separated from whole blood by any of a number of methods, including centrifugation, fluorescence-activated cell sorting (FACS), or by other flow cytometry techniques, such as with the use of the Agilent Bioanalyzer 2100 or the like.

The cDNA products from the preparations described above can be analyzed directly in small numbers using real-time PCR techniques (e.g. TaqMan, or Fluorescence Energy Transfer (FRET) techniques, molecular beacons, etc.) or in larger numbers using DNA microarrays having a much smaller probe content than the whole genome Affymetrix GeneChips in a system that is optimized for speed and simplicity (57). The microarrays used for this purpose could be selected from a large number of options described in a previous overview (58).

In a highly preferred embodiment, the volume of blood required to perform an assay of the type described above would be greatly reduced relative to that required for the experiments described in the present invention.

There are two small aliquot techniques available on the market currently. Both can amplify from nanograms amount of RNA to microgram amounts. One is from Affymetrix which supports its two-cycle amplification protocol. This protocol basically doubles the in vitro transcription step to obtain more cRNA products. Of course, this would also increase the workload and the time considerably. A new protocol for amplifying nanograms of RNA in a relative short time is available from Ovation™. Although this technique has not been extensively tested on the Affymetrix system, it holds much promise and is contemplated by the present invention. By these techniques only a few drops of blood is needed to isolate nanograms of RNA. Additional methods may be developed to collect drops of blood and RNA stabilization. One such possibility is to use RNAstat to stabilize the blood and for transportation and storage, followed by RNA isolation when needed.

Alternatively, the information obtained from whole genome GeneChip® experiments could be used produce assays that probe for the polypeptides that are coded for by the transcriptional markers detected by the GeneChip® whole genome assay. These polypeptides could be detected in blood or from cell lysates using microarrays comprised of antibodies (59) instead of DNA probes or by mass spectrometry methods that measure relative protein abundances.

As Part of an Overall Business Model

However, it is a central hypothesis of the Epidemic Outbreak Surveillance (EOS) program and the present invention that the only economical method to realistically widely deploy a parallel pathogen surveillance assay in a clinical environment is to do so in parallel with assays that have validity in their own right for routine clinical diagnosis of common pathogens. That is, unlike a reimbursable diagnostic assay for a common pathogen, an un-reimbursable assay for bioweapons surveillance will only burden a clinical operation and will not be widely adopted. Because it may not always be possible to identify the specific cause of an infection through pathogen genomic markers (e.g. using PCR or microarrays), there remains a critical need to determine alternative “biomarkers' from the host that would elucidate the character of the disease etiology and guide the clinician in the proper management of the infection. Gene expression monitoring is thought of as a potentially revolutionary technology that could provide hundreds if not thousands of such “biomarkers”. However, in order for gene expression-based bio-defense assays to move beyond scientific curiosity and into the realm of clinical diagnostics, a significant work must be carried out to demonstrate that the principle is applicable to routine clinical diagnostics. Hence, there is a critical need to develop databases of baseline (normal) human gene expression levels and to understand the nature of perturbations caused by various levels and stages of pathogen infection.

The above written description of the invention provides a manner and process of making and using it such that any person skilled in this art is enabled to make and use the same, this enablement being provided in particular for the subject matter of the appended claims.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Overview

Informed consented Basic Military Trainees (BMTs) generously donated blood and/or nasal washes. Blood collection and RNA isolation was performed using the Paxgene Blood RNA System (PreAnalytiX), which consists of an evacuated tube (PAX tube) for blood collection and a processing kit (PAX kit) for isolation of total RNA from whole blood (35). The isolated RNA was amplified, labeled, and interrogated on HG-U133A (A) and HG-U133B (B) Genechips from Affymetrix. The Affymetrix GeneChip platform measures a significant subset of the transcriptome. In design, it incorporates a DNA oligonucleotide microarray, manufactured via photolithography to detect labeled cRNA targets amplified from RNA populations. Nasal washes were aliquot and sent for determination of adenovirus infection via culture and real-time PCR.

### Example 1

### Sample Collection

Lackland Air Force Base (LAFB) in San Antonio, Tex. is the location of Basic Military Training for all recruits to the United States Air Force. More than 50,000 Basic Military Trainees (BMTs) undergo a 6 week training course prior to assignment of duty. These BMTs are organized into flights of 50-60 individuals that eat, sleep and train in close quarters. Each flight is paired with a brother or sister flight with which there is increased contact due to co-localization for scheduled activities and multiple flights are grouped into squadrons which reside in the same dormitory building, subdivided into dorms for individual flights.

BMTs arriving to LAFB underwent informed consent to participate in this study. On day 1-3 of training, approximately 15 milliliters of blood were drawn from each BMT into a total of 5 Paxgene tubes, per standard protocol, to establish baseline gene expression profiles. BMTs who presented during training with a temperature of 100.5 or greater and respiratory symptoms were consented for a nasal wash and Paxgene blood draw. All Paxgene tubes were maintained at room temperature for 2 hours and then were frozen at −20 C and shipped on dry ice to the Naval Research Laboratory (NRL) within 7 days for processing. Nasal washes were performed by standard protocol using 5 cc of normal saline to lavage the nasopharynx with collection of the eluent in a sterile container. Nasal wash eluent was stored at 4° C. for 1-24 hours before being aliquoted and stored at −20° C. and shipped to NRL within 7 days for processing.

All BMTs underwent a standardized questionnaire at initial presentation, during presentation with illness, and at follow-up. Questions posed to BMTs include: vaccination history, allergies, last meal, last exercise, last injury, medication taken, smoking history, observed subjective symptoms, and last menstruation (if appropriate). Among the observed subjective symptoms asked and monitored are: sore throat, sinus congestion, cough (productive or non-productive), fever, chills, nausea, vomiting, diarrhea, malaise, body aches, runny nose, headache, pain w/deep breath, and rash. All data was stored in electronic format using personal identification numbers.

The present inventors sought to determine the gene expression patterns that developed in Basic Military Trainees (BMT) populations as they were naturally exposed to respiratory pathogens and subsequently developed disease during their 6 week training period. Up to 50% of BMTs experience upper respiratory tract infection (URI) during training and 40% of these will have fever and URI symptoms. Approximately 60-80% of febrile respiratory disease is due to adenovirus type 4. Other pathogens that cause a significant minority of disease include Streptococcus pyogenes, Chlamydia pneumoniae, Mycoplasma pneumoniae, and Bordetella pertussis.

BMTs maintain set schedules throughout the 6 week training program and are kept in close proximity; the BMT population offers a unique opportunity to evaluate gene expression profiles resulting from pathogen exposure and/or infection in the absence of confounding external/environmental factors.

In the first 18 months of the EOS program, a Lackland and Air Force Surgeon General Institutional Review Board (IRB)-approved protocol was implemented. This protocol continues to be supported by the Lackland 37^{th} Training Wing Commander and the Base Commander. The present inventors implemented an experimental model for comparing whole blood expression profiles from four categories of BMTs:

1. Healthy (baseline),

2. Febrile Respiratory Illness (FRI) adenovirus 4 infected (Ad4+),

3. FRI without adenovirus (Ad4−), and

4. post-FRI Ad4+(individuals recovered from adenoviral infection, i.e. #2 above).

Individuals were identified as healthy if they were in week 0 of basic training and had no respiratory symptoms in the prior 4 weeks. Individuals with FRI were identified by primary providers and study nurses as the BMTs presented to health clinics and dispensaries. All BMTs were consented and underwent blood draw to determine gene expression profiles. All ill BMTs were administered a standardized questionnaire to determine the type of presenting symptoms and the onset and duration of symptoms. Physical examination and complete blood counts were recorded. BMTs who were determined to have an adenoviral illness by rapid immunoassay/PCR/culture underwent a subsequent blood draw and nasal wash 14-21 days after their initial FRI presentation; the majority of these individuals had no further symptoms of infection at the time of the follow-up blood draw. PCR for adenovirus and culture for all respiratory viruses was performed on nasal washes. One hundred BMTs were entered on the study, including 30 healthy BMTs. Whole blood gene expression profiling for 33,000 known genes and open reading frames (ORFs) was performed on PAXgene blood RNA samples using Affymetrix U133A/B chip sets. Data from 76 BMTs is available with the following breakdown: healthy (n=38), febrile without adenovirus infection (n=14), febrile with adenovirus infection as determined by culture (n=24), and those who recovered from adenovirus associated febrile illness (n=26). Initial search for genes that show expression level differences of >=1.5 fold-change of the lower 90% confidence interval between groups showed that: 913 genes differ between healthy and febriles at 0.1% median false discovery rate (FDR); 203 genes differ between healthy and recovered at 2.0% FDR. Ongoing recruitment with the addition of a screening rapid assay for adenovirus has enabled increased enrollment of FRI Ad4− BMTs and will enable statistical analysis between the FRI Ad4+ and Ad4− groups.

### Example 2

### Sample Preparation

Materials and Methods

PAX tube blood collection. Blood was collected into the PAX tubes from volunteers according to the manufacturer's directions (60). For the experiment described in FIG. 1, twelve PAX tubes were collected from one person. Then, the tubes were split into two groups of six for the two conditions. Subsequently, RNA from pairs of tubes had to be pooled to obtain enough RNA for further processing. This resulted in three replicates in each condition.

Total RNA isolation. After sample collection, the PAX tubes were incubated at room temperature for 2 or 9 hours, followed by immediate total RNA isolation or freezing at −20° C. for 6 days before further processing. For total RNA isolation, we followed the PAX kit handbook (33), but with modifications to aid tight pellet formation after proteinase K treatment. Loose pellets were problematic. To form tight pellets, we increased the proteinase K added from 40 μl to 80 μl (>600 mAU/ml) per sample and the 55° C. incubation time from 10 min to 30 min. After spinning the samples, if a tight pellet still did not form, then we remixed the samples, incubated at 55° C. for another 5 min, and followed by centrifugation. The optional on-column DNase digestion mentioned in the PAX kit handbook was not carried out. Thus, OD measurements at this point would not give accurate quantification due to DNA contamination; however, the 260/280 ratio may indicate other contaminants. Approximately 4 82 l of the 80 μl eluted RNA was needed to obtain an absorbance greater than 0.1. All aliquots were diluted in 10 mM Tris-Cl pH 7.5 for OD readings.

In-solution DNase digestion. Subsequently, in-solution DNase treatment was carried out using the DNA-free™ kit (Ambion). Briefly, for each sample eluted in 80 μl BR5 buffer, we added 7 μl 10× DNase I buffer and 1 μl DNase, followed by mixing and incubation at 37° C. for 20 min. Afterwards, 7 μl of DNase inactivation reagent was added, incubated at room temperature for 2 min, and spun down to pellet the beads that were in the inactivation reagent. The treated RNA in the supernatant was pipetted off without disruption of the pellet. An aliquot of each RNA sample was run on the bioanalyzer for quantification and QC measurements.

Poly-A RNA isolation. After DNase treatment, duplicate samples were pooled, and mRNA was isolated using the Oligotex™ mRNA kit (Qiagen). The mRNA was eluted in 100 μl total of OEB buffer.

Sample concentration. Next, the samples were concentrated via ethanol precipitation. For each 100 μl sample, we added 1 μl glycogen (5 mg/ml) (Ambion), 15 μl 5M ammonium acetate, and 200 μl 100% ethanol chilled at −20° C. The reaction was incubated at −20° C. overnight. The next day, the samples were spun down at 13,791 g at 4° C. for 30 min. The pellet was washed twice with 80% ethanol chilled at −20° C.; air-dried; and resuspended in 12 μl of nuclease free water (Ambion).

Generation of cRNA. All subsequent steps were carried out as described in the GeneChip® expression analysis manual (6). Ten microliters of each sample were used in the first strand cDNA synthesis reaction. Ten microliters of purified double-stranded cDNA were used for synthesis of biotin-labeled cRNA. Fragmentation, hybridization, and detection were performed as described in the manual (6).

Measurements on the bioanalyzer. One microliter, from pre- and post-DNase total RNA, purified double stranded cDNA, purified cRNA diluted 1:10, and fragmented cRNA, was run on the bioanalyzer using the protocols described in the RNA 6000 Nano Assay (Agilent Technologies) (61). The usage of the bioanalyzer was analogous to gel electrophoresis, except that the gel matrix and samples were flowed through microfluidic channels of a cartridge, thus facilitating small sample usage and automated quantification.

Real-time PCR for gapdh gene. Each real-time PCR reaction for gapdh DNA included: 12.5 μl 2× SYBR green PCR master mix (Applied Biosystem), 0.5 μl 5′GTGAAGGTCGGAGTCAACGG forward primer (10 μM), 0.5 μl of 5′GCCAGTGGACTCCACGACGTA reverse primer (10 μM), 10.5 μl of water, and 1 μl of template from total RNA or cDNA samples. The reactions were carried out in the iCycler (Biorad) with cycling settings of 95° C. 3 min; 95° C. 30 s, 58° C. 30 s, and 72° C. 30 s for 40 cycles; followed by melting curve analysis and/or a 4° C. hold. The completed reactions were also analyzed by gel electrophoresis.

Reverse transcription. For RNA quality assessment during protocol development, synthesis of cDNA was carried out using the SuperScript™ First-Strand synthesis system for RT-PCR kit (Invitrogen Life Technologies).

Statistical analysis. Statview (SAS Institute) software was used to perform the nonparametric Mann-Whitney U test to determine statistically significant differences between 260/280 OD ratios, concentrations via 260 nm absorbance, concentrations via integration of fluorescence profiles, relative amounts of contaminating DNA via threshold cycle, RNA quality via ribosomal 28S/18S peak ratios, double stranded cDNA yields, purified cRNA yields, and 260/280 ratios of purified cRNA. A P-value of less than or equal to 0.05 was considered statistically significant.

Affymetrix Microarray Suite 5.0 (MAS 5.0) (62) was used for generation of QC metrics including: noise(RawQ), an indicator of variation in pixel intensities; average background; scale factor, an indicator of variation of intensities between chips; percent present calls, an indicator of the number of genes detected; and gapdh 3′/5′ signals and actin 3′/5′ signals, indicators of RNA degradation. Dataplot (63) was used to assess autocorrelations of QC metrics. Statview was used to make individual line charts and to set quality control limits at ±3 standard deviations from the mean.

MAS 5.0 CEL files, which contained intensity values of each probe, and gene expression present calls were imported into dChip (64, 65) for further analysis. In dChip, HG-U133A and HG-U133B chips were analyzed separately. dChip uses intensity values of probes on multiple arrays to calculate an expression index, which is a measure of transcript abundance. The expression index is analogous to the signal statistic output by MAS 5.0. dChip was used for hierarchical clustering and fold-change determinations, and the expression indices were exported to JMP IN (SAS Institute) for analysis of variance.

Results

Adaptation of RNA from PAX tube for use with the GeneChip® system. RNA from a PAX tube was isolated using the protocol provided with the PAX kit. As determined by spectrometry, the yield was 4.8 μg; the 260/280 ratio was 2.01; and the concentration was 0.06 μg/μl. This was not sufficient for use with the GeneChip® protocol which prescribed an initial total RNA amount of 5 μg at 0.5 μg/μl (6). Thus, RNA isolated from two PAX tubes were pooled, followed by ethanol precipitation and resuspension in 15 μl of BR5 buffer. This resulted in a yield of 10.4 μg, a 260/280 ratio of 2.07, and a concentration of 0.7 μg/μl, which met the amounts recommended in the GeneChip® protocol.

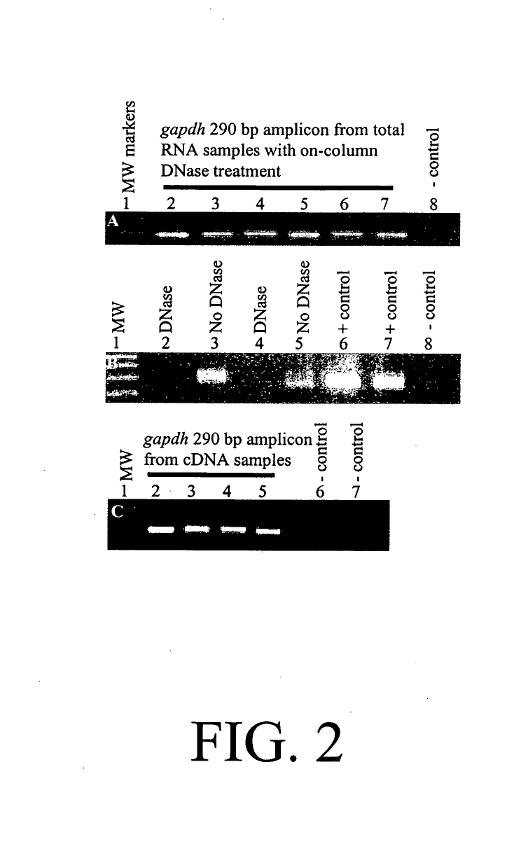

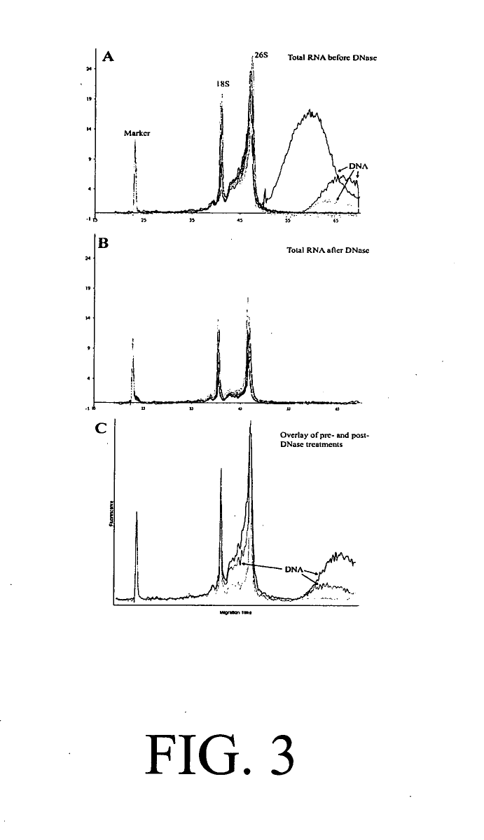

The optional on-column DNase digestion step was performed as described in the PAX kit. However, for quality assurance, the presence of DNA in the purified RNA was assessed via real-time PCR for the gapdh gene. PCR could detect the presence of gapdh DNA (FIG. 2A), suggesting that the on-column DNase digestion was not efficient enough to remove DNA to a level undetectable by PCR. Thus, the RNA was treated with DNase in solution. Afterwards, gapdh DNA was not detected by real-time PCR (FIG. 2B), suggesting that most DNA had been digested. However, the RNA integrity may be compromised during in-solution DNase treatment; thus, reverse transcription followed by real-time PCR for gapdh was performed on the in-solution DNase treated samples. The gapdh DNA was detected following reverse transcribed-PCR (FIG. 2C), suggesting that the RNA was still of good quality.

The use of Oligotex purified mRNA was based on a preliminary experiment comparing the number of genes detected when using total RNA versus mRNA isolated from blood in PAX tubes. The resulting present calls, signifying the number of genes detected, were 33% for total RNA and 41% for mRNA on the HG-U133A chips. Comparisons were also made between mRNA isolated via Oligotex and mRNA isolated via ion-pair reversed-phase high performance liquid chromatography (IP RP HPLC) (66). The resulting present calls were 17% and 19% for IP RP HPLC and 35% and 40% for Oligotex mRNA. Since Oligotex isolated mRNA showed the highest percent present calls, the step was incorporated into the protocol.

The protocol used for gene-expression profiles of human blood samples using the PAXgene Blood RNA System and the GeneChip® platform includes at least 2 PAX tubes per donor, total RNA isolation without on-column DNase digestion but with in-solution DNase digestion, mRNA isolation, precipitation for concentration, followed by standard protocols from the GeneChip® manual.

Comparison of QC measures for conditions E and O. We compared the quality control measures of PAX tube-collected blood samples whose RNA were isolated after the minimum incubation time of 2 hours at room temperature (FIG. 1, condition E) and after incubation at room temperature for nine hours followed by storage at −20 C for 6 days (FIG. 1, condition O).

To compare the purity and yield of total RNA from the two conditions, we performed spectrometric analysis on the RNA samples. There was no difference in the 260/280 ratio between the two treatments (Table 1, row 1), suggesting that RNA purity was equivalent for the samples. The yield before DNase treatment was 1.0 μg higher for condition E than O (Table 1, row 2). However, this measure may be confounded by differential DNA contamination in the samples. Thus, after in-solution DNase treatment, we quantitated the RNA using the bioanalyzer (FIG. 3B). Surprisingly, the yield was 0.9 μg higher in condition O than E (Table 1, row 3). This implied that there was more DNA contamination in E compared to O. Therefore, we measured the relative amount of DNA contamination in the two treatments via real-time PCR for gapdh. The threshold crossing cycle was lower in E compared to O (Table 1, row 4), indicating that there was more DNA in E. These observations indicated that more DNA contamination occurred in E-than O but that the yield of RNA was higher in O than E.

TABLE 1
Comparisons between condition E versus O of quality metrics relating purity,
yield, and stability of total RNA isolated from PAX tube. Each mean ± SEM value displayed
in each cell was calculated from n = 6.

| | | | | | | Mann- |
|---|---|---|---|---|---|---|
| | | Treatment | | | | WhitneyU |
| | | of RNA | | Condition E | Condition O | test |
| Row # | Description | samples | Method | (mean ± SEM) | (mean ± SEM) | P-value |
| 1 | Purity via 260/280 | No DNase | Spectrometry | 2.07 ± 0.04 | 2.07 ± 0.05 | 0.631 |
| | OD ratio | | | | | |
| 2 | Concentration via | No DNase | Spectrometry | 7.3 ± 0.2 μg | 6.3 ± 0.2 μg | 0.007* |
| | 260 Absorbance | | | | | |
| 3 | Concentration via | In-solution | Bioanalyzer | 3.8 ± 0.2 μg | 4.7 ± 0.2 μg | 0.025* |
| | integration of | DNase | | | | |
| | fluorescence | | | | | |
| | profiles | | | | | |
| 4 | Relative amounts | No DNase | Realtime PCR | 14.7 ± 0.8 | 24.3 ± 0.6 | 0.004* |
| | via threshold cycle | | for gapdh DNA | | | |
| 5 | RNA quality via | In-solution | Bioanalyzer | 1.7 ± 0.1 | 1.6 ± 0.1 | 0.200 |
| | 28S/18S peak ratio | DNase | | | | |

RNA from various samples produced different profiles on the bioanalyzer, and we would like to use such profiles for QC. Therefore, we overlaid RNA profiles from our samples to assess inter-sample variability and RNA quality (FIG. 3). Before DNase treatment, fluorescence profiles from condition E were, on average, higher than samples from O (FIG. 3A). After in-solution DNase treatment, the fluorescence profiles decreased overall and reversed with respect to the conditions (FIG. 3B). Interestingly, comparisons of pre- and post-DNase treatment profiles suggested that DNA tended to show up between the two ribosomal peaks and as a hump at later times (FIG. 3A & C). These observations corroborated the yield and DNA contamination results determined by spectrometry and real-time PCR. The ratios of the 28S to the 16S ribosomal RNA peaks averaged around 1.6 (Table 1, row 5) based on the bioanalyzer automatic peak detection and calculation software. However, manual adjustment indicated that the 28S/16S ratio averaged around 2. There was no difference in the 28S/16S ratio between condition E and O (Table 1, row 5). The shapes of the fluorescence profiles were similar in both treatments (FIG. 3B). These results suggested that the RNA populations from both conditions were of similar good quality.

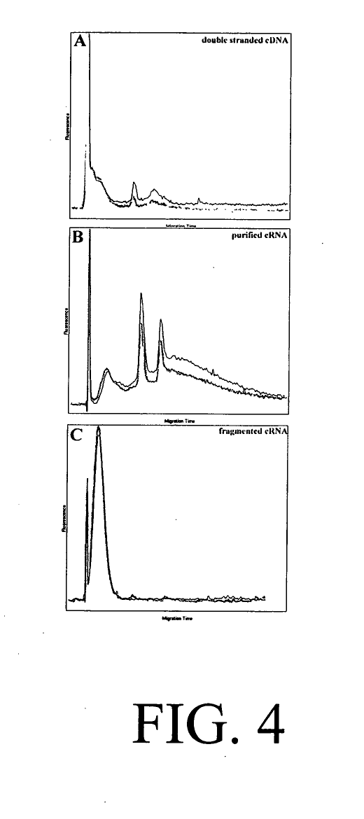

Since the RNA were of similar quality for the two conditions, we continued through the procedures to make fragmented labeled cRNA. We used the bioanalyzer to monitor double stranded cDNA synthesis (FIG. 4A), purified cRNA (FIG. 4B), and fragmented cRNA (FIG. 4C). The characteristic profiles in FIG. 4 were indicative of successful reactions. The yield of double stranded cDNA was 0.09 μg higher in condition E than O (Table 2, row 1), while the yield of purified cRNA was around 30 μg with no detectable differences between the two conditions (Table 2, row 2). The 260/280 ratios were similar between the two groups (Table 2, row 3).

TABLE 2
Comparisons between condition E versus O of quality metrics relating yields and
purity of double stranded cDNA and cRNA derived from mRNA isolated from PAX tube.
Each mean ± SEM value displayed in each cell was calculated from n = 3.

| | | | Condition E | Condition O | Mann-Whitney U |
|---|---|---|---|---|---|
| Row # | Description | Method | (mean ± SEM) | (mean ± SEM) | test P-value |
| 1 | Double stranded | Bioanalyzer | 0.56 ± 0.03 μg | 0.47 ± 0.03 μg | 0.050* |
| | cDNA yield | | | | |
| 2 | Purified cRNA yield | Spectrometry | 34 ± 4 μg | 30 ± 3 μg | 0.513 |
| 3 | 260/280 of purified | Spectrometry | 2.3 ± 0.03 | 2.4 ± 0.06 | 0.275 |
| | cRNA | | | | |

Since the QC metrics suggested that sample preparation was successful, we hybridized the samples to human HG-U133A chips followed by hybridization onto the HG-U133B chips using the same hybridization cocktails, which had been stored at −80° C. Hybridization, washing, detection, and scanning were done as described in the GeneChip® manual (6).

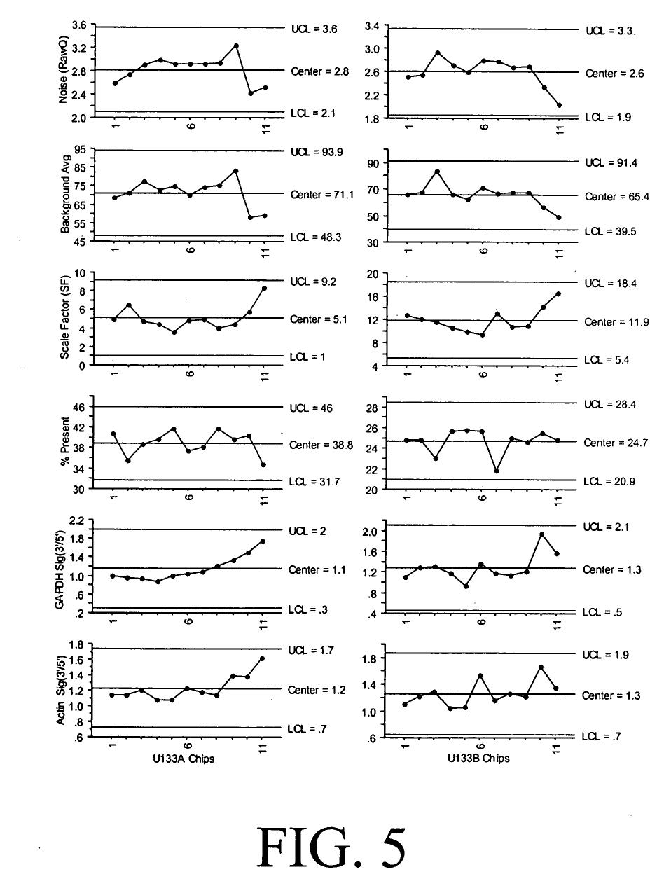

Afterwards, we assessed the QC metrics along with other samples processed in our facility (FIG. 5). To determine if the metrics were fluctuating randomly over time, each QC metric shown in FIG. 5 was graphed on lag- and autocorrelation plots (not shown) (67). There was no obvious pattern in the plots, suggesting that the metrics were randomly drawn from a fixed distribution, thus enabling the setting of control limits at ±3 standard deviations from the center mean. All measures were within the control limits. Average Background centered around 70, which was within the typical range of 20 to 100 (68). Importantly, the percent present centered at 39% for HG-U133A chips and 25% for HG-U133B chips. Finally, the 3′ to 5′ signal ratio for both gapdh and actin centered at ˜1.2, indicating that the RNA was of good quality and cRNA synthesis was efficient. Comparisons of these QC metrics for the samples from conditions E and O indicated no significant differences. These QC results suggested strong confidence in the reliability of our process.

Analysis of gene-expression profiles. To determine the contributions of handling conditions, microarray chips, and differing genes to the variation in measures of transcript abundance, we performed a three-way analysis of variance on dChip-derived gene expression indices from HG-U133A chips. Quantile-normal plot of expression indices from 6 chips indicated that the expression indices were not normally distributed. Thus, 100 genes were randomly sampled from the 22,577 genes, and their expression indices were transformed by adding ‘1’ to every value to remove zeros followed by a Box-Cox transformation to bring the distribution closer to normality. Subsequently, the transformed data was fitted into the following model:
Yᵢⱼₖ=M+Cᵢ+Pⱼ+Gₖ+Eᵢⱼₖ

Where Y stands for the transformed expression indices, M for the grand mean, C for the two conditions (i=1, 2), G for the 100 sampled genes (k=1, 2, 3, . . . 100), and E for the residual error. P has three levels (j=1, 2, 3) and encompasses variations due to the order of the blood draw, order of processing, and/or between chips. For example, level j=1 of P contains expression indices from one chip of each condition, and these two chips detected targets from PAX tube samples that were drawn first (draw order numbered 1, 3 for condition E and 2, 4 for condition O, FIG. 1) and processed together. After model fitting, the residual versus predicted plot showed no correlation, and the residuals were normally distributed (Shapiro-Wilk W test, P=0.24). The coefficient of determination (R²) was 0.993. These results suggested that the model adequately explained most of the variation in the data. The analysis of variance results are shown in Table 3.

TABLE 3
3-Way ANOVA results

| | Degree of | Sum of | % of total | | | |
|---|---|---|---|---|---|---|
| Source | freedom | Squares | variation | Mean Square | F ratio | P-value |
| Condition (C) | 1 | 50,843 | 0.090 | 50,843 | 60.2 | <0.0001 |
| Chip (P) | 2 | 94,662 | 0.167 | 47,331 | 56.1 | <0.0001 |
| Gene (G) | 99 | 56,189,455 | 99.004 | 567,570 | 672.4 | 0.0000 |
| Residual (E) | 497 | 419,519 | 0.739 | 844 | | |

The ‘Sum of Squares’ column indicates the magnitude of the variations explained by the factors listed under the ‘Source’ column, while the ‘% of total variation’ column converted the sum of squares into percentages. The F ratio (mean square of a factor/mean square of the residual) is used to test whether the variation explained by a factor is statistically greater than the variation of the residuals; a P-value of less than 0.05 indicated statistical significance. The results indicated that all three factors: C, P, and G, significantly explained portions of the total variation. However, the gene (G) factor explained most of the variation (99%), while the handling conditions contributed minimally (0.09%) to differences in gene expression levels. These results were generalizable to all genes on the chips since the 100 genes analyzed were randomly selected.

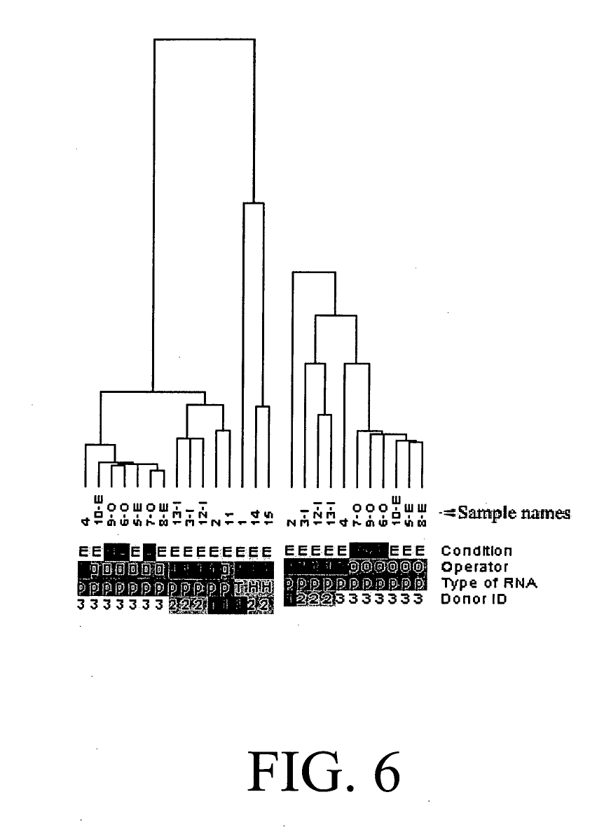

To determine the correlations of gene levels among the samples of the two conditions relative to other PAX-tube-derived samples processed in our lab, cluster analysis was performed. Samples were clustered via hierarchical clustering with average linkage, no gene filtering, and no standardization of genes or samples. The distances among samples were l-r, where r is Pearson's linear correlation coefficient. This distance measure quantified dissimilarities between entire expression profiles. The resulting dendrograms with descriptive ontologies of samples are shown in FIG. 6. The samples from conditions E and O clustered together away from samples that differed by other factors such operator and individual donors, and they segregated into E and O conditions for genes on the HG-U133B chips. This result further support the analysis of variance in that the differing conditions did not induced large changes in gene profiles.

To quantitate differences between the two conditions in terms of fold-changes, we compared fold changes of all genes between the conditions. From the set of non-filtered genes (˜22,600 genes for HG-U133 chips, with 7,600 genes for HG-U133A and 5600 genes for HG-U133B called present by MAS 5.0), we filtered for genes that showed greater than 1.3 fold changes between the conditions using the lower bound of the 90% confidence interval of fold-change estimates. This resulted in 5 genes for HG-U133A chips and 22 genes for HG-U133B chips (Table 4). When the lower bound was set to 1.5, only 1 gene remained for HG-U133A chips and none for HG-U133B chips. These results indicated that the differences between the two conditions were due to genes whose expression indices differ by no more than 1.5 fold of the 90% lower bound.

TABLE 4
List of genes that showed greater than 1.3 fold change using the lower bound of
the 90% confidence interval between condition E and O

| | | | | | | |
|---|---|---|---|---|---|---|
| | | | | | Lower bound | Upper bound |
| | | E | O | Fold | of fold- | of fold- |
| probe set | gene | mean¹ | mean² | change | change | change |

U133A chips

| | | | | | | |
|---|---|---|---|---|---|---|
| 200032_s_at | ribosomal protein L9 | 731.73 | 1272.5 | 1.74 | 1.31 | 2.18 |
| 204661_at | CDW52 antigen (CAMPATH-1 antigen) | 834.26 | 1394.3 | 1.67 | 1.34 | 2.02 |
| 206207_at | Charot-Leyden crystal protein | 657.73 | 1085.4 | 1.65 | 1.36 | 1.96 |
| 210510_s_at | neuropilin 1 | 224.6 | 492.39 | 2.19 | 1.9 | 2.54 |
| 211264_at | glutamate decarboxylase 2 (pancreatic islets | 30.97 | 49.3 | 1.59 | 1.3 | 2 |
| | and brain, 65 kD) | | | | | |

U133B chips

| | | | | | | |
|---|---|---|---|---|---|---|
| 222787_s_at | hypothetical protein FLJ11273 | 168.39 | 106.06 | −1.59 | −1.41 | −1.79 |
| 222791_at | hypothetical protein FLJ11220 | 226.09 | 142.84 | −1.58 | −1.39 | −1.84 |
| 222793_at | RNA helicase | 754.62 | 490 | −1.54 | −1.36 | −1.73 |
| 222833_at | hypothetical protein FLJ20481 | 317.62 | 221.84 | −1.43 | −1.32 | −1.56 |
| 223243_s_at | chromosome 1 open reading frame 22 | 206.55 | 135.11 | −1.53 | −1.33 | −1.78 |
| 224737_x_at | Consensus includes gb: BG541830 | 65.17 | 36.26 | −1.8 | −1.47 | −2.23 |
| | /FEA = EST | | | | | |
| 225626_at | phosphoprotein associated with | 307.44 | 205.34 | −1.5 | −1.36 | −1.66 |
| | glycosphingolipid-enriched | | | | | |
| 226119_at | similar to hypothetical protein FLJ10883 | 299.72 | 185.48 | −1.62 | −1.39 | −1.89 |
| 226148_at | Consensus includes gb: AU144305 | 274.02 | 183.58 | −1.49 | −1.35 | −1.66 |
| | /FEA = EST | | | | | |
| 226465_s_at | SON DNA binding protein | 243.4 | 154.52 | −1.58 | −1.4 | −1.77 |
| 226641_at | Consensus includes gb: AU157224 | 715.14 | 457.8 | −1.56 | −1.34 | −1.86 |
| | /FEA = EST | | | | | |
| 226979_at | mitogen-activated protein kinase kinase | 408.84 | 261.97 | −1.56 | −1.35 | −1.82 |
| | kinase 2 | | | | | |
| 227405_s_at | frizzled homolog 8 (Drosophila) | 636 | 373.97 | −1.7 | −1.41 | −2.01 |
| 227772_at | Consensus includes gb: AV700849 | 211.74 | 138.2 | −1.53 | −1.32 | −1.8 |
| | /FEA = EST | | | | | |
| 228248_at | Consensus includes gb: W49629 /FEA = EST | 549.67 | 356.03 | −1.54 | −1.31 | −1.83 |
| 228328_at | Consensus includes gb: AI982758 /FEA = EST | 158.3 | 102.72 | −1.54 | −1.32 | −1.82 |
| 232744_x_at | Consensus includes gb: BG485129 | 27.38 | 16.57 | −1.65 | −1.41 | −1.96 |
| | /FEA = EST | | | | | |
| 237403_at | Consensus includes gb: AI097490 /FEA = EST | 979.37 | 603.12 | −1.62 | −1.37 | −1.95 |
| 240784_at | Consensus includes gb: BE549627 | 624.51 | 390.38 | −1.6 | −1.38 | −1.85 |
| | /FEA = EST | | | | | |
| 241202_at | Consensus includes gb: AA779283 | 676.47 | 416.03 | −1.63 | −1.31 | −2.01 |
| | /FEA = EST | | | | | |
| 241260_at | Consensus includes gb: N39326 /FEA = EST | 13.67 | 22.95 | 1.68 | 1.39 | 2.04 |
| 243589_at | Consensus includes gb: AI823453 /FEA = EST | 264.88 | 160.86 | −1.65 | −1.41 | −1.91 |

¹The mean of expression indices of condition E (n = 3)
²The mean of expression indices of condition O (n = 3)

In comparing the two conditions, there were more genes that showed changes on the HG-U133B chips than on the HG-U133A chips, even though more genes were detected on the HG-U133A chips. Also, the genes that changed on the HG-U133B chips mostly went down in condition O.

Our results implied several recommendations as to sample handling for multi-centered studies. Since there were differences between the conditions but they both showed good within-group reliability, one should preferably pick one method to reduce variability. In which case, condition O seemed advantageous over E, as it provided time before one had to process or freeze the samples and allowed for transportation while frozen. If one needed the flexibility of the range of handling methods between the conditions, then this would still be possible, as long as during subsequent analysis, one increased statistical stringency, such as only passing genes greater than 1.5 fold change of the 90% lower bound.

### Example 3

### GXP Program “Quad30” Experiments

Materials and Methods

Culture of adenovirus from nasal washes. All samples are cultured for Adenovirus, Parainfluenza 1, 2, and 3, Influenza A and B and RSV. Standard cell types, including Rhesus Monkey Kidney-PMK or Cynomologous Monkey Kidney-CYN are most commonly used in addition to A549 cells. Standard culture and shell vial with direct fluorescent antibody are used. All respiratory cultures are held for 10-14 days until called negative.

Fluorogenic real-time PCR for adenovirus serotype 4 from nasal washes. DNA was extracted from 100 μl of nasal washes using the MasterPure™ DNA purification kit (Epicentre Technologies, Madison, Wis.) and resuspended in 10 μl nuclease free water (Ambion Inc., Austin, Tex.). Two different fluorogenic real-time PCR were used to detect adenovirus serotype 4 hexon and fiber genes. For hexon gene specific PCR, each reaction was 15 μl total volume containing 20 mM Tris-HCl (pH 8.4), 50 mM KCl, 4 mM MgCl₂, 200 μM dNTPs (Invitrogen Life Technologies, Carlsbad, Calif.), 200 nM primers, 100 μM TaqMan probe (Integrated DNA technologies, Inc. Coralville, Iowa), 0.6 U of Platinum Taq DNA polymerase (Invitrogen Life Technologies, Carlsbad, Calif.), and 0.6 μl purified DNA from nasal washes. The sequences of adenovirus 4 specific hexon primers are: 5′-GTTGCTAACTACGATCCAGATATTG-3′ (forward; SEQ ID NO:1) and 5′-CCTGGTAAGTGTCTGTCAATCC-3′ (reverse; SEQ ID NO:2). The sequence of adenovirus 4 hexon specific probe is 5′-FAM-CAGTATGTGGAATCAGGCGGTGGACAGC-TAMRA-3′ (SEQ ID NO:3), where FAM is the fluorescent reporter, and TAMRA is the fluorescence quencher. The reaction conditions were: 94° C. 3 min denaturation, then 35 two-step cycles of ramping to 95° C. and 60° C. 20 s. For fiber gene specific PCR, each reaction was also 15 μl total volumes containing 1.5 μl FastStart DNA Master SYBR Green I (Roche Applied Science, Indianapolis, Ind.), 3 mM MgCl₂, 200 nM primers, and 0.6 μl purified DNA from nasal washes. The sequences of adenovirus 4 specific fiber primers are: 5′-TCCCTACGATGCAGACAACG-3′ (forward; SEQ ID NO:4) and 5′-AGTGCCATCTATGCTATCTCC-3′ (reverse; SEQ ID NO:5). The reaction conditions were 94° C. 10 min denaturation, then 40 two-step cycles of ramping to 95° C. and 60° C. 20 s. Both reactions were carried out in the RAPID LightCycle™ (Idaho Technology Inc., Salt Lake City, Utah).

Total RNA isolation from blood. Frozen PAX tubes were thawed at room temperature for 2 hrs followed by total RNA isolation as described in the PAX kit handbook (60), but modified to aid in tight pellet formation by increasing proteinase K from 40 μl to 80 μl (>600 mAU/ml) per sample, extending the 55° C. incubation time from 10 min to 30 min, and the centrifugation time to 30 min or more. The optional on-column DNase digestion was not carried out. Purified total RNA was stored at −80° C.

Target preparation. For more complete removal of DNA from purified RNA samples, RNA isolated from multiple PAX tubes of blood from the same donor at a specific collection date were pulled, followed by in-solution DNase treatment using the DNA-free™ kit (Ambion). However, to facilitate removal of the DNase inactivating beads, the completed reaction was spun through a spin column (Qiagen, Cat#79523), rather than attempting to pipette off the supernatant without disturbing the bead pellet. Subsequently, one micro liter from each post-DNase total RNA sample was run on the bioanalyzer using the RNA 6000 Nano Assay (Agilent Technologies) for assessment of RNA quality and quantification of RNA amount. Next, for most samples, 5 μg of RNA were concentrated via ethanol precipitation. For each 100 μl of RNA sample, we added 1 p72 l glycogen (5 mg/ml) (Ambion), 15 μl 5M ammonium acetate, and 200 μl 100% ethanol chilled at −20° C. The reaction was incubated at −20° C. overnight. The next day, the samples were spun down at 13,791 g at 4° C. for 30 min. The pellet was washed twice with 80% ethanol chilled at −20° C.; air-dried; and resuspended in 10 or 12 μl of nuclease free water (Ambion). All subsequent steps were as described in the GeneChip® Expression Analysis Technical Manual (6).

Database integration. The database can be divided into two major categories: 1) metadata, all information relating to the sample processing that is not gene-expression measurements; and 2) gene-expression data. The metadata consists of several subcategories: clinical, laboratory handling, and quality metrics of microarray results.

Clinical data captures information about the patients as transcribed from the questionnaire, complete blood count (CBC), and about handling of the collected PAX tube blood samples.

Laboratory data contains information about the processing of blood samples. For steps from blood in PAX tubes to total RNA extraction, fields such as date of processing, reagent lots, and operator are captured. Subsequent bioanalyzer measurements of DNased treated RNA samples resulted in fluorescent intensities versus time data, which graphically, form the electropherograms and were treated as metadata as well. The electropherograms were analyzed by the Biosizing (Agilent Technologies) software to output 28S-to-18S intensity ratios and RNA yields, and by the Degradometer 1.1 (51) software to consolidate, scale, and calculate quality metrics such as degradation factors and apoptosis factors. For steps from after bioanalyzer analysis to hybridization, variables such as yields of cRNA and processing batches were recorded.

Quality metrics of microarray results data were information associated with the scanned chip. This included fields such as lot numbers of chips and date of scanned images stored in DAT files. Also included were fields from the Report files generated by the GeneChip Operating Software 1.1 (GCOS 1.1) (Affymetrix), which summarized the quality of target detection for a chip.

Microsoft Access and Excel worksheets were used to enter manually clinical and laboratory handling data. Outputs from Degradometer 1.1 were in Excel worksheets. An in-house script called ReportToMatrix (script provided hereinbelow) was used to reformat and consolidate Report files into a data matrix in Excel. Metadata from GCOS 1.1 were exported into Access.

ReportToMatrix Script:

Sub Macro1( )
filenum=0
WorkingDir=Workbooks(1).Path
MyFile=Dir(WorkingDir & “\*.RPT”)

End Sub

Private Function ColumnLetter(ByVal vlngNum As Long) As String
If vlngNum>26 Then
   C1=0
   Do While vlngNum>26
      C1=C1+1
      vlngNum=vlngNum−26
   Loop
   Ca=Chr(64+C1)
   Cb=Chr(64+vlngNum)
Else
   Ca=vbNullString
   Cb=Chr(64+vlngNum)
End If
ColumnLetter=Ca & Cb

End Function

Finally, the JMP IN (SAS Institute) software was used to join these various data tables together using identifiers, usually the volunteer's ID number and date of blood collection. The metadata table has more than a thousand columns.

In regard to the gene-expression data, the scanned images of chips were captured and stored in Microarray Suite 5.0 (MAS 5.0) (Affymetrix) and later transported to GCOS 1.1. Signal values, which quantify the abundance of genes from intensities of probes, and detection calls, which qualify the detection of genes into present (P), marginal (M), or absent (A), were calculated in GCOS1.1 which uses the MAS5.0 algorithm. For both HG-U133A and B chips, the scaling factor and normalization value were set to 1, resulting in no scaling or normalization after generating Signal values. This allows for testing of various scaling and normalization procedures. Signals and detection calls were exported to Excel and saved as tab-delimited text files with A chips in one folder and B chips in another.

Statistical analysis. Statistical quality control and relations among metadata variables were analyzed in JMP IN and StatView (SAS). ANOVA, t-tests, and class prediction of clinical phenotypes using CBC or electropherogram data were performed in BRB-Arraytools 3.2.0 Beta (Arraytools) developed by Dr. Richard Simon and Amy Peng Lam (available through the web-site for the Biometric Research Branch, Division of Cancer Research and Diagnosis, National Cancer Institute, U.S. National Institutes of Health). Arraytools is written for analysis of gene-expression data, but here we have imported certain quantitative metadata fields, such as CBC, to be treated as ‘genes’ by Arraytools to take advantage of its class prediction algorithm.

Relations between metadata variables and gene-expression profiles were analyzed in Arraytools. To facilitate import of text files with Signals and detection calls, in-house scripts were written in R to move files of interest into a different folder and renaming and reformatting the files to be compatible with ArrayTools: (Script provided herein below)

Script for Reformatting the Files to be Compatible with ArrayTools:

```
# objects in R scaled each chip via trimmean:
# “from”: vector of DAT file names
# “sample_ID”: dataframe of renamed file names for Arraytools keyed to DAT file
names
# “t”: older, one error version of ‘sample_ID’
# “training”: Arraytools file names for the training set samples
# “rename: function to rename the DAT files in a folder to Arraytools acceptable
names
function (from,to)
  {for (i in 1:length(from))
    {file.rename (paste(from[i], “.txt”, sep = “”),paste(to[i], “.txt”, sep = “”))}
  }
#“sample_ID_only”: from “sample_ID”, but with Arraytools name column only, no
DAT files names
#“target”: set value to scale to
#“training_files”: similar to “training”, but no column name
#“to”: vector of Arraytools compatible file names, corresponding to “from” DAT
names
# rewrite: function to reformat GCOS CHP files exported to excel
# saved as tab delimited file text file to be compatible with Arraytools
function(to)
  {for (i in 1:length(to))
    {tempfile <- read.table(paste(to[i], “.txt”, sep = “”), sep = “\t”, header = TRUE);
    names(tempfile) <- c(“Probe Set Name”, “Signal”, “Detection”);
    write.table (tempfile, file = paste(to[i], “.txt”, sep = “”), sep = “\t”, quote = FALSE,
row.names = FALSE);
    }
  }
#select_training_set: given a list of training set file names
#move these files in the original to folder to a separate folder for Arraytools
function (training_files)
{ for (i in 1:length(training_files))
  { #file.create(paste(“C:\\Dzung on Affy3\\files for R conversion\\test training set\\”,
training_files[i],“.txt”, sep = “”));
    file.copy (paste(“C:\\Dzung on Affy3\\files for R conversion\\reformated B chips
text files no scaling or normalization\\”, training_files[i], “_B.txt”, sep = “”),
        paste(“C:\\Dzung on Affy3\\files for R conversion\\test training set\\”,
training_files[i],“_B.txt”, sep = “”));
  }
}
```

Selected metadata fields were imported into the Experiment descriptors worksheet of Arraytools. After data import, Arraytools were used to determine differential gene expression and ontology, class prediction, and quantitative trait correlations, with, between, and/or among clinical phenotypes.

CBC data were obtained from two machines. The first partitioned the white blood cells (WBC) into only three groups: lymphocytes, monocytes, and granulocytes, while the second partitioned the WBC into five groups: lymphocytes, monocytes, neutrophils, eosinophils, and basophils. Therefore, to make CBC comparable between the two machines, the following in-silico transformations were performed. Since granulocytes consist of neutrophils, eosinophils, and basophils, samples with five groups were converted to three by summing up the neutrophils, eosinophils, and basophils counts. Also, blood samples from 25 volunteers not in this study were run on both machines. Their CBC showed linear correlations between the two machines (data not shown). Therefore, linear regression equations were calculated for CBC variables between the two machines. These equations were used to normalize the CBC of the current BMT cohort.

The Degradometer 1.1 software scales the electropherograms using the spiked in marker peak (51).

Scaling was performed for gene-expression data. Since for each blood sample, the same hybridization cocktail went onto the A chip and then the B chip, concatenation of the data from the two chips together in-silico to form a virtual array would be logical and bypasses issues with analyzing the two chip types separately; also, the 100 control probe sets common between the A and B chips should detect genes to result in similar Signal distributions. Several methods were considered to concatenate the A and B chips profiles.

First, if each A and B chips were separately globally scaled to a target value of 500, then the resulting Scale Factors (SF) was significantly higher for the B chips than for A (data not shown) (t-test, p<0.0001), suggesting that generally, Signals from B chips were actually lower than from A. Confirmatory of this bias was that Signals of the 100 control genes were higher in B chips than in A after globally scaling each chip. The lower overall Signals in B are probably due to the B chip containing probesets that detect mostly low expressing genes (69). These observations suggested that the above step of globally scaling each chip was not appropriate to perform prior to concatenating data from the two array types.

Thus, another method was assessed, which was to scale all A and B chips using only the 100 control genes to a target value of 500. This resulted in stable SF over time (data not shown) and that there was no significant differences in SF among the four phenotypes of healthy, sick with adenovirus infection and convalescents, and sick without adenovirus infection (data not shown) (ANOVA, p=0.1047 A chips, p=0.1782 B chips). The 100 control genes were selected based on stability in expression from a large study of various tissue types (69); therefore, this scaling method would allow for the concatenation of corresponding A and B chips and also should remove assay variations independent of gene concentration. This scaling procedure was carried out using an in-house R script (Script provided herein below):

Script for Scaling:

```
function scaled (sample_ID_only)
  {for (i in 1:length(sample_ID_only))
    {tempfileA <- read.table(paste(“C:\\Dzung on Affy3\\hk then global
scaling\\reformated A chips text files no scaling or normalization\\”,
          sample_ID_only[i], “.txt”, sep = “”), sep = “\t”, header =
TRUE, check.names = FALSE);
    tempfileB <- read.table(paste(“C:\\Dzung on Affy3\\hk then global
scaling\\reformated B chips text files no scaling or normalization\\”,
          sample_ID_only[i], “_B.txt”, sep = “”), sep = “\t”, header =
TRUE, check.names = FALSE);
    target <- 500;
    hk_scale_factorA <- target / mean(tempfileA$Signal[69:168], trim = 0.02);
    tempfileA$Signal <- (tempfileA$Signal) * hk_scale_factorA;
    hk_scale_factorB <- target / mean(tempfileB$Signal[69:168], trim = 0.02);
    tempfileB$Signal <- (tempfileB$Signal) * hk_scale_factorB;
    #hk_scale_factors <- paste (sample_ID_only[i],“\t”, hk_scale_factorA,“\t”,
hk_scale_factorB);
    #write.table (hk_scale_factors, file = “C:\\Dzung on Affy3\\hk then global
scaling\\hk_scale_factors.txt”, append = TRUE, quote = FALSE, row.names =
FALSE);
    #virtual_chip_signals <- c(tempfileA$Signal, tempfileB$signal);
    #global_scale_factor <- target / mean(virtual_chip_signals, trim = 0.02);
    #tempfileA$Signal <- (tempfileA$Signal) * global_scale_factor;
    #tempfileB$Signal <- (tempfileB$Signal) * global_scale_factor;
    #global_scale_factor_list <- c(global_scale_factor_list, global_scale_factor);
    write.table (tempfileA, file = paste(“C:\\Dzung on Affy3\\hk then global
scaling\\HKscaled A chips\\”, sample_ID_only[i], “.txt”, sep = “”), quote = FALSE,
row.names = FALSE, sep = “\t”);
    write.table (tempfileB, file = paste(“C:\\Dzung on Affy3\\hk then global
scaling\\HKscaled B chips\\”, sample_ID_only[i], “_B.txt”, sep = “”), quote = FALSE,
row.names = FALSE, sep = “\t”);
    }
  }
#above is for generating scale factors for A and B chips if only the 100 house
keepking genes were used to scaled
```

After scaling using the 100 control genes, the expression profiles from corresponding A and B chips were concatenated to form virtual arrays. Furthermore, the present inventors considered globally scaling these virtual arrays to further remove assay variations. However, the SF from this procedure showed differences among the four phenotypes: highest SF in the healthy group, then convalescents, followed by the febrile group (data not shown) (ANOVA, p<0.0001). Therefore, this step was not used for the whole data set, although it might still be useful in increasing the sensitivity of detection of genes with differential expression between groups with equivalent SF, such as between sick with- versus without-adenovirus infection. These results also suggested that relatively large subsets of transcripts differ among healthy, convalescents, and febrile, while relatively small subsets of transcripts differ between sick with- and without-adenovirus. These analysis steps were also carried out using an in-house R script (Script provided herein below):

Script to Scale ‘Virtual’ Chips:

```
# to normalize A and B chips via trimmean of 100 house keeping genes, then scale
concatenated A and B chips
# (virtual chip) to ‘target’ value using the trimmean of the virtual chip signals
# input an object containing names of files for A and B chips (sample_ID_only)
function(to)
  {for (i in 1:length(sample_ID_only))
    {# read in files
    tempfileA <- read.table(paste(“C:\\Dzung on Affy3\\files for R conversion\\hk
then global scaling\\reformated A chips text files no scaling or normalization\\”,
          sample_ID_only[i], “.txt”, sep = “”), sep = “\t”, header =
TRUE, check.names = FALSE);
    tempfileB <- read.table(paste(“C:\\Dzung on Affy3\\files for R conversion\\hk
then global scaling\\reformated B chips text files no scaling or normalization\\”,
          sample_ID_only[i], “_B.txt”, sep = “”), sep = “\t”, header =
TRUE, check.names = FALSE);
    target <- 500; #set target values
    #scale chip A and B signal via trimmean of 100 house keeping genes
    hk_scale_factorA <- target / mean(tempfileA$Signal[69:168], trim = 0.02);
    tempfileA$Signal <- (tempfileA$Signal) * hk_scale_factorA;
    hk_scale_factorB <- target / mean(tempfileB$Signal[69:168], trim = 0.02);
    tempfileB$Signal <- (tempfileB$Signal) * hk_scale_factorB;
    #scale virtual chip signals
    virtual_chip_signals <- c(tempfileA$Signal, tempfileB$signal);
    global_scale_factor <- target / mean(virtual_chip_signals, trim = 0.02);
    tempfileA$Signal <- (tempfileA$Signal) * global_scale_factor;
    tempfileB$Signal <- (tempfileB$Signal) * global_scale_factor;
    #output scaled files to different folder
    write.table (tempfileA, file = paste(“C:\\Dzung on Affy3\\files for R
conversion\\hk then global scaling\\scaled A chips\\”, sample_ID_only[i], “.txt”, sep =
“”), quote = FALSE, row.names = FALSE, sep = “\t”);
    write.table (tempfileB, file = paste(“C:\\Dzung on Affy3\\files for R
conversion\\hk then global scaling\\scaled B chips\\”, sample_ID_only[i], “_B.txt”,
sep = “”), quote = FALSE, row.names = FALSE, sep = “\t”);
    }
  }
```

Results
Quality and variations of RNA derived from PAX system from the BMTs population. Many factors contribute to the variability of target detection, with the quality of RNA being one of the most important. The quality of RNA from PAX tubes collected blood could be influenced by the disease status of the donors, sample handling, and other downstream processes. Previously, we showed that under two conditions representative of practical sample handling, the PAX system was capable of preserving blood RNA to produce good quality metrics and relatively stable transcriptome measurements (50). Recently, new RNA quality metrics have been proposed based on associations between experimental treatment of cells or purified RNA to induce RNA degradation and metrics derived from electropherograms of the RNA on the bioanalyzer (51). One new metric is the degradation factor (% Dgr/18S), which is the ratio of the average intensity of bands from degraded RNA, that is peaks of lesser molecular weight than the 18S ribosomal peak, to the 18S band intensity multiplied by 100. It is a continuous variable that is used to derive a categorical variable named ‘Alert’. Alert has five values:

BLACK—indicating that the ribosomal peaks were not detected;

NULL—no RNA degradation and corresponds to degradation factor values □8;

YELLOW—for RNA degradation can be detected and values from >8 to 16;

ORANGE—for severe degradation and values from >16 to 24;

RED—for highest alert, strong degradation, for values from >24.

The degradation factor is a more sensitive indicator of RNA degradation than the traditional 28S to 18S band intensities ratio. Another new metric is the apoptosis factor (28S/18S), which is the ratio of the height of the 28S to 18S peak and is indicative of the percentage of cells undergoing apoptosis (51). Apoptosis factors from 1 to 3 inversely correlate with 80% to 0% of cultured cells positive for annexin V. Thus, for PAX system isolated RNA from our previous study (50) and current BMTs cohort, we report the distributions of RNA quality metrics, which would be useful for comparisons and planning of protocols by other labs; determined the up-stream quality metrics that are most indicative of the quality of microarray target detection outcomes; and determined the effects of inter-individual hemoglobin variability on the sensitivity of target detection.

Electropherograms from Thach et al (50) were reanalyzed for the two PAX tube handling conditions, wherein condition E as in fresh, the RNA was extracted after the minimum incubation time of 2 hours from phlebotomy, and condition O as in frozen, the blood sat for 9 hours at room temperature followed by storage at −20° C. for 6 days, followed by RNA extraction. The degradation factor was 5.34±0.53 (mean±SE, n=6) for E and 6.53±0.40 for O with no difference between the two handling methods (Wilcoxon, p=0.13); the magnitude indicated that no degradation was detected (data not shown). Linear correlation between the degradation factor and gapdh and actin 3′/5′ is tissue dependent (51), and was not detected here (data not shown). The apoptosis factor was 1.39±0.06 for E and 1.29±0.09 for O, also with no differences between conditions (Wilcoxon, p=0.38) (data not shown). These results confirmed the lack of major differences between the handling conditions.

The reanalysis above were from samples that only have technical variation, whereas the current BMTs cohort captures inter-individual and disease states variations and has more samples; therefore, electropherograms from the BMTs were assessed. The degradation factor for the BMTs cohort was 8.47±0.47 (mean±SE, n=120) and the apoptosis factor was 1.17±0.02. The distribution of the Alerts was: 77 NULL, 36 YELLOW, 3 ORANGE, and 4 RED.

A closer look at the electropherograms of ORANGE and RED samples suggested that these samples, mostly from the same run, had high degradation factors due to increased noise in the bioanalyzer rather than true RNA degradation. In contrast to the reanalysis of Condition E and O samples above, linear correlations were detected between the degradation factor and gapdh and actin 3′/5′, probably because of greater variation and larger number of samples. However, the magnitudes of the correlations were modest (A chips gapdh r=0.526, actin r=0.303; B chips gapdh r=0.325, actin r=0.284). There was no significant correlation between 28S to 18S band intensity ratio versus degradation factor, gapdh 3′/5′, or actin 3′/5′. Also, only about 50% of the 28S to 18S band intensity ratio values derived from the bioanalyzer software fell between the 1.8 and 2.1 range, while the rest fell outside of this standard range.

Finally, the distribution of yields of total RNA as determined by the bioanalyzer ranges from 1 to 15 μg per PAX tube. These results suggest that of the metrics relating to RNA quality obtained at the bioanalyzer step: RNA yield, 28S to 18S band intensity ratio, degradation factor, and Alert, the variable Alert would be most useful in assessment of individual RNA samples for continuation of processing, as the other metrics had large variation outside of the traditional range, although microarrays with acceptable quality metrics were still obtained from those RNA samples.

In condition O, the frozen time was 6 day; whereas in the current BMT study, samples were frozen at −20° C. for up to 20 days, and a few samples had been frozen and thawed a couple of times. Therefore, to determine if frozen time and freeze-thaw affected RNA quality derived from PAX system, linear correlations were performed between the time the samples were frozen before RNA extraction and RNA quality metrics. There was no significant correlation detected between frozen time versus degradation factor, apoptosis factor, total RNA yield per PAX tube, 28S to 18S band intensity ratio, gapdh and actin 3′/5′. These results suggest that RNA derived from PAX system is stable over these conditions.

Many factors affect number of present calls, an indication of sensitivity of detection of targets. One obvious factor is average background. As average background increases, then number of present calls decrease. This was observed in the current data set, but the effect was minor (A chips, r=−0.397, p=0.00003; B chips, r=−0.211, p=0.032). A less obvious factor affecting sensitivity is the percent of globin transcripts of the mRNA population. When increasing amounts of globin mRNA transcripts were spiked into total RNA from cell line, the percent present calls decreases linearly (20). To determine if this effect is present and to quantitate its magnitude in the current data set, linear correlation was performed between Number Present and Mean Cell Hemoglobin (MCH), a measurement of picograms of hemoglobin per red blood cell that is likely to be directly related to globin mRNA amounts. A significant although minor effect was detected (r=0.229, p=0.020), but only for the B chips only. The equation of the regression line suggested that for every picogram increase in hemoglobin, there is a loss in present detection calls of 100 genes, or about 2% of the average number of present call genes detected on the B chips.

These results suggested that the quality of RNA from PAX tubes collected blood of the BMT population with various disease phenotypes and handling conditions are of good and reproducible quality for gene-expression analysis, although variation in hemoglobin amounts contributed a minor effect to the sensitivity of detection of target by the Genechip microarray. The Alert metric seemed to be a robust indicator for continuation to the target preparation steps, with values of NULL and YELLOW indicating acceptable microarray results.

Quality of microarray measurements of PAX system derived RNA from the BMTs population. The numbers of arrays processed and their allocations were determined. A total of 145 A and B chip sets were processed from hybridization cocktail samples from PAX system derived RNA. Of these, 128 were from the BMTs, and the remaining 17 were from civilians.

Of the 17, 6 were from the same donor and were samples used in the condition O versus E study (50); 6 were from another donor to compare using total versus poly A RNA; 2 were technical replicates from a third donor; and 3 were technical replicates from a female donor.

The 128 chips sets from the BMTs were run in 10 batches (variable name ‘RNA to hyb cocktail Batch #’). Batch 1 had 8 blood samples and polyA RNA was used as in Thach et al. (50). Batch 2 had 12 chip sets with 8 blood samples that were processed as in Batch 1, but the RNA was over fragmented; four of these samples had more than 5 μg of cRNA left over, so these were hybridized to the arrays resulting in the 12 chip sets for Batch 2. Batch 3 also had 12 chip sets with 8 blood samples that were processed using total RNA; 4 of the eight blood samples yielded enough total RNA to have duplicates using polyA RNA instead. The remaining batches totaling 96 chip sets were processed as the 8 total RNA blood samples from Batch 3. One of the 96 chip sets was from a convalescent BMT whose nasal wash still had positive adenoviral culture; therefore, this singular case was excluded from most analysis. The resulting 95 chip sets were used as the training set in class prediction analysis. The other 50 chip sets, regardless of processing differences were placed into the test set. The 95 chips sets and the 8 from Batch 3 summed to 103 chip sets that were processed similarly, and these 103 chip sets were used for most other analysis such as class comparisons. Each batch had about equal representation of the four phenotypes: healthy, febrile with adenovirus and convalescents, and febrile without adenovirus. Therefore, comparisons among these four groups should detect biological differences as these four groups have similar variations due to processing. These results above are summarized in Table 5 below:

TABLE 5

| batch | | | febrile w/ | febrile w/o | |
|---|---|---|---|---|---|
| number | Convalescents | healthy | adenvirus | adenovirus | total |
| 10 | 3 | 3 | 3 | 1 | 10 |
| 3 | 2 | 2 | 2 | 2 | 8 |
| 4 | 2 | 2 | 2 | 2 | 8 |
| 5 | 2 | 2 | 2 | 2 | 8 |
| 6 | 7 | 4 | 7 | 2 | 20 |
| 7 | 5 | 8 | 4 | 1 | 18 |
| 8 | 3 | 4 | 4 | 4 | 15 |
| 9 | 3 | 4 | 4 | 5 | 16 |
| total | 27 | 29 | 28 | 19 | 103 |

The correlation of signals and concentrations and the sensitivity of the bioB, bioC, bioD, and cre cRNA spike-ins were evaluated. The spike-ins showed strong linear relationship with known concentration across all chips (data not shown) and that the percent present calls of bioB, whose concentration is at the level of assay sensitivity, was 100% of the time suggesting good sensitivity for all the chips. After scaling via 100 control genes, the spike-ins still showed strong linear relationship with known concentration, suggesting that the scaling procedure did not introduce significant artifacts (data not shown).

Individual control charts versus the date the microarray was scanned were plotted to look for stability of quality metrics, to determine outliers and excluded arrays when error in processing was known, and to compare our results with values from other labs and values proposed by Affymetrix. The in silico parameter settings were uniform throughout as expected. For the A chips, there was an upward drift in background and noise due to drifting in the scanner as these metrics returned to normal after recalibration of the scanner. Most of the B chips were processed before drifting and after recalibration so this factor did not affect them. The percent present was 32±10 (average ±3SD) for A chips and 21±6 for B chips. Batch 2 had been over fragmented resulting in high gapdh and actin 3′/5′ and was excluded from analysis where appropriate. All other chips showed gapdh and actin 3′/5′ values well less than three, the limit proposed by Affymetrix (68). All quality metrics, including background and noise were stable for the 103 chip sets from identical protocol.

These QC results suggested the reliability of our process and facilitated the inclusion and exclusion of microarrays to form subsets suitable for a particular statistical analysis to answer certain questions.

Class prediction of infection status. To determine if sets of genes could classify the four phenotypes, healthy, febrile with adenovirus and convalescents, and febrile without adenovirus, class prediction on the training set was performed. For supervised class prediction, the class labels were results from the gold standard assay of culture for adenovirus from samples of the febrile and convalescent groups. Unsupervised clustering of samples suggested that the predominant variation among gene expression profiles were febrile versus non-febrile patients (not shown).

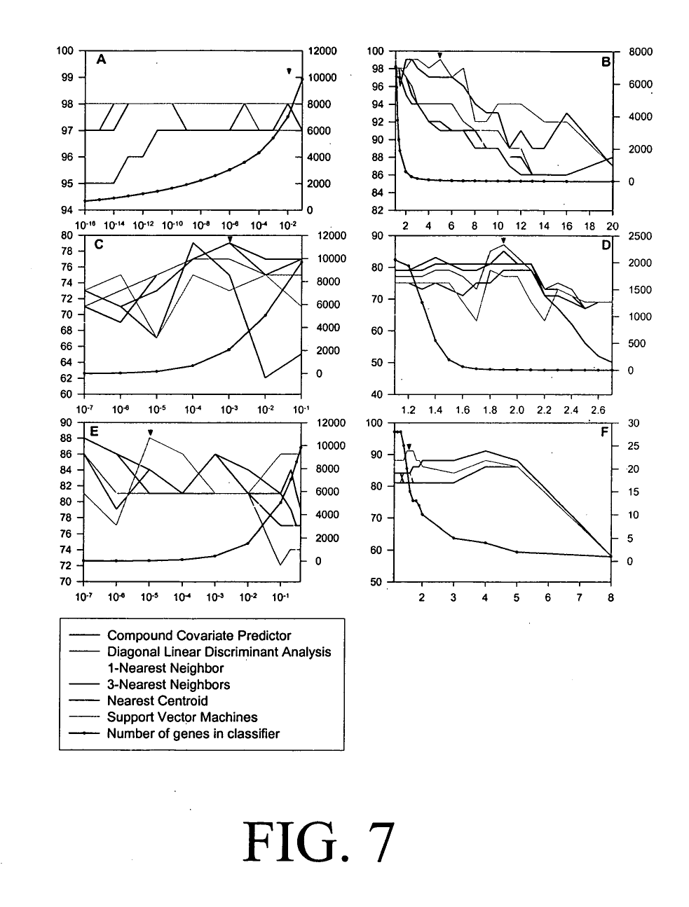

Therefore, to determine sets of genes that could best classify febrile versus non-febrile patients, febrile with adenovirus versus without, and healthy versus convalescents, class prediction was performed and optimized for these three comparisons (FIG. 7). Four parameters were varied to obtain optimal percent correct classification. One is the algorithm for classification, which consisted of six methods tested: compound covariate predictor, diagonal linear discriminant analysis, 1—nearest neighbor, 3—nearest neighbors, nearest centroid, and support vector machines. For all these six methods, the ‘univariate significant p-value cut off’ or the ‘univariate misclassification rate’ was varied. Also the effect of using the randomized variance model for univariate tests was assessed. Finally, in combination with the optimal univariate p-value or classification rate and present or absent of randomized variance model, the fold ratio of geometric means between two classes was optimized.

The optimized percent correctly classified and the optimal conditions for the three comparisons results are shown in Table 6 below:

TABLE 6

| | Classes to predict | | | Optimal parameters values | | | |
|---|---|---|---|---|---|---|---|
| | | | optimum | | univariate | | |
| | | | percent | | misclass | fold | |
| Data used | Group 1 | Group 2 | correct | algorithm | rate | change | alpha |
| gene-expression | non-febriles | febriles | 99 | SVM, NN, or | 0.05, 0.4, | 1.2, 2-3 | 0.01 |
| | | | | 3NN | 0.5 | | |
| | convalescents | healthy | 87 | DLDA | | 1.9 | 0.001 |
| | febrile w/ | febrile w/o | 91 | SVM | | 1.5-1.7 | 0.00001 |
| | adenovirus | adenovirus | | | | | |
| CBC | non-febriles | febriles | 91 | SVM | 0.2 | 1.1-1.2 | |
| | convalescents | healthy | 77 | DLDA | 0.3 | none | |
| | febrile w/ | febrile w/o | 77 | 3NN | | 1.1 | 0.1 |
| | adenovirus | adenovirus | | | | | |
| Electropherogram | non-febriles | febriles | 81 | SVM | 0.4 | 1.02 | |
| | convalescents | healthy | 67 | SVM | | 1.02 | 0.3 |
| | febrile w/ | febrile w/o | | | | | |
| | adenovirus | adenovirus | 81 | SVM | | 1.02 | 0.4 |

Also shown in the table are optimized percent correct and conditions when using CBC or electropherograms data. The results showed that under optimal conditions for each data types, gene-expression data provided information that best classified the four groups, with 99% correct between febrile versus non-febrile, 87% between healthy and convalescents, and 91% between sick with adenovirus versus without. The optimal number of genes for equal optimal classifications among the four groups tended to be nested sets, with the smallest set that gave the same optimal class prediction accuracy containing genes with the most differential expression. This was likely so because some genes are correlated with each other and thus provided equivalent amounts of information for classification. Tables 7, 10, and 11 provide the p-values as a measure of reliability of prediction and lists the minimal set of genes used to classify the following classes: febrile versus non-febrile patients—99% Feverstatus, p<5E-4, number of genes in classifier=47 (Table 7); healthy versus convalescents—87% accurate between healthy and convalescents, p=0.001, number of genes in classifier=8 (Table 10); and febrile with adenovirus versus without—91% Febriles with vs. without adenovirus infection, p <5E-4, number of genes in classifier=11 (Table 11).

TABLE 7
Minimal Set Of Genes Used To Classify Febrile Versus Non-Febrile Patients (Sorted by T-value)

| | | | | Geom | Geom | | | | |
|---|---|---|---|---|---|---|---|---|---|
| | | | | mean | mean | | | | Gene |
| | t-value | Parametric p-value | % CV support | of intensities in class 1: H | of intensities in class 2: S | Probe set | Chip # | Description | symbol |
| 1 | −22.56 | p < 0.000001 | 100 | 64 | 495.9 | 227458_at | Chip ‘B’ | programmed cell death 1 ligand 1 | PDCD1LG1 |
| 2 | −22.03 | p < 0.000001 | 100 | 220.4 | 1320.5 | 202446_s_at | Chip ‘A’ | phospholipid scramblase 1 | PLSCR1 |
| 3 | −21.68 | p < 0.000001 | 100 | 93.1 | 611.4 | 216950_s_at | Chip ‘A’ | Fc fragment of IgG, high affinity Ia, receptor for | FCGR1A |
| | | | | | | | | (CD64) /// Fc fragment of IgG, high affinity Ia, | |
| | | | | | | | | receptor for (CD64) | |
| 4 | −20.81 | p < 0.000001 | 100 | 96.1 | 490.5 | 202430_s_at | Chip ‘A’ | phospholipid scramblase 1 | PLSCR1 |
| 5 | −20.73 | p < 0.000001 | 100 | 117.3 | 779 | 214511_x_at | Chip ‘A’ | Fc fragment of IgG, high affinity Ia, receptor for | FCGR1A |
| | | | | | | | | (CD64) | |
| 6 | −18.07 | p < 0.000001 | 100 | 73.3 | 389.5 | 209498_at | Chip ‘A’ | carcinoembryonic antigen-related cell adhesion | CEACAM1 |
| | | | | | | | | molecule 1 (biliary glycoprotein) | |
| 7 | −16.48 | p < 0.000001 | 100 | 56.3 | 557.4 | 200986_at | Chip ‘A’ | serine (or cysteine) proteinase inhibitor, clade G | SERPING1 |
| | | | | | | | | (C1 inhibitor), member 1, (angioedema, | |
| | | | | | | | | hereditary) /// serine (or cysteine) proteinase | |
| | | | | | | | | inhibitor, clade G (C1 inhibitor), member 1, | |
| | | | | | | | | (angiodema, hereditary) | |
| 8 | −15.62 | p < 0.000001 | 100 | 69.2 | 374.3 | 206025_s_at | Chip ‘A’ | tumor necrosis factor, alpha-induced protein 6 | TNFAIP6 |
| 9 | −15.52 | p < 0.000001 | 100 | 28 | 199 | 238439_at | Chip ‘B’ | ankyrin repeat domain 22 | ANKRD22 |
| 10 | −15.4 | p < 0.000001 | 100 | 148.1 | 947.6 | 227609_at | Chip ‘B’ | epithelial stromal interaction 1 (breast) | EPSTI1 |
| 11 | −15.3 | p < 0.000001 | 91 | 81.8 | 413.6 | 230036_at | Chip ‘B’ | hypothetical protein FLJ39885 | FLJ39885 |
| 12 | −15.01 | p < 0.000001 | 100 | 34.1 | 235 | 222154_s_at | Chip ‘A’ | DNA polymerase-transactivated protein 6 | DNAPTP6 |
| 13 | −14.58 | p < 0.000001 | 100 | 86 | 459.5 | 209417_s_at | Chip ‘A’ | interferon-induced protein 35 | IFI35 |
| 14 | −14.46 | p < 0.000001 | 100 | 60.8 | 368.9 | 205552_s_at | Chip ‘A’ | 2′,5′-oligoadenylate synthetase 1, 40/46 kDa /// | OAS1 |
| | | | | | | | | 2′,5′-oligoadenylate synthetase 1, 40/46 kDa | |
| 15 | −14.21 | p < 0.000001 | 100 | 66.1 | 484.4 | 219669_at | Chip ‘A’ | polycythemia rubra vera 1 /// polycythemia rubra | PRV1 |
| | | | | | | | | vera 1 | |
| 16 | −14.15 | p < 0.000001 | 100 | 3.8 | 32.8 | 204068_at | Chip ‘A’ | serine/threonine kinase 3 (STE20 homolog, yeast) | STK3 |
| | | | | | | | | /// serine/threonine kinase 3 (STE20 homolog, | |
| | | | | | | | | yeast) | |
| 17 | −14.02 | p < 0.000001 | 100 | 190.1 | 974.4 | 202269_x_at | Chip ‘A’ | guanylate binding protein 1, interferon-inducible, | GBP1 |
| | | | | | | | | 67 kDa | |
| 18 | −13.65 | p < 0.000001 | 100 | 86.9 | 527.7 | 202270_at | Chip ‘A’ | guanylate binding protein 1, interferon-inducible, | GBP1 |
| | | | | | | | | 67 kDa /// guanylate binding protein 1, interferon- | |
| | | | | | | | | inducible, 67 kDa | |
| 19 | −13.58 | p < 0.000001 | 100 | 143.7 | 996.8 | 231577_s_at | Chip ‘B’ | guanylate binding protein 1, interferon-inducible, | GBP1 |
| | | | | | | | | 67 kDa | |
| 20 | −13.41 | p < 0.000001 | 100 | 13.8 | 90.1 | 207500_at | Chip ‘A’ | caspase 5, apoptosis-related cysteine protease /// | CASP5 |
| | | | | | | | | caspase 5, apoptosis-related cysteine protease | |
| 21 | −13.23 | p < 0.000001 | 100 | 353.8 | 1987.5 | 229450_at | Chip ‘B’ | interferon-induced protein with tetratricopeptide | IFIT4 |
| | | | | | | | | repeats 4 | |
| 22 | −13.18 | p < 0.000001 | 100 | 45.5 | 260.3 | 206637_at | Chip ‘A’ | G protein-coupled receptor 105 | GPR105 |
| 23 | −13.14 | p < 0.000001 | 100 | 11.4 | 144.1 | 228439_at | Chip ‘B’ | hypothetical protein BC012330 | MGC20410 |
| 24 | −13.09 | p < 0.000001 | 100 | 59.8 | 1137.5 | 242625_at | Chip ‘B’ | viperin | cig5 |
| 25 | −12.38 | p < 0.000001 | 100 | 74.8 | 783.5 | 226702_at | Chip ‘B’ | hypothetical protein LOC129607 | LOC129607 |
| 26 | −12.34 | p < 0.000001 | 100 | 43.8 | 239.1 | 214453_s_at | Chip ‘A’ | interferon-induced protein 44 /// interferon- | IFI44 |
| | | | | | | | | induced protein 44 | |
| 27 | −12.32 | p < 0.000001 | 100 | 72.8 | 435.1 | 238581_at | Chip ‘B’ | guanylate binding protein 5 | GBP5 |
| 28 | −12.2 | p < 0.000001 | 100 | 14.9 | 82.8 | 225353_s_at | Chip ‘B’ | complement component 1, q subcomponent, | C1QG |
| | | | | | | | | gamma polypeptide | |
| 29 | −12.07 | p < 0.000001 | 100 | 82.9 | 445.5 | 228617_at | Chip ‘B’ | XIAP associated factor-1 | HSXIAPAF1 |
| 30 | −11.93 | p < 0.000001 | 100 | 33.7 | 703.8 | 213797_at | Chip ‘A’ | viperin | cig5 |
| 31 | −11.86 | p < 0.000001 | 100 | 37.6 | 207.3 | 203234_at | Chip ‘A’ | uridine phosphorylase 1 /// uridine phosphorylase 1 | UPP1 |
| 32 | −11.68 | p < 0.000001 | 100 | 16.2 | 178 | 211012_s_at | Chip ‘A’ | promyelocytic leukemia | PML |
| 33 | −11.67 | p < 0.000001 | 100 | 18.9 | 113 | 205569_at | Chip ‘A’ | lysosomal-associated membrane protein 3 /// | LAMP3 |
| | | | | | | | | lysosomal-associated membrane protein 3 | |
| 34 | −11.67 | p < 0.000001 | 100 | 24.9 | 206.2 | 219684_at | Chip ‘A’ | 28 kD interferon responsive protein /// 28 kD | IFRG28 |
| | | | | | | | | interferon responsive protein | |
| 35 | −11.27 | p < 0.000001 | 100 | 177.7 | 1219.3 | 205483_s_at | Chip ‘A’ | interferon, alpha-inducible protein (clone IFI- | G1P2 |
| | | | | | | | | 15K) /// interferon, alpha-inducible protein (clone | |
| | | | | | | | | IFI-15K) | |
| 36 | −10.96 | p < 0.000001 | 100 | 27.6 | 408.8 | 204439_at | Chip ‘A’ | chromosome 1 open reading frame 29 /// | C1orf29 |
| | | | | | | | | chromosome 1 open reading frame 29 | |
| 37 | −10.76 | p < 0.000001 | 98 | 25.4 | 129.9 | 214059_at | Chip ‘A’ | interferon-induced protein 44 | IFI44 |
| 38 | −10.69 | p < 0.000001 | 100 | 59.2 | 391 | 229390_at | Chip ‘B’ | Full length insert cDNA clone ZA84A12 | |
| 39 | −10.61 | p < 0.000001 | 100 | 10.2 | 106.9 | 236156_at | Chip ‘B’ | lipase A, lysosomal acid, cholesterol esterase | LIPA |
| | | | | | | | | (Wolman disease) | |
| 40 | −10.56 | p < 0.000001 | 100 | 90.6 | 617 | 202869_at | Chip ‘A’ | 2′,5′-oligoadenylate synthetase 1, 40/46 kDa | OAS1 |
| 41 | −9.98 | p < 0.000001 | 100 | 241.4 | 1315 | 202086_at | Chip ‘A’ | myxovirus (influenza virus) resistance 1, | MX1 |
| | | | | | | | | interferon-inducible protein p78 (mouse) /// | |
| | | | | | | | | myxovirus (influenza virus) resistance 1, | |
| | | | | | | | | interferon-inducible protein p78 (mouse) | |
| 42 | −9.96 | p < 0.000001 | 100 | 33.3 | 178.7 | 229391_s_at | Chip ‘B’ | Full length insert cDNA clone ZA84A12 | |
| 43 | −9.91 | p < 0.000001 | 100 | 6.5 | 54.4 | 219519_s_at | Chip ‘A’ | sialoadhesin | SN |
| 44 | −9.77 | p < 0.000001 | 100 | 18 | 105.6 | 206133_at | Chip ‘A’ | XIAP associated factor-1 /// XIAP associated | HSXIAPAF1 |
| | | | | | | | | factor-1 | |
| 45 | −9.33 | p < 0.000001 | 100 | 22.3 | 346.4 | 203153_at | Chip ‘A’ | interferon-induced protein with tetratricopeptide | IFIT1 |
| | | | | | | | | repeats 1 /// interferon-induced protein with | |
| | | | | | | | | tetratricopeptide repeats 1 | |
| 46 | −8.61 | p < 0.000001 | 100 | 20.3 | 109.8 | 206553_at | Chip ‘A’ | 2′-5′-oligoadenylate synthetase 2, 69/71 kDa | OAS2 |
| 47 | −8.48 | p < 0.000001 | 100 | 14.9 | 123 | 202411_at | Chip ‘A’ | interferon, alpha-inducible protein 27 /// | IFI27 |
| | | | | | | | | interferon, alpha-inducible protein 27 | |

From the genes listed above, a table of ‘Observed v. Expected’ table of GO classes and parent classes, in list of 47 genes shown above can be prepared to help elucidate the molecular function (Table 8) and/or biological processes (Table 9) in which the identified genes take part. Only GO classes and parent classes with at least 5 observations in the selected subset and with an ‘Observed vs. Expected’ ratio of at least 2 are shown.

TABLE 8
Molecular Function

| | | Observed in | Expected in | |
|---|---|---|---|---|
| | | selected | selected | Observed/ |
| GO id | GO classification | subset | subset | Expected |
| 0005525 | GTP binding | 5 | 0.83 | 5.99 |
| 0019001 | guanyl nucleotide | 5 | 0.84 | 5.92 |
| | binding | | | |
| 0017076 | purine nucleotide | 8 | 3.59 | 2.23 |
| | binding | | | |
| 0000166 | nucleotide binding | 8 | 3.62 | 2.21 |

TABLE 9
Biological Process

| | | Observed in | Expected in | |
|---|---|---|---|---|
| | | selected | selected | Observed/ |
| GO id | GO classification | subset | subset | Expected |
| 0009615 | response to virus | 5 | 0.15 | 32.44 |
| 0006955 | immune response | 20 | 2.6 | 7.69 |
| 0009607 | response to biotic | 22 | 3.08 | 7.14 |
| | stimulus | | | |
| 0006952 | defense response | 20 | 2.81 | 7.12 |
| 0009613 | response to pest\, | 9 | 1.45 | 6.21 |
| | pathogen or parasite | | | |
| 0043207 | response to external | 9 | 1.47 | 6.13 |
| | biotic stimulus | | | |
| 0050874 | organismal | 22 | 3.88 | 5.67 |
| | physiological | | | |
| process | | | | |
| 0050896 | response to stimulus | 22 | 4.89 | 4.5 |
| 0009605 | response to external | 9 | 2.49 | 3.61 |
| | stimulus | | | |
| 0006950 | response to stress | 9 | 2.58 | 3.49 |

TABLE 10
Minimal Set Of Genes Used To Classify Healthy Versus Convalescent Patients (Sorted by T-value)

| | | | | Geom mean | Geom mean | | | | |
|---|---|---|---|---|---|---|---|---|---|
| | | Parametric | % CV | of intensities in | of intensities in | | | | Gene |
| | t-value | p-value | support | class 1: F_NE | class 2: H_ND | Probe set | Chip # | Description | symbol |
| 1 | −4.61 | 2.8e−05 | 100 | 12.8 | 27.8 | 213642_at | Chip ‘A’ | ribosomal protein L27 | RPL27 |
| 2 | −4.27 | 8.8e−05 | 100 | 24.3 | 63.4 | 213941_x_at | Chip ‘A’ | ribosomal protein S7 | RPS7 |
| 3 | 4.04 | 0.000185 | 87 | 39.6 | 20.3 | 201280_s_at | Chip ‘A’ | disabled homolog 2, mitogen- | DAB2 |
| | | | | | | | | responsive phosphoprotein | |
| | | | | | | | | (Drosophila) | |
| 4 | 4.13 | 0.000139 | 100 | 19.3 | 8.9 | 205116_at | Chip ‘A’ | laminin, alpha 2 (merosin, congenital | LAMA2 |
| | | | | | | | | muscular dystrophy) /// laminin, | |
| | | | | | | | | alpha 2 (merosin, congenital | |
| | | | | | | | | muscular dystrophy) | |
| 5 | 4.13 | 0.000138 | 100 | 182 | 75.1 | 213674_x_at | Chip ‘A’ | immunoglobulin heavy constant mu | IGHM |
| 6 | 4.2 | 0.000108 | 100 | 67.4 | 22.5 | 215621_s_at | Chip ‘A’ | immunoglobulin heavy constant mu | IGHM |
| 7 | 4.57 | 3.3e−05 | 100 | 13.6 | 6.7 | 203780_at | Chip ‘A’ | epithelial V-like antigen 1 /// | EVA1 |
| | | | | | | | | epithelial V-like antigen 1 | |
| 8 | 4.71 | 2e−05 | 98 | 103.5 | 51.5 | 227250_at | Chip ‘B’ | kringle containing transmembrane | KREMEN1 |
| | | | | | | | | protein 1 | |

TABLE 11
Minimal Set Of Genes Used To Classify Febrile With Adenovirus Versus Febrile Without Adenovirus Patients (Sorted by T-value)

| | | | | Geom mean | Geom mean | | | | |
|---|---|---|---|---|---|---|---|---|---|
| | t- | Parametric p- | % CV | of intensities in | of intensities in | | | | |
| | value | value | support | class 1: S_AD | class 2: S_NE | Probe set | Chip # | Description | Gene symbol |
| 1 | −5.15 | 7e−06 | 53 | 47.5 | 118.4 | 205227_at | Chip ‘A’ | interleukin 1 receptor accessory | IL1RAP |
| | | | | | | | | protein /// interleukin 1 receptor | |
| | | | | | | | | accessory protein | |
| 2 | 5.18 | 6e−06 | 60 | 198.3 | 100 | 219062_s_at | Chip ‘A’ | zinc finger, CCHC domain | ZCCHC2 |
| | | | | | | | | containing 2 | |
| 3 | 5.39 | 3e−06 | 100 | 356.9 | 129.5 | 214453_s_at | Chip ‘A’ | interferon-induced protein 44 /// | IFI44 |
| | | | | | | | | interferon-induced protein 44 | |
| 4 | 5.39 | 3e−06 | 100 | 54.2 | 12.3 | 233425_at | Chip ‘B’ | zinc finger, CCHC domain | ZCCHC2 |
| | | | | | | | | containing 2 | |
| 5 | 5.4 | 3e−06 | 100 | 26 | 11.8 | 218548_x_at | Chip ‘A’ | putative secreted protein | ZSIG11 |
| | | | | | | | | ZSIG11 /// putative | |
| | | | | | | | | secreted protein | |
| | | | | | | | | ZSIG11 | |
| 6 | 5.43 | 3e−06 | 100 | 30.7 | 13.1 | 223096_at | Chip ‘B’ | nucleolar protein NOP5/NOP58 | NOP5/NOP58 |
| 7 | 5.73 | 1e−06 | 100 | 136.1 | 42 | 200923_at | Chip ‘A’ | lectin, galactoside-binding, | LGALS3BP |
| | | | | | | | | soluble, 3 binding protein /// | |
| | | | | | | | | lectin, galactoside-binding, | |
| | | | | | | | | soluble, 3 binding protein | |
| 8 | 5.9 | p < 0.000001 | 100 | 354.3 | 180.2 | 223343_at | Chip ‘B’ | membrane-spanning 4-domains, | MS4A7 |
| | | | | | | | | subfamily A, member 7 | |
| 9 | 6.24 | p < 0.000001 | 100 | 1128.4 | 226 | 202145_at | Chip ‘A’ | lymphocyte antigen 6 complex, | LY6E |
| | | | | | | | | locus E /// lymphocyte antigen 6 | |
| | | | | | | | | complex, locus E | |
| 10 | 6.5 | p < 0.000001 | 100 | 116.4 | 64.6 | 204821_at | Chip ‘A’ | butyrophilin, subfamily 3, | BTN3A3 |
| | | | | | | | | member A3 | |
| 11 | 6.54 | p < 0.000001 | 100 | 283.4 | 34.3 | 202411_at | Chip ‘A’ | interferon, alpha-inducible | IFI27 |
| | | | | | | | | protein 27 /// interferon, | |
| | | | | | | | | alpha-inducible | |
| | | | | | | | | protein 27 | |

Categorical and continuous metadata variables co-varying with the four phenotypes above were assessed. The only categorical variables that correlated with the four phenotypes involved the lots of the PAX system used. These covariates were unlikely to affect gene expression outcomes because the manufacturers have QC their products for consistency. ‘Perceived Stress’ showed increasing qualitative trend with sickness, but this was expected. This increase our confidence that our class prediction set of genes is due to infection health status rather than other confounding variables.

Tables 18, 22, and 26 provide a larger list of genes that still give high percent correct classification, in order of: febrile versus non-febrile patients, febrile with adenovirus versus without adenovirus patients, and healthy versus convalescent patients, respectively. In Tables 18, 22, and 26, the composition of classifiers is listed for genes significant at the 0.001 level and is sorted by t-value.

Tables 16, 20, and 24 provide a detailed summary for the performance of classifiers during cross-validation used for Tables 18, 22, and 26.

Tables 17, 21, and 25 provide further details as to the performance of classifiers during cross-validation with respect to Performance of the Compound Covariate Predictor Classifier, Performance of the 1—Nearest Neighbor Classifier, Performance of the 3—Nearest Neighbors Classifier, Performance of the Nearest Centroid Classifier, Performance of the Support Vector Machine Classifier, and Performance of the Linear Diagonal Discriminant Analysis Classifier. Specifically, Tables 17, 21, and 25 reports the parameters used for each classification method and each class.

For compilation of the data in Tables 17, 21, and 25, the following formulae were employed:

Let, for some class A,
n11=number of class A samples predicted as A
n12=number of class A samples predicted as non-A
n21=number of non-A samples predicted as A
n22=number of non-A samples predicted as non-A

Then the following parameters can characterize performance of classifiers:
Sensitivity=n11/(n11+n12)
Specificity=n22/(n21+n22)
Positive Predictive Value (PPV)=n11/(n11+n21)
Negative Predictive Value (NPV)=n22/(n12+n22)

Tables 19, 23, and 27 provides a table of ‘Observed v. Expected’ table of GO classes and parent classes, and lists the frequency of genes reported in Tables 18, 22, and 26 to help elucidate the cellular component, molecular function and/or biological processes in which the identified genes take part. Only GO classes and parent classes with at least 5 observations in the selected subset and with an ‘Observed vs. Expected’ ratio of at least 2 are shown.

Class comparisons. To determine lists of genes that are differentially expressed among the four phenotypes, class comparisons were performed. Tables 28, 30, and 32 show the list of genes found to be different between febrile versus non-febrile patients, febrile with adenovirus versus without, and healthy versus convalescents, respectively. Tables 29, 31, and 33 provide a table of ‘Observed v. Expected’ table of GO classes and parent classes, and lists the frequency of genes reported in Tables 28, 30, and 32 to help elucidate the cellular component, molecular function and/or biological processes in which the identified genes take part. Only GO classes and parent classes with at least 5 observations in the selected subset and with an ‘Observed vs. Expected’ ratio of at least 2 are shown.

For Table 28

Description of the Problem:

Number of classes: 2

Number of genes: 44928

Number of genes that passed filtering criteria: 15720

Type of univariate test used: Two-sample T-test (with random variance model)

Column of the Experiment Descriptors sheet that defines class variable: Fever status

Multivariate Permutations test was computed based on 1000 random permutations

Nominal significance level of each univariate test: 0.001

Confidence level of false discovery rate assessment: 90%

Maximum allowed number of false-positive genes: 10

Maximum allowed proportion of false-positive genes: 0.1

Summary of Results:

Number of genes significant at 0.001 level of the univariate test: 5768

Probability of getting at least 5768 genes significant by chance (at the 0.001 level) if there are no real differences between the classes: 0

Genes which Discriminate Among Classes:

Table 28—Sorted by p-value of the univariate test.

The first 5768 genes are significant at the nominal 0.001 level of the univariate test

With probability of 90% the first 5142 genes contain no more than 10 false discoveries.

With probability of 90% the first 6430 genes contain no more than 10% of false discoveries. Further extension of the list was halted because the list would contain more than 100 false discoveries

For Table 30

Description of the Problem:

Number of classes: 2

Number of genes: 44928

Number of genes that passed filtering criteria: 15720

Type of univariate test used: Two-sample T-test (with random variance model)

Column of the Experiment Descriptors sheet that defines class variable : H_ND vs. F_NE only

Multivariate Permutations test was computed based on 1000 random permutations

Nominal significance level of each univariate test: 0.001

Confidence level of false discovery rate assessment: 90%

Maximum allowed number of false-positive genes: 10

Maximum allowed proportion of false-positive genes: 0.1

Summary of Results:

Number of genes significant at 0.001 level of the univariate test: 2943

Probability of getting at least 2943 genes significant by chance (at the 0.001 level) if there are no real differences between the classes: 0

Genes Which Discriminate Among Classes:

Table 30—Sorted by p-value of the univariate test.

The first 2943 genes are significant at the nominal 0.001 level of the univariate test

With probability of 90% the first 2151 genes contain no more than 10 false discoveries.

With probability of 90% the first 4562 genes contain no more than 10% of false discoveries. Further extension of the list was halted because the list would contain more than 100 false discoveries

For Table 32

Description of the Problem:

Number of classes: 2

Number of genes: 44928

Number of genes that passed filtering criteria: 15720

Type of univariate test used: Two-sample T-test (with random variance model)

Column of the Experiment Descriptors sheet that defines class variable : S_AD vs. S_NE only

Multivariate Permutations test was computed based on 1000 random permutations

Nominal significance level of each univariate test: 0.001

Confidence level of false discovery rate assessment: 90%

Maximum allowed number of false-positive genes: 10

Maximum allowed proportion of false-positive genes: 0.1

Summary of Results:

Number of genes significant at 0.001 level of the univariate test: 44

Probability of getting at least 445 genes significant by chance (at the 0.001 level) if there are no real differences between the classes: 0.001

Genes Which Discriminate Among Classes:

Table 32—Sorted by p-value of the univariate test.

The first 445 genes are significant at the nominal 0.001 level of the univariate test

With probability of 90% the first 229 genes contain no more than 10 false discoveries.

With probability of 90% the first 758 genes contain no more than 10% of false discoveries.

However, because of differences in CBC (Table 12 below), these differences in RNA could be due to cell type heterogeneity and/or differential expression at the per cell level. Although large expression differences are likely to be due to differential expression at the per cell level because the differences in CBC variables cannot likely to account for these large differences. Statistical models would have to be developed to sort out these two effects. Serendipitously, there were no differences in CBC for comparisons between febrile with adenovirus versus without (Table 12 below).

TABLE 12

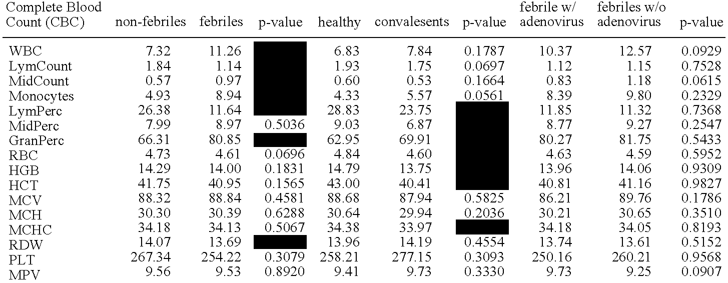

Differences in CBC between non-febriles versus febriles, healthy versus convalescents, but not between febriles with versus without adenovirus.
P-value columns are from Wilcoxon testing for differences in CBC variables between the groups. Highlights indicate significant differences.

Therefore, one could surmise that the differentially expressed genes were at the per cell level, suggesting that the biomolecular pathways involving these genes are involved in differences between adenovirus infection and non-adenovirus infection. To determine these pathways, the gene list was integrated with the KEGG pathway and the Genetic Association databases using EASE (70) to elucidate the functions of these genes in known pathways.

The results for the KEGG pathway database search are as follows:

□ hsa00071 Fatty acid metabolism

2180 ACSL1; acyl-CoA synthetase long-chain family member 1 [EC:6.2.1.3] [SP:LCF1_HUMAN]

51703 ACSL5; acyl-CoA synthetase long-chain family member 5 [EC:6.2.1.3] [SP:LCF5_HUMAN]

□ hsa00190 Oxidative phosphorylation

1355 COX15; COX15 homolog, cytochrome c oxidase assembly protein (yeast)

522 ATP5J; ATP synthase, H+transporting, mitochondrial FO complex, subunit F6 [EC:3.6.3.14] [SP:ATPR_HUMAN]

□ hsa00193 ATP synthesis

522 ATP5J; ATP synthase, H+transporting, mitochondrial FO complex, subunit F6 [EC:3.6.3.14] [SP:ATPR_HUMAN]

□ hsa00230 Purine metabolism

3614 IMPDH1; IMP (inosine monophosphate) dehydrogenase 1 [EC:1.1.1.205] [SP:IMD1_HUMAN]

6241 RRM2; ribonucleotide reductase M2 polypeptide [EC:1.17.4.1] [SP:RIR2_HUMAN]

953 ENTPD1; ectonucleoside triphosphate diphosphohydrolase 1 [EC:3.6.1.5] [SP:ENP1_HUMAN]

□ hsa00240 Pyrimidine metabolism

6241 RRM2; ribonucleotide reductase M2 polypeptide [EC:1.17.4.1] [SP:RIR2_HUMAN]

7298 TYMS; thymidylate synthetase [EC:2.1.1.45] [SP:TYSY_HUMAN]

953 ENTPD1; ectonucleoside triphosphate diphosphohydrolase 1 [EC:3.6.1.5] [SP:ENP1_HUMAN]

□ hsa00252 Alanine and aspartate metabolism

1615 DARS; aspartyl-tRNA synthetase [EC:6.1.1.12] [SP:SYD_HUMAN]

□ hsa00361 gamma-Hexachlorocyclohexane degradation

93650 ACPT; acid phosphatase, testicular [EC:3.1.3.2]

□ hsa00510 N-Glycans biosynthesis

6185 RPN2; ribophorin II [EC:2.4.1.119] [SP:RIB2_HUMAN]

□ hsa00532 Chondroitin/Heparan sulfate biosynthesis

55501 CHST12; carbohydrate (chondroitin 4) sulfotransferase 12

□ hsa00561 Glycerolipid metabolism

2710 GK; glycerol kinase [EC:2.7.1.30] [SP:GLPK_HUMAN]

□ hsa00670 One carbon pool by folate

10588 MTHFS; 5,10-methenyltetrahydrofolate synthetase (5-formyltetrahydrofolate cyclo-ligase) [EC:6.3.3.2] [SP:FTHC_HUMAN]

7298 TYMS; thymidylate synthetase [EC:2.1.1.45] [SP:TYSY_HUMAN]

□ hsa00740 Riboflavin metabolism

93650 ACPT; acid phosphatase, testicular [EC:3.1.3.2]

□ hsa00920 Sulfur metabolism

55501 CHST12; carbohydrate (chondroitin 4) sulfotransferase 12

□ hsa00970 Aminoacyl-tRNA biosynthesis

1615 DARS; aspartyl-tRNA synthetase [EC:6.1.1.12] [SP:SYD_HUMAN]

□ hsa03022 Basal transcription factors

2965 GTF2H1; general transcription factor IIH, polypeptide 1, 62 kDa [SP:TFH1_HUMAN]

□ hsa03050 Proteasome

10213 PSMD14; proteasome (prosome, macropain) 26S subunit, non-ATPase, 14

□ hsa04010 MAPK signaling pathway

6416 MAP2K4; mitogen-activated protein kinase kinase 4 [EC:2.7.1.-] [SP:MPK4_HUMAN]

7850 IL1R2; interleukin 1 receptor, type II [SP:IL1S_HUMAN]

□ hsa04060 Cytokine-cytokine receptor interaction

1436 CSF1R; colony stimulating factor 1 receptor, formerly McDonough feline sarcoma viral (v-fms) oncogene homolog [EC:2.7.1.112] [SP:KFMS_HUMAN]

1524 CX3CR1; chemokine (C-X3-C motif) receptor 1 [SP:C3X1_HUMAN]

3556 IL1RAP; interleukin 1 receptor accessory protein

7850 IL1R2; interleukin 1 receptor, type II [SP:IL1S_HUMAN]

□ hsa04110 Cell cycle

1028 CDKN1C, cyclin-dependent kinase inhibitor 1C (p57, Kip2) [SP:CDNC_HIAN]

4171 MCM2; MCM2 minichromosome maintenance deficient 2, mitotin (S. cerevisiae)

4175 MCM6; MCM6 minichromosome maintenance deficient 6 (MIS5 homolog, S. pombe) (S. cerevisiae) [SP:MCM6_HUMAN]

5111 PCNA; proliferating cell nuclear antigen [SP:PCNA_HUMAN]

□ hsa04120 Ubiquitin mediated proteolysis 54926 UBE2R2; ubiquitin-conjugating enzyme E2R 2

□ hsa04210 Apoptosis

3556 IL1RAP; interleukin 1 receptor accessory protein

5573 PRKAR1A; protein kinase, cAMP-dependent, regulatory, type I, alpha (tissue specific extinguisher 1) [SP:KAP0_HUMAN]

□ hsa04310 Wnt signaling pathway

6934 TCF7L2; transcription factor 7-like 2 (T-cell specific, HMG-box)

□ hsa04350 TGF-beta signaling pathway

3398 ID2; inhibitor of DNA binding 2, dominant negative helix-loop-helix protein [SP:ID2_HUMAN]

□ hsa04610 Complement and coagulation cascades

712 C1QA; complement component 1, q subcomponent, alpha polypeptide [SP:C1QA_HUMAN]966 CD59; CD59 antigen p18-20 (antigen identified by monoclonal antibodies 16.3A5, EJ16, EJ30, EL32 and G344) [SP:CD59-HUMAN]

□ hsa04611

712 C1QA; complement component 1, q subcomponent, alpha polypeptide [SP:C1QA_HUMAN]

966 CD59; CD59 antigen p18-20 (antigen identified by monoclonal antibodies 16.3A5, EJ16, EJ30, EL32 and G344) [SP:CD59_HUMAN]

□ hsa04620 Toll-like receptor signaling pathway

6416 MAP2K4; mitogen-activated protein kinase kinase 4 [EC:2.7.1.-] [SP:MPK4_HUMAN]

6772 STAT1; signal-transducer and activator of transcription 1, 91 kDa [SP:STA1_HUMAN]

□ hsa04630 Jak-STAT signaling pathway

6772 STAT1; signal transducer and activator of transcription 1, 91 kDa [SP:STA 1_HUMAN]

868 CBLB; Cas-Br-M (murine) ecotropic retroviral transforming sequence b

□ hsa05110 Cholera—Infection

377 ARF3; ADP-ribosylation factor 3 [SP:ARF3_HUMAN]

A batch search of the Genetic Association database was performed for the following genes: CX3CR1, TRIM14, ARF3, BRD7, PILRB, ENTPD1, CSF1R, RABGAP1, ICAM2, KLHL2, PUM1, MTHFS, LY6E, MRPL47, NPM1, C12orf8, TNFAIP3, CHES1, SIP1, MYOZ2, ATP5J, IFI44, SEC14L1, G1P2, GTF2H1, FBXO2, USP18, ACPT, SP100, AIP, ABHD5, SCO2, PWWP1, RAN, GRN, MX1, SLC1A4, GZMB, SNRPA1, IMPDH1, TARDBP, ZCCHC2, IER5, CBLB, STAT1, WBSCR20A, MEA, TNRC6, MAK, TCF7L2, TINF2, HNRPH1, HNRPH2, GK, SART3, H1FX, PTP4A2, PSMD14, EIF3S4, BTN3A3, LETM1, TIMM23, HIVEP2, USP22, MT1L, C1QA, IL1RAP, MS4A7, NICAL, KBTBD7, C1orf29, PNUTL2, RPN2, ILF3, PCNA, HMGB1, BAG1, MCM2, TYMS, MT1X, CPD, COX15, MCM6, SN, C6orf133, BACE2, SYT6, OAS1, FACL2, OAS2, C6orf209, NUP98, PRKAR1A, OAS3, CHST12, FACL5, SLPI, CD59, IFIT1, IFI27, SORL1, RNPC4, IFIT4, HMGN4, CECR1, CDCA7, MTSS1, C6orf37, CDKN1C, RBPSUH, IL1R2, YWHAQ, RRM2, DARS, UBE2R2, SFRS7, FCGR2A, OASL, ID2, PLCL2, LGALS3BP, KPNA2, and MAP2K4.

Of these genes, the following hits were returned:

CX3CR1
1) Disease Class=Infection; Broad Phenotype (Disease)=HIV/SIV infection;
2) Disease Class=Unknown; Broad Phenotype (Disease)=Human Renal Transplantation;

SCO2
1) Disease Class=Cardiovascular; Broad Phenotype (Disease)=hypertrophic cardiomyopathy and cytochrome c oxidase deficiency;

FCGR2A
1) Disease Class=Infection; Broad Phenotype (Disease)=Severe Malaria;
2) Disease Class=Infection; Broad Phenotype (Disease)=fulminant meningococcal septic shock in children;
3) Disease Class=Immune; Broad Phenotype (Disease)=atopic disease;
4) Disease Class=Immune; Broad Phenotype (Disease)=rheumatoid arthritis;
5) Disease Class=Immune; Broad Phenotype (Disease)=systemic lupus erythematosus.

### Example 4

### Effects of two Globin mRNA Reduction Methods on Gene Expression Profiles from Whole Blood

Materials and Methods

Sample collection. With approval of the Lackland AFB IRB and after informed consent, approximately 25 ml of blood, filling 10 PAX tubes, were drawn from each healthy volunteer. Blood was drawn into PAX tubes by standard protocol {Preanalytix #23*}. All PAX tubes were maintained at room temperature for 2 hrs, then frozen at −20° C., stored at −80° C. for 5 days, and shipped on dry-ice to the Navy Research Laboratory in Washington, D.C. for processing.

Sample processing. Blood collection and RNA isolation was performed using the PAX System, which consists of an evacuated tube (PAX tube) for blood collection and a processing kit (PAX kit) for isolation of total RNA from whole blood {*Jurgensen #32; Jurgensen #33}. The isolated RNA underwent globin reduction procedures and was amplified, labeled, and interrogated on the HG-U133 plus 2.0 Genechip® microarrays (Affymetrix).

Total RNA isolation from blood. Frozen PAX tubes were thawed at room temperature for 2 hrs followed by total RNA isolation as described in the PAX kit handbook {*Preanalytix #24}, but modified to aid in tight pellet formation by increasing proteinase K from 40 μl to 80 μl (>600 mAU/ml) per sample, extending the 55° C. incubation time from 10 min to 30 min, and passing through a QIAshredder spin column (Qiagen). The optional on-column DNase digestion was not carried out. Purified total RNA was stored at −80° C.

Total RNA cleanup and concentration. For more complete removal of DNA from purified RNA, duplicate RNA samples were pooled, followed by in-solution DNase treatment using the DNA-free™ kit (Ambion), but without addition of DNase inactivation reagent. After DNase treatment, RNA were subjected to RNAeasy MinElute Cleanup (Qiagene cat#74204) and concentrated according to the manufacturer's procedure. Subsequently, one microliter from each sample was run on the bioanalyzer 2100 (Agilent) for assessment of RNA quality while the nanodrop (NanoDrop) was used for quantification. Usage of the bioanalyzer was analogous to capillary gel electrophoresis. This resulted in electropherograms displaying florescent intensity versus time, which correlates with the amount of RNA versus the size of RNA, respectively.

Globin reduction and target preparation. To remove globin mRNA, biotinylated globin capture oligos (Ambion Globinclear kit) and PNA (Affymetrix GeneChip Globin Reduction kit) were used according to modified manufacturers' procedures. In brief, for the Globinclear's procedure, biotinylated globin capture oligos were added to 5 μg total RNA and globin mRNA were removed by strepavidin magnetic beads. Then the remaining globin-reduced total RNA was purified using magnetic beads and eluted in 30 μL of water. One microliter of RNA was used for bioanalyzer measurement and the remaining RNA was concentrated to 8 μL using Speed Vac concentration at room temperature. For the PNA globin reduction procedure, 5 μg of total RNA in 9 μL BR5 from the RNAeasy MinElute Cleanup step was used for the downstream procedure. The column that came with the Globin Reduction kit was not used. All subsequent steps were as described in the GeneChip Expression Analysis Technical Manual version 701021 Rev. 3.

Database integration. Laboratory data contained information about the processing of samples from blood in PAX tubes to cRNA target preparation, as well as bioanalyzer and nanodrop measurements. Electropherograms were analyzed by the Biosizing software (Agilent) to output 28S/18S intensity ratios and RIN QC metrics while the nanodrop output RNA quantity and 260/280 ratios. Report files summarizing the quality of target detection for an array were generated by GeneChip® Operating Software 1.1 (Affymetrix). JMP (SAS) was used to join these various data tables together into a metadata table. For gene-expression data, Signal values were calculated using the Microarray Suite 5.0 algorithm with and without scaling to test the effects on various downstream analytical methods.

Statistical analysis. Statistical quality control and relations among metadata variables and gene expression profiles were analyzed in JMP. ANOVAs, multidimensional scaling, and functional analysis of gene-expression data were performed in Arraytools 3.2.0. Beta developed by Richard Simon and Amy Lam (http://Iinus.nci.nih.gov/BRB-ArrayTools.html). Heat-maps and dendrograms were graphed using dChip {Li, 2001 #41; Li, 2001 #42}. Scaled expression data showed no differences in Scale Factors among treatment groups.

Results

Quality of RNA, globin reduction, and target preparation. The following RNA samples were used to study the effects of two globin reduction methods on gene expression profiles:
1) Jurkat RNA isolated from Jurkat cell line (J)
2) Jurkat RNA with globin mRNA spiked-in (JG)
3) Paxgene RNA from whole blood (B)

The globin reduction protocols tested were:
1) Ambion's Globinclear method using biotinylated globin capture oligos (A)
2) Affymetrix' method using PNA oligos (P)
3) No globin reduction treatment as technical control (C).

The same lot of J and JG RNA were used throughout. RNA treated with Ambion globinclear had ˜90% recovery for J and JG RNA. The yields of cRNA for the Ambion group were the lowest among the three technical conditions for each RNA species; however, RNA purity judged by the ratio of 260/280 for Ambion globinclear group was the highest (Table 13).

TABLE 13
Comparison of pre-hybridization variables and post-hybridization chip results in RNA
species with different treatment

| | RNA | | | | |
|---|---|---|---|---|---|
| | Jurkat RNA | | | Jurkat RNA + Globin | |
| | Treatment | | | | |
| | Ambion | PNA | Control | Ambion | PNA |
| Starting | 4 | 4 | 4 | 4 | 4 |
| material (μg) | | | | | |
| Yields after | 3.56 ± 0.41 | 4 | 4 | 3.43 ± 0.24 | 4 |
| treatment | | | | | |
| Adjusted | 71.13 ± 5.412 | 96.4 ± 30.66 | 113.47 ± 40.77 | 58.33 ± 2.91 | 107.93 ± 29.99 |
| cRNA yield | | | | | |
| 260/280 for | 2.01 ± 0026 | 1.98 ± 0.035 | 1.92 ± 0.047 | 2.03 ± 0.02 | 1.95 ± 0.05 |
| cRNA | | | | | |

Results

| | | | | | |
|---|---|---|---|---|---|
| Present Calls | 46.8 ± 1.18 | 45.5 ± 0.62 | 44.8 ± 1.65 | 41.53 ± 0.83 | 37.4 ± 0.7 |
| (%) | | | | | |
| Scale Factors | 4.50 ± 1.38 | 3.98 ± 0.62 | 4.42 ± 0.52 | 5.13 ± 1.06 | 5.10 ± 0.50 |
| Background | 64.21 ± 12.46 | 68.47 ± 11.30 | 60.91 ± 3.71 | 56.06 ± 3.18 | 70.90 ± 5.86 |
| Noise | 3.36 ± 0.71 | 3.58 ± 0.70 | 3.40 ± 0.29 | 2.92 ± 0.28 | 4.02 ± 0.75 |
| 3′/5′ GAPDH | 1.06 ± 0.04 | 1.05 ± 0.03 | 1.09 ± 0.07 | 1.06 ± 0.07 | 1.09 ± 0.10 |
| 3′/5′ Actin | 1.33 ± 0.15 | 1.23 ± 0.06 | 1.31 ± 0.03 | 1.25 ± 0.01 | 1.17 ± 0.05 |

| | RNA | | | |
|---|---|---|---|---|
| | Jurkat RNA + | | | |
| | Globin | Paxgene | | |
| | Treatment | | | |
| | Control | Ambion | PNA | Control |
| Starting | 4 | 5 | 5 | 5 |
| material (μg) | | | | |
| Yields after | 4 | 3.71 ± 0.32 | 5 | 5 |
| treatment | | | | |
| Adjusted | 124.27 ± 30.96 | 25.87 ± 3.91 | 30.61 ± 17.05 | 41.18 ± 7.76 |
| cRNA yield | | | | |
| 260/280 for | 1.85 ± 0.02 | 2.13 ± 0.02 | 2.08 ± 0.02 | 2.06 ± 0.01 |
| cRNA | | | | |
| Results | | | | |
| Present Calls | 32.37 ± 1.56 | 39.33 ± 1.38 | 38.53 ± 2.39 | 32.77 ± 1.39 |
| (%) | | | | |
| Scale Factors | 5.41 ± 0.89 | 7.78 ± 1.82 | 7.40 ± 1.17 | 10.6 ± 80.71 |
| Background | 86.6 ± 4.22 | 57.59 ± 3.19 | 61.27 ± 5.58 | 54.27 ± 5.17 |
| Noise | 5.34 ± 0.10 | 3.23 ± 0.34 | 3.34 ± 0.45 | 3.07 ± 0.40 |
| 3′/5′ GAPDH | 1.14 ± 0.02 | 1.70 ± 0.11 | 3.59 ± 1.86 | 2.25 ± 0.11 |
| 3′/5′ Actin | 1.05 ± 0.03 | 2.55 ± 0.30 | 5.94 ± 3.74 | 3.16 ± 0.26 |

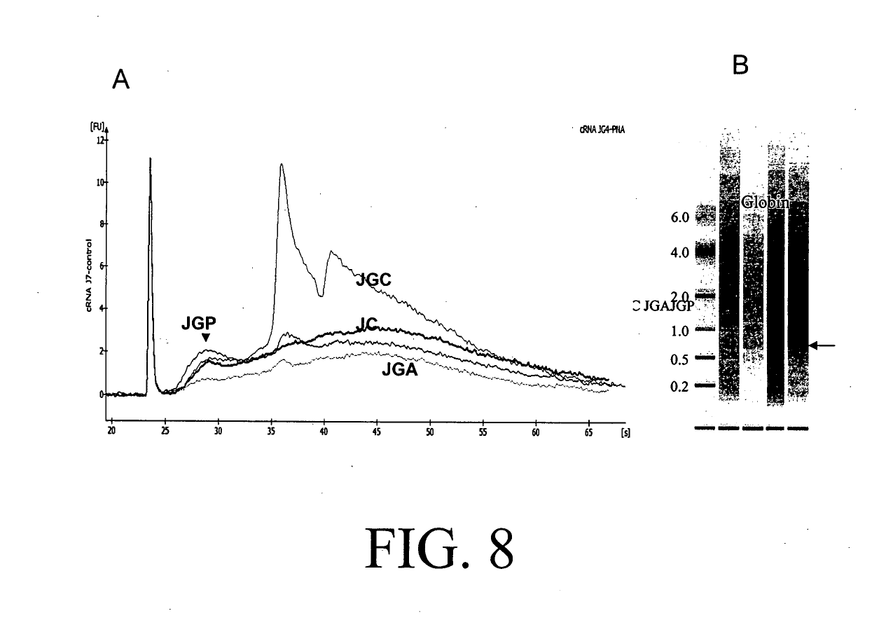

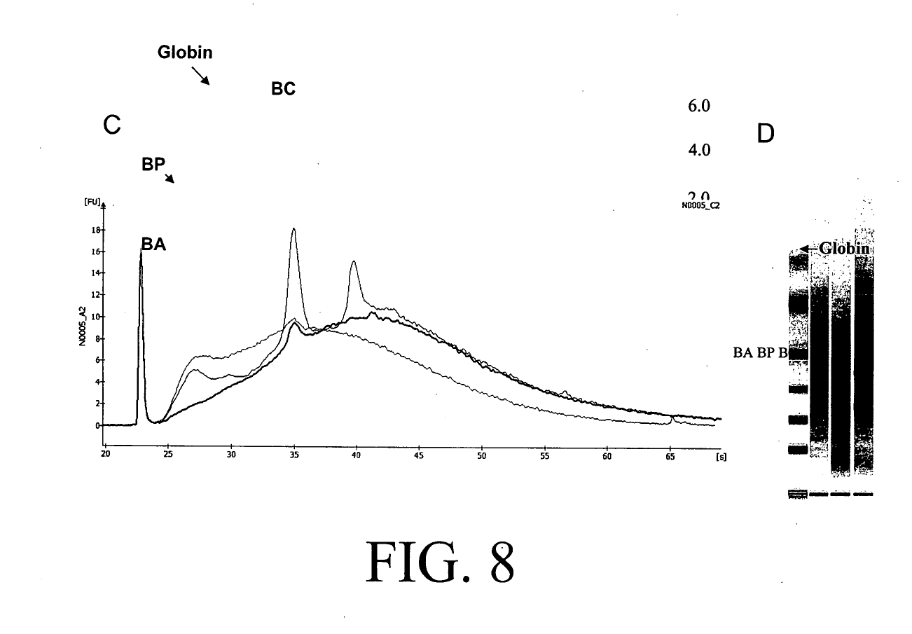

Profiles of cRNA for J and JG RNA compared using the bioanalyzer (FIG. 8A, B) indicated that JG RNA treated with Ambion (JGA) and JG RNA treated with PNA (JGP) had a significantly reduced globin peak (arrow in FIG. 8A) and globin band (FIG. 8B) relative to JGC. The electropherogram and gel profiles for JGA and JGP were very similar to Jurkat RNA without treatment (JC). There was no difference in cRNA profiles derived from JC, or Jurkat RNA treated with Ambion globinclear (JA) or with the PNA globin reduction procedure (JP) (data not shown).

There was no biological variation among paxgene RNA, since paxgene RNA used for each technical condition was derived from the pooled paxgene tubes collected from the same individual in one bleeding. Paxgene RNA with a ratio 260/280 between 1.9-2.1 was used as starting RNA and ˜75% recovery for paxgene RNA (Table 13)

Decreasing globin peaks and band were also seen in cRNA profiles derived from paxgene RNA samples treated with Ambion globinclear (BA) and PNA globin reduction (BP) compared to BC (no treatment) (arrow in FIGS. 8C and D). However, the cRNA size from BA was larger than BP. Overall, our result demonstrated that both Ambion globinclear and the PNA globin reduction protocols decreased globin mRNA contaminants effectively.

Quality of microarray measurements for each technical condition For microarray data quality assessment, poly A control graphs for each microarray were plotted using scaling signal intensity and non-scaling data. Linearity was achieved among the four control probe sets for all samples (data not shown). All of the constants and major variables, such as scale factors (SF), background, and noise (see Table 13) obtained from RPT report were assessed using the ANOVA and Wilcoxon tests. There was no statistically significant difference in SF and noise among JA, JC, JP, JGA, JGP and JGC, neither in BA, BP and BC. Thus, scaling signal intensities for all probe sets were used in the gene expression profile comparison. For Jurkat RNA, background was highest in JGC and was significantly different from the others, possibly due to the spiked globin mRNA. There was no difference in background among all paxgene RNA. Ratios of 3′/5′ GAPDH for all microarrays were all below 5 and indicated that there was no RNA degradation. A slightly higher ratio of 3′/5′ Actin and GAPDH was noted in paxgene RNA with PNA treatment, possibly due to the reduction of cRNA size (BP in FIG. 8C). Since no significant difference in other variables was detected, we conducted further statistical analysis and comparison of gene expression profiles.

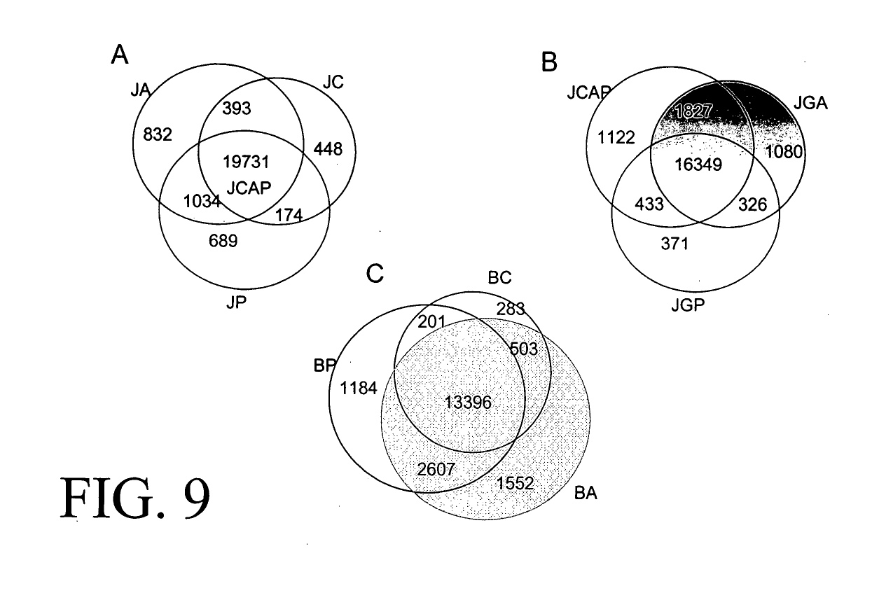

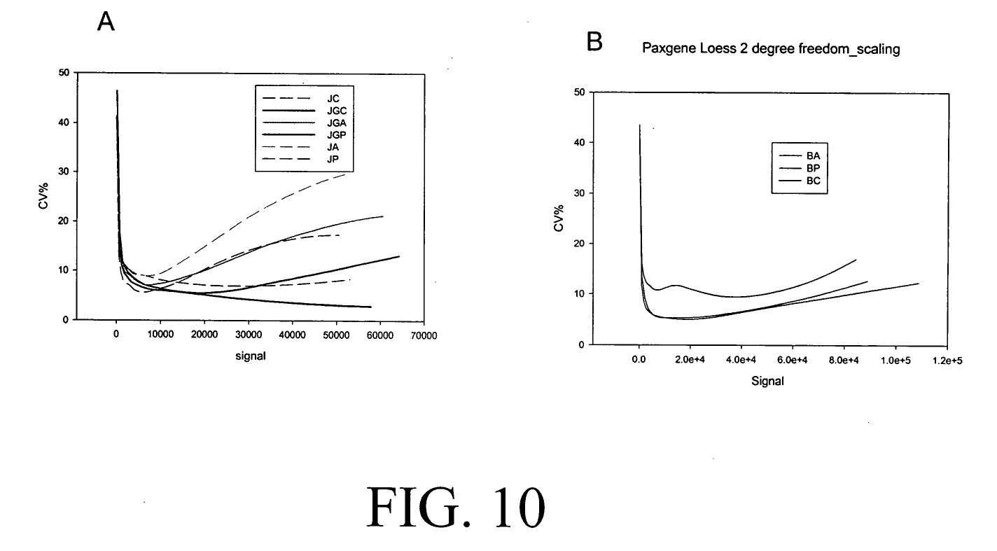

Globin removal increases number of present calls (%) and call concordance in gene expression Removal of globin by both methods significantly increased the number of present calls (%) in JGA, JGP, BA, BP compared to their corresponding controls, JGC and BC (ANOVA, Wilcoxon test); however, there was no difference among three technical conditions in Jurkat RNA using the ANOVA and Wilcoxon tests. Further analysis of these methods with the student t-test revealed statistically significant higher present calls in JGA than JGP (student t-test, p<0.05), but there was no significant difference in paxgene RNA between BA and BP (Table 13). The present call concordance among Jurkat RNA for the three technical conditions was compared and a gene subset containing 19731 genes, called JCAP, which was not affected by technical conditions (JCAP in FIG. 9A) was identified to serve as a control gene set for JG RNA. The present calls for JGA and JGP were then compared to JCAP, resulting in 18176 (=16349+1827) genes present in both JCAP and JGA and 16782 (=16349+433) genes present in both JCAP and JGP (FIG. 9B), while there were only 14069 genes present in both JCAP and JGC (data not shown). Our data indicated that JGA exhibited 1394 additional concordant calls relative to JGP and 4107 additional concordant calls relative to JGC. For the paxgene RNA, BA/BP had 2104 additional concordant calls present relative to BA/BC and 2406 additional concordant calls present relative to BC/BP (FIG. 9C).

In addition to assessing present call concordance, the overall call concordance excluding margin calls between Jurkat and JG RNA was tabulated and the percentages of false positive and negative among technical conditions were compared (Table 14). Our data demonstrated that JGA and JGP increased concordant present calls by 8% and 5%, respectively, relative to JGC had 7% and 4% increased false negative calls compared to JGA and JGP, respectively. False positive present calls occurred in 1% and 0.22% of JGA and JGP processed samples, respectively, compared to JGC. Calculated sensitivities for JGA, JGP and JGC compared to the “gold standard” of Jurkat RNA were 86%, 79.5% and 68.2%, respectively. Specificity was retained with all processing methods with specific values for JGA, JGP and JGC being 94.3%, 96.2% and 96.2%, respectively. The data suggests that the Ambion globinclear method had significantly higher sensitivity percent present calls without significant loss of specificity relative to JGC (Table 15).

TABLE 14
Comparison of Pearson correlation coefficient

| | Pearson correlation coefficient |
|---|---|
| Treatment Description | Mean ± stdev |
| Triplates in each sample | |
| Jurkat-Ambion | 0.985 ± 0.009 |
| Jurkat-PNA | 0.993 ± 0.003 |
| Jurkat-no treatment | 0.993 ± 0.001 |
| Jurkat + Globin-Ambion | 0.992 ± 0.005 |
| Jurkat + Globin-PNA | 0.996 ± 0.001 |
| Jurkat + Globin-No treatment | 0.993 ± 0.004 |
| Paxgene-Ambion | 0.997 ± 0.001 |
| Paxgene-PNA | 0.987 ± 0.009 |
| Paxgene-No treatment | 0.996 ± 0.001 |
| Between Techniques | |
| Jurkat RNA | |
| Ambion vs. No treatment | 0.986 ± 0.005 |
| PNA vs. No treatment | 0.992 ± 0.004 |
| Ambion vs. PNA | 0.987 ± 0.006 |
| Jurkat + Globin-RNA | |
| Jurkat RNA | 0.966 ± 0.011 |
| Ambion vs. No treatment | 0.983 ± 0.003 |
| PNA vs. No treatment | 0.985 ± 0.002 |
| Paxgene blood RNA | |
| Jurkat RNA | 0.978 ± 0.006 |
| Ambion vs. No treatment | 0.967 ± 0.006 |
| PNA vs. No treatment | 0.979 ± 0.003 |
| Between RNA species | |
| Jurkat vs. Jurkat + Globin | |
| JA/JGA | 0.962 ± 0.007 |
| JP/JGA | 0.963 ± 0.006 |
| JC/JGA | 0.963 ± 0.003 |
| JA/JGP | 0.960 ± 0.006 |
| JP/JGP | 0.967 ± 0.005 |
| JC/JGP | 0.967 ± 0.002 |
| JA/JGC | 0.942 ± 0.015 |
| JP/JGC | 0.946 ± 0.014 |
| JC/JGC | 0.952 ± 0.010 |

TABLE 15
Cross tabulation for call concordance

| | | JGA | | JGP | | JGC | |
|---|---|---|---|---|---|---|---|
| | Calls (%) | P | A | P | A | P | A |
| Jurkat RNA | P | 21100 ± 367 | 3455 ± 594 | 19350 ± 338 | 5055 ± 761 | 16733 ± 679 | 7583 ± 1003 |
| Jurkat RNA | A | 1359 ± 261 | 27296 ± 568 | 938 ± 165 | 27795 ± 714 | 822 ± 124 | 27926 ± 740 |

P = PNA globin reduction
A = Ambion globinclear

Variance caused by two globin reduction methods Signal variation among triplicates was assessed by comparing the coefficient of variance (CV) (FIG. 10). Since there was no statistical difference in scaling factors for each technical condition, scaling signal intensities for all probe sets were used to plot CV graphs and Loess fitting with 2 degree freedom was introduced to fit the curves. Higher CV introduced by technical conditions was seen either in JA or JP compared to JC (dash lines in FIG. 10A). However, globin removal by biotinylated globin oligos and PNA significantly reduced the variation for each corresponded technical condition in JG RNA (solid lines in FIG. 10A). JA had the highest CV among all, especially in gene sets with signal intensities greater than 10⁴. This high CV could be due to the multistep globinclear procedure. In contrast, in paxgene RNA, CV among globinclear triplicates was as low as no treatment. RNA species and purity may affect technical variation caused by globinclear. In paxgene RNA, CV for PNA triplicates was the highest among all technical conditions (FIG. 10B) possibly due to reduction of cRNA size from PNA oligo treatment (FIG. 8C).

In addition to CV(%) comparison, Pearson correlation coefficient(again—it was difficult for me to determine whether any of these observations was significant) was also calculated and compared in each triplicate between technical conditions within the same RNA species and between RNA species (Table 15). Higher signal correlation was seen within triplicates compared to that seen between technical conditions or between RNA species. In JG RNA, globin removal by biotinylated globin oligos (Ambion) had lower signal correlation with no treatment JGC (0.966), but JGP has higher correlation (0.983) with JGC. This indicated that globinclear JG RNA has more difference in gene expression profile relative to JGC than JGP. In paxgene RNA, PNA treatment has lower signal correlation (0.967) with no treatment (BC), but JGA higher correlation (0.978) with BC. This suggested that more difference in gene expression were seen in BP and BC than BA and BC. Removal of globin mRNA from paxgene RNA or JG RNA resulted in higher signal correlation in the same RNA species or between Jurkat and Jurkat+Globin RNA (between RNA species in Table 15).

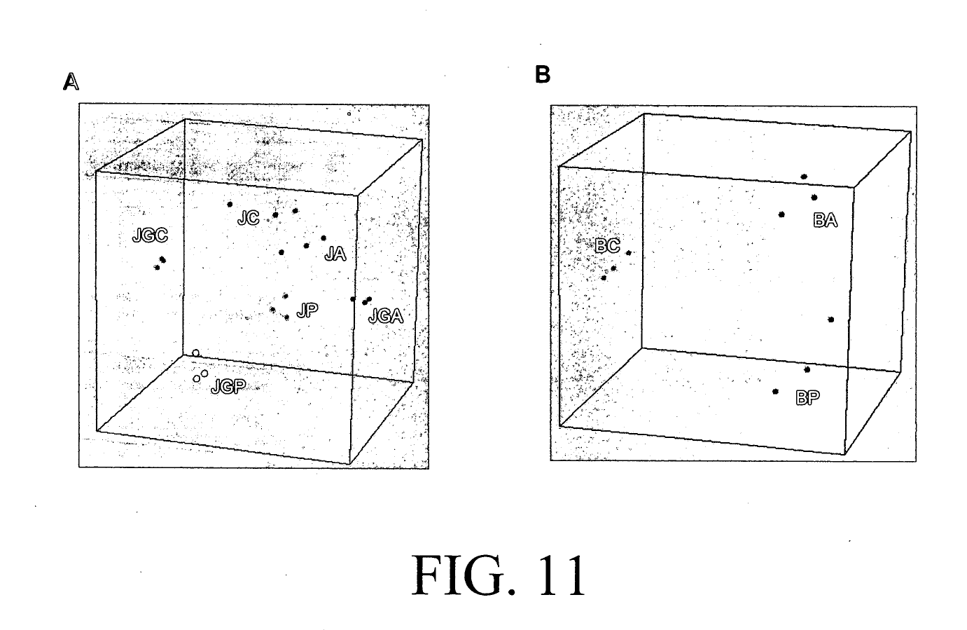

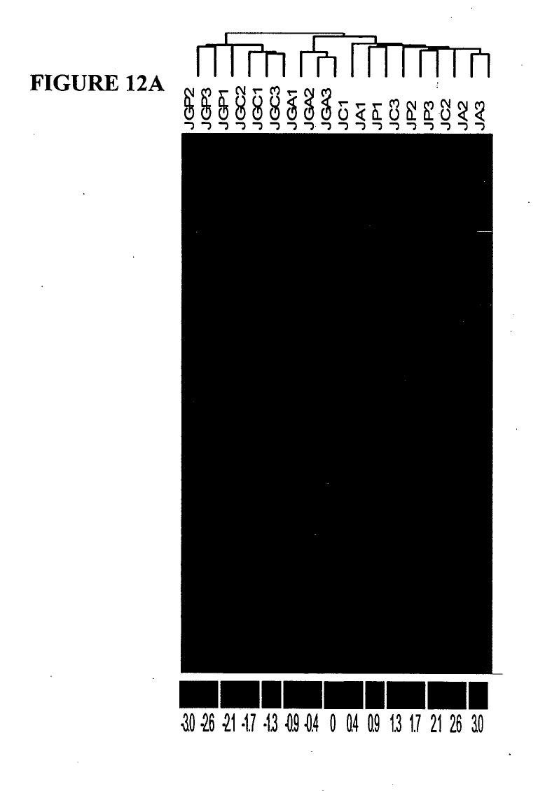

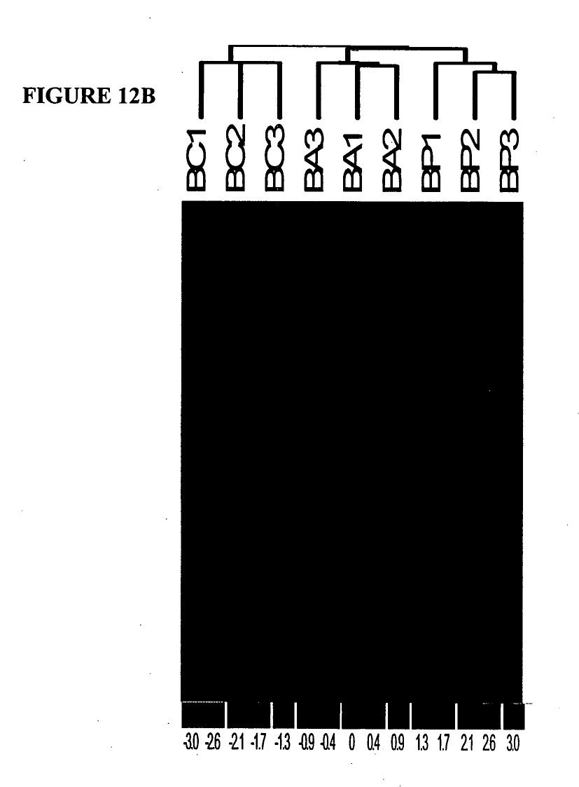

Multidimensional scaling cluster analysis of gene expression profiles To further evaluate correlation between groups of samples for each technical condition, multidimensional scaling (MDS) cluster analysis was conducted. Since non-scaling data and scaling data exhibited similar clustering pattern, we only showed MDS plots using all probe sets with non-scaling signal intensities (FIG. 11). Our data indicated that each triplicate was tightly clustered and triplicate clusters for Jurkat RNA with different technical conditions were close to one another. Triplicate clusters for JG RNA with different technical conditions were more separated from each other than those from Jurkat RNA with the JGA triplicate cluster located closest to the Jurkat RNA cluster (FIG. 11A). Paxgene RNA also formed three separate triplicate clusters corresponding to each technical condition (FIG. 11B).

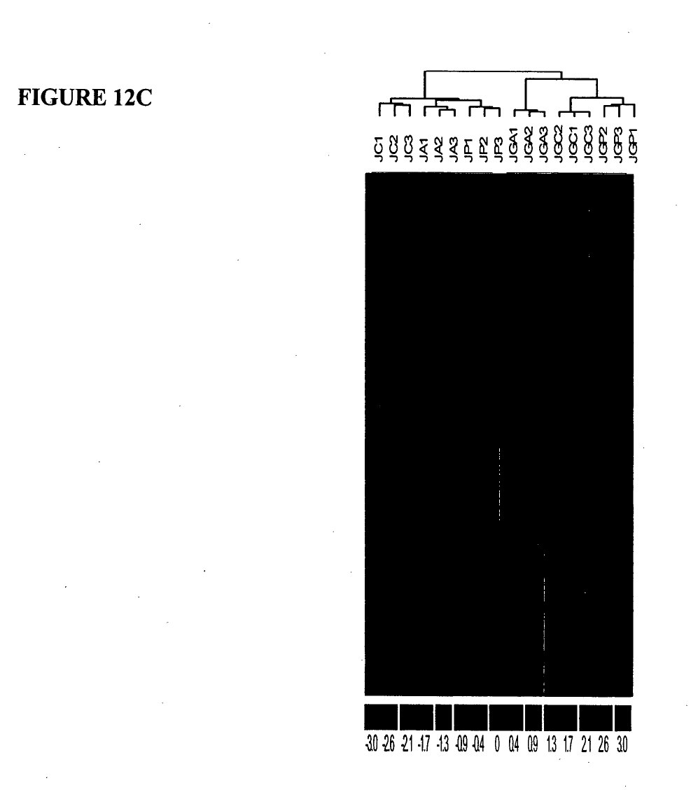

Hierarchal cluster analysis of gene expression profiles The overall expression profiles for Jurkat and JG RNA samples with different technical conditions were analyzed using center correlation and average linkage parameters (FIG. 12A). Consistent with the MDS plot, removal of globin mRNA from JG RNA samples by biotinylated globin oligos revealed similar gene expression profiles to the Jurkat RNA group and were clustered in the same group with Jurkat RNA samples (FIG. 12A). These 18 chips were grouped into six classes as JA, JP, JC, JGA, JGP and JGC and gene expression profiles were compared among these classes using the univariate test in the Random Variance model. The class comparison resulted in 8614 differentially expressed genes, which were further clustered using dChip software analysis.

We divided these differentially expressed genes into 4 groups as indicated on the right side of the dendrogram (FIG. 12B). Group I represented most of down-regulated genes in JGA and all Jurkat RNA samples and it included globin genes and genes affected by globin mRNA cross hybridization. Group II represented upregulated genes in Jurkat RNA samples, but down-regulated in all of JG samples. This could include some false negative genes shown in Table 15. False negative genes could result from a negative impact caused by globin RNA noise resulting in low signal intensities Group III represented genes that could be revealed after globin RNA reduction with biotinylated globin oligos protocol, but remained down-regulated with PNA protocol and no treatment (III in FIG. 12B). Group IV represented unique up-regulated genes resulting from biotinylated globin oligos protocol. This group could include some false positive genes in Table 14.

Using the same approach, gene expression profiles and differentially expressed gene profiles among BA, BP, and BC, with total of 9 paxgene blood RNA samples were analyzed and clustered using center correlation and average linkage. Our results revealed that removal of globin mRNA using biotinylated globin oligos and PNA oligos revealed more similar gene expression profile and were clustered within the same group possibly due to globin reduction (FIG. 12C). Moreover, there were 1988 differentially expressed genes among paxgene blood RNA samples using the univariate test for Random Variance model (FIG. 12D). The cluster analysis result indicated that differentially expressed gene profiles for BA and BC were more similar than BP. This is consistent with higher correlation between BA and BC (Table 14).

### Example 5

### Surveillance of Transcriptomes in Basic Military Trainees with Normal, Febrile Respiratory Illness, and Convalescent Phenotypes

Materials and Methods

Entry criteria and sample collection. LAFB is the location of Basic Military Training for all recruits to the United States Air Force. The BMTs are organized into flights of 50-60 individuals that eat, sleep, and train in close quarters. As many as 40-50 BMTs/week present with FRI and 50-70% are due to adenovirus. With approval of LAFB IRB and after informed consent, approximately 15 ml of blood, filling 4 to 5 PAX tubes, were drawn from each volunteer. On day 1-3 of training, blood was drawn from healthy BMTs into PAX tubes by standard protocol {Preanalytix #23}, but no nasal wash was collected for this group. During training, BMTs who presented with a temperature of 38.1° C. or greater and FRI provided a nasal wash and blood draw. These individuals were categorized into either the FRI without adenovirus or with adenovirus group. Approximately three weeks after sample collection from the FRI volunteers with adenovirus, additional blood and nasal wash were collected to constitute samples for the convalescent group. All PAX tubes were maintained at room temperature for 2 hrs, then frozen at −20° C. and shipped on dry-ice to the Navy Research Laboratory in Washington, D.C. for processing. Nasal washes were performed using a standard protocol, with 5 ml of normal saline lavage of the nasopharynx, followed by collection of the eluent in a sterile container. Nasal wash eluent was stored at 4° C. for 1-24 hrs before being aliquotted and sent for adenoviral culture. All BMTs underwent standardized questionnaires before each sample collection. Healthy individuals were screened for acute medical illness within 4 weeks of arriving at basic training. BMTs were screened for race/ethnicity, allergies, recent injuries, and smoking history to assess confounding variables for gene expression. The duration and type of respiratory symptoms to include sore throat, sinus congestion, cough, fever, chills, nausea, vomiting, diarrhea, fatigue, body aches, runny nose, headache, chest pain and rash were recorded. A physical examination was recorded.

Sample processing. Blood collection and RNA isolation was performed using the PAX System, which consists of an evacuated tube (PAX tube) for blood collection and a processing kit (PAX kit) for isolation of total RNA from whole blood {Jurgensen #32; Jurgensen #33}. The isolated RNA was amplified, labeled, and interrogated on the HG-U133A and HG-U133B Genechip® microarrays (Affymetrix), noted here as A and B arrays, respectively.

Total RNA isolation from blood. Frozen PAX tubes were thawed at room temperature for 2 hrs followed by total RNA isolation as described in the PAX kit handbook {Preanalytix #24}, but modified to aid in tight pellet formation by increasing proteinase K from 40 μl to 80 μl (>600 mAU/ml) per sample, extending the 55° C. incubation time from 10 min to 30 min, and the centrifugation time to 30 min or more. The optional on-column DNase digestion was not carried out. Purified total RNA was stored at −80° C.

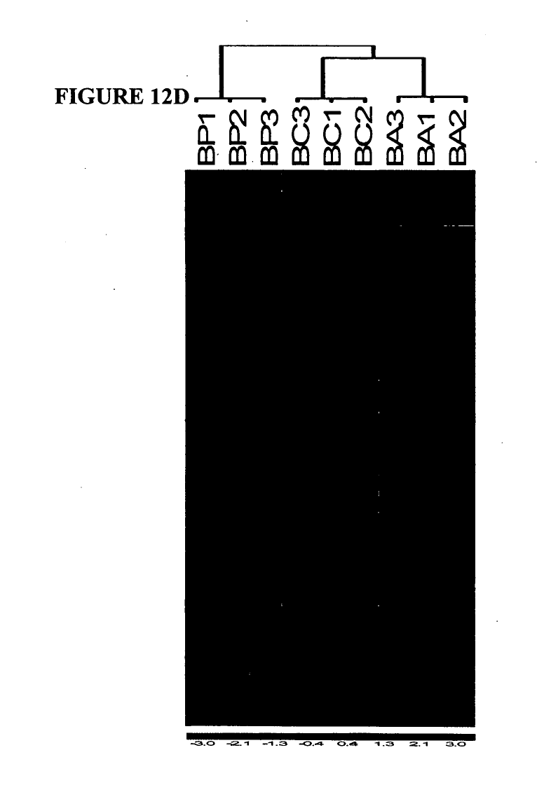

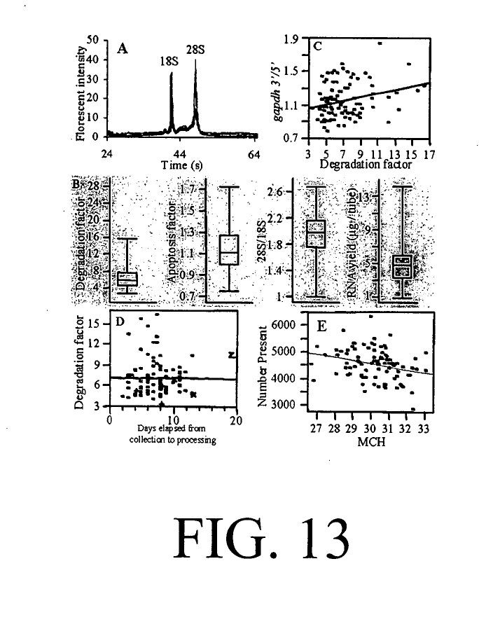

Target preparation. For more complete removal of DNA from purified RNA, duplicate RNA samples were pooled, followed by in-solution DNase treatment using the DNA-free™ kit (Ambion). However, to facilitate removal of the DNase inactivating beads, the completed reaction was spun through a spin column (Qiagen, Cat#79523), rather than attempting to pipette off the supernatant without disturbing the bead pellet. Subsequently, one microliter from each sample was run on the bioanalyzer (Agilent) for assessment of RNA quality and quantity. The usage of the bioanalyzer was analogous to capillary gel electrophoresis. This resulted in electropherograms displaying florescent intensity versus time (FIG. 13a), which correlates with the amount of RNA versus the size of RNA, respectively. Next, 5 μg of RNA were concentrated via ethanol precipitation as previously described {Thach, 2003 #18}. All subsequent steps were as described in the GeneChip Expression Analysis Technical Manual version 701021 Rev. 3.

Database integration. The database consisted of clinical data such as information transcribed from standardized questionnaires, the complete blood count (CBC), and the handling of blood samples. Laboratory data contained information about the processing of samples, from blood in PAX tubes to RNA extraction, as well as subsequent bioanalyzer measurements. Electropherograms were analyzed by the Biosizing (Agilent) software to output 28S/18S intensity ratios and RNA yields, and by the Degradometer 1.1 {Auer, 2003 #26} software to consolidate, scale, and calculate degradation and apoptosis factors. Report files summarizing the quality of target detection for an array were generated by GeneChip® Operating Software 1.1 (Affymetrix). JMP (SAS) was used to join these various data tables together into a metadata table with more than a thousand columns. For gene-expression data, Signal values were calculated using the Microarray Suite 5.0 algorithm with no scaling or normalization. This allows for subsequent testing of various scaling and normalization methods.

Statistical analysis. Statistical quality control and relations among metadata variables were analyzed in JMP. ANOVAs and class prediction of phenotypes using gene-expression data were performed in Arraytools 3.2.0 Beta developed by Richard Simon and Amy Lam (http://linus.nci.nih.gov/BRB-ArrayTools.html). Heat-maps and dendrograms were graphed using dChip {Li, 2001 #41; Li, 2001 #42}. Analysis of gene functions was aided by Arraytools and EASE {Hosack, 2003 #30}. Data analysis was performed primarily by D.T.

Scaling was carried out for gene-expression data. For each blood sample, the same hybridization cocktail went onto the A and then the B array, allowing concatenation of the data from the two arrays to form a virtual array. This bypassed issues with analyzing the two data sets separately. The 100 control probesets common between the A and B arrays were selected based on stability in expression from a large study of various tissue types {Affymetrix, 2002 #27}. Thus, all array data were scaled to a target value of 500 using the trimmed mean of the 100 control probesets. This resulted in stable Scale Factors (SF) over time and no differences in SF among the infection status phenotypes (ANOVA, P.=0.1047 A arrays, P=0.1782 B arrays). This scaling method allowed for the concatenation of corresponding A and B arrays and should also remove variations that are not gene-specific.

Results

Clinical Phenotypes. Thirty healthy, 19 with FRI and negative by culture for adenovirus, 30 with FRI and positive by culture for adenovirus, and 30 convalescing from adenovirus-positive FRI were enrolled in this study. Enrollees in these four infection status phenotypes were matched for age±3 years and race/ethnicity. Only male BMTs were enrolled. After selection of samples meeting standards for gene expression analysis, 17 FRI without adenovirus had been ill for 5±3 days (median±SD), whereas 26 FRI with adenovirus had been ill for 8±4 days (P=0.006, Wilcoxon). The incidence of symptoms over all the groups was: sore throat (95.3%), cough (93%), sinus congestion (90.7%), headache (88%), chills (84%), rhinorrhea (81%), body aches (65%), malaise (63%), nausea (54%), diarrhea (14%), pleuritic chest pain (14%), vomiting (14%), and rash (0%), with no significant differences between the FRI groups. There was also no significant difference in allergies, recent injuries, and smoking history among the infection status phenotypes.

Quality and variations of RNA derived from PAX system from the BMT population. In order to identify clinically relevant gene expression profile differences for phenotypes in a population, it is essential that the RNA sample applied to the microarray is representative of the amount of transcripts in vivo. The PAX system was used to minimize handling of blood cells post collection and to immediately stabilize RNA and halt transcription. We previously have shown two methods using this PAX system that provide stable RNA for microarray analysis {Thach, 2003 #18}.

To assess RNA quality on each of the 95 microarrays analyzed in this study, recently published metrics derived from electropherograms of the RNA were used {Auer, 2003 #26}. Assessment of the degradation factor, which is the ratio of the average intensity of bands of lesser molecular weight than the 18S ribosomal peak to the 18S band intensity multiplied by 100, demonstrated minimal degradation of RNA (FIG. 13). This degradation factor for the samples correlated with gapdh 3′/5′ on the A arrays (FIG. 13c; r=0.3, P=0.008, ANOVA) and actin 3′/5′ on the B arrays (r=0.2; P<0.05, ANOVA), the internal measurements for assessment of RNA quality on the microarray. There was no significant correlation between 28S/18S versus degradation factor, gapdh 3′/5′, and actin 3′/5′, suggesting that the degradation factor is a superior method for assessing RNA quality for microarray analysis. No significant difference in degradation factor was seen among the phenotype groups.

Assessment of the apoptosis factor, which is the ratio of the height of the 28S to 18S peak {Auer, 2003 #26}, suggested that a high percentage of blood cells underwent apoptotic cell death. The distribution of the degradation factor, apoptosis factor, 28S/18S, and yields of total RNA are shown in FIG. 13b. No significant difference in apoptosis factor was seen among the phenotype groups. There was no significant correlation between duration of freezing and degradation factor (FIG. 13d); nor was there correlation with apoptosis factor, RNA yield, 28S/18S, or gapdh and actin 3′/5′.

We determined if blood cell type heterogeneity affected the sensitivity of transcript detection. Assessment of complete blood count (CBC) variables that affect the number of present calls on the microarray demonstrated a linear correlation between number of probesets called Present and Mean Corpuscular Hemoglobin (MCH). A significant effect was detected (r=0.272; P=0.008, ANOVA) for the B arrays only (FIG. 13e). The equation of the regression line suggested that for every picogram increase in hemoglobin, there is a loss in present detection calls of 100 probesets or 2% of the average number of present called probesets on the B arrays. There was no difference in MCH among the infection status phenotypes.

Quality of microarray measurements of PAX system-derived RNA from the BMT population. Individual control charts versus the date of microarray scanning were plotted to look for stability of quality metrics over time, determine outliers, and compare with values proposed by the array manufacturer. The percent Present of transcripts was 32±10 (average±3SD) for A arrays and 21±6 for B arrays. The gapdh and actin 3′/5′ values were less than three, the upper-limit proposed by Affymetrix {Affymetrix, 2004 #29}. Noise was 3.6±1.3 for A arrays and 2.9±0.8 for B arrays. Average Background was 100±48 for A arrays and 78±33 for B arrays. After exclusions of array sets that were known to have been processed differently or erroneously, a total of 95 A and B array sets with stable quality metrics remained. These 95 sets were processed in batches with nearly equal representation of the four infection status phenotypes. Therefore, comparisons among these four groups should detect biological differences as these groups have similar variations due to processing.

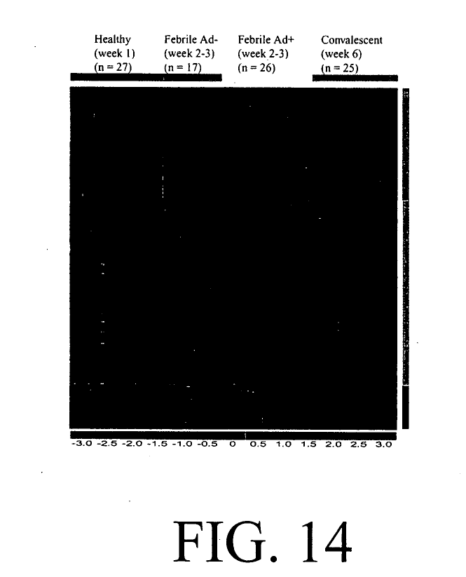

Gene expression profiles. The gene expression profiles were displayed on a heat-map with hierarchical clustering of transcripts to characterize and visualize patterns in the profiles of our cohort (FIG. 14). Initial examination revealed a large number of transcripts with high expression levels (FIG. 14, orange bar) and a smaller number of transcripts with low expression levels (FIG. 14, purple bar) in the febrile group compared to the non-febrile healthy and convalescent patients. There were also transcripts that showed differences between healthy and convalescent patients (FIG. 14, gray bar), while there was no obvious group of transcripts that showed differences between febrile without adenovirus versus febrile with adenovirus from this visual inspection. Within each group, inter-individual variation was observed, suggesting diverse immune responses in this population.

Class prediction of infection status phenotype. The pattern recognition above suggested that there were transcripts with differences in expression levels among healthy, febrile, and recovered patients. Therefore, class prediction was performed, to find sets of transcripts that best classify the four infection status phenotypes. Probesets with >80% absent calls across samples were filtered resulting in 15,721 probesets for further analysis. For supervised class prediction, the class labels for the febrile group were determine from respiratory viral culture results identifying presence or absence of adenovirus.

FIG. 14 suggested that the fever status of individuals was the predominant source of variation in gene expression profiles among samples and this was confirmed by unsupervised clustering of samples. Thus, supervised class prediction analysis was used to find sets of transcripts that classified non-febrile versus febrile patients first (node 1), then of the non-febrile patients, further classified to healthy or convalescent (node 2), and among the febrile patients, further classified to without or with adenovirus infection (node 3). The segregation of the samples via this nodal scheme was confirmed via binary tree class prediction analysis.

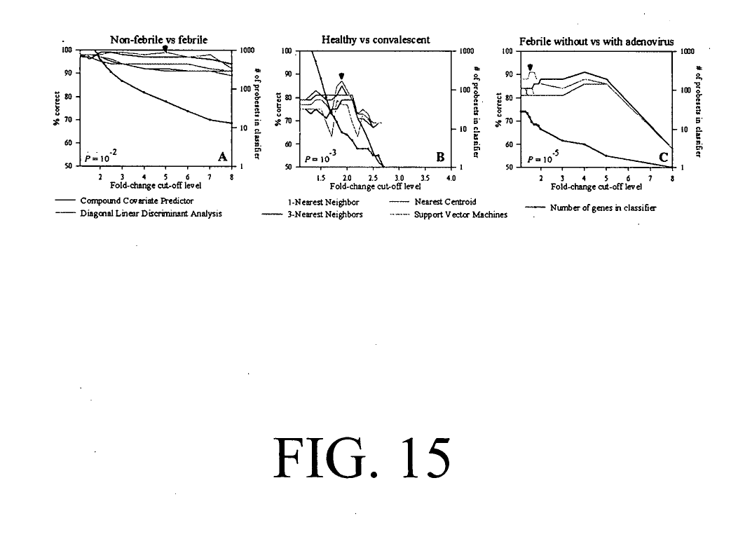

Unlike data from cancer studies {Golub, 2004 #34; Valk, 2004 #9}, there are no reported transcript selection methods or class prediction algorithms that are optimal for classification of infectious diseases. Therefore, we determined the transcript selection method and classification algorithm that would result in the highest percent correct classification during leave-one-out cross-validation. To estimate the optimal transcript selection parameters for classification in each node, the cut-off level of the univariate P-value was varied, selecting for probesets that showed statistically significant differences between the two groups at a P-value that was equaled to or smaller than a set cut-off level. As the P-value cut-offs became more stringent, the number of probesets selected decreased. For each P-value cut-off level, the selected probesets were subsequently used to classify the samples using various algorithms along with cross-validation analysis. For classification of node 1, 2 and 3, an optimal P-value cut-off level of 10⁻², 10⁻³, 10⁻⁵ (FIG. 15a-c, lower-left corner) was chosen, respectively.

Once an optimal P-value cut-off level was estimated and held constant, the additional criterion of fold-change cut-off threshold was varied (FIG. 15a-c, x-axes) for each node. FIG. 15 shows the percent-correct traces for the six algorithms tested tracking closely as fold-change cut-off level increases, but can differ by as much as 10−20% between methods. The black arrows in FIG. 15 indicate an optimal percent-correct classification at the specific P-value and fold change cut-off. For non-febrile vs. febrile, a percent correct call of 99% was achieved using the support vector machines algorithm at a P-value cut-off level of 10⁻² and a fold-change threshold of >5 which selected for 47 probesets to be in the classifier (FIG. 15a). For classification of healthy versus convalescent patients, an optimal percent correct of 87% using the diagonal linear discriminant analysis algorithm at a P-value cut-off level of 10⁻³ and a fold-change threshold of >1.9 which selected for 8 probesets to be-in the classifier was obtained (FIG. 15b). For classification of febrile patients without- versus with adenovirus infection, an optimal percent-correct of 91% using the support vector machine algorithm at a P-value cut-off level of 10⁻⁵ and a fold-change threshold of >1.7 which selected for II probesets to be in the classifier was obtained (FIG. 15c).

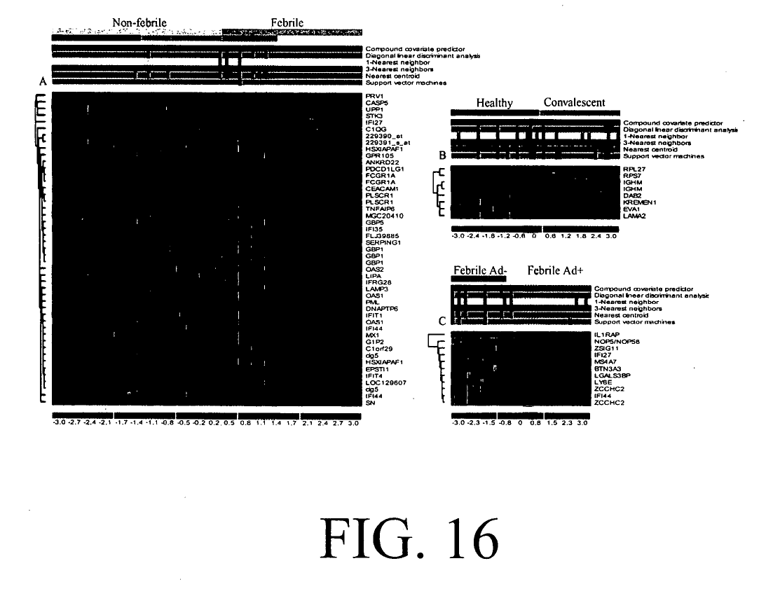

The samples that were misclassified by various algorithms and the associated gene expression profiles for the selected transcript set are shown in FIG. 16. For node 1, no individuals were misclassified in the febrile with adenovirus group and misclassified samples tended to belong to the febrile without adenovirus or the convalescent group. For node 2, the misclassified samples seemed to be equally distributed between healthy and convalescent, while for node 3, the misclassified samples tended to be in the febrile without adenovirus group. One observes that some samples were misclassified regardless of algorithm.

The estimated optimal percent-correct classification of non-febrile versus febrile, healthy versus convalescents, and febrile without versus with adenovirus infection patients were 99%, 87%, and 91%, respectively. To determine the reliability of these percentages, the permutation test was performed with 2000 permutations. This resulted in P-values of <0.0005, 0.001, and <0.0005, respectively.

Functions of genes in the classifier sets. The identifiers of the discovered transcript sets for the class prediction results are shown in FIG. 16. The 47 probesets used to classify fever status (FIG. 16a and Table 7) represent 40 transcripts. These included many that are induced by interferon, including: IFI27, IFI44, IFI35, IFRG28, IF1T1, IF1T4, OAS1 , OAS2, GBP1, CASP5, MX1, and G1P2. Furthermore, OAS1 and OAS2 catalyze 2′, 5′ oligomers of adenosine to activate RNaseL and inhibit cellular protein synthesis, while MX1 is a member of the GTPase family. OAS1, OAS2, and MX1 have been shown to have antiviral functions, and interestingly, have also been found to be activated shortly after infection of nonhuman primates with high titers of smallpox {Rubins, 2004 #35}. Transcripts involved in the complement cascade, C1QG which is downstream of antibody/antigen complexes and SERPING1 which inhibits activation of the first component of complement were associated with fever. The TNF-alpha and IL-1 induced gene, TNFAIP6, which is a secretory protein involved in extracellular matrix stability and cell migration, and STK3 and CASP5, which are involved in the MAPK signaling pathway and are downstream of the TNF and IL1 receptors were identified as class predictors. FCGR1A, which functions in the adaptive immune response and binds IgG, was part of the classifier. Other transcripts with associated known functions less clearly related to FRI or with unknown functions were also identified. Some gene ontology descriptions and, in parenthesis, their ratios of observed to expected number of occurrences were as follows (see Tables 8-9): GTP binding (6), guanyl nucleotide binding (6), response to virus (32), immune response (8), defense response (7), response to pest/pathogen/parasite (6), and response to stress (3).

The 8 probeset classifier (Table 10) for distinguishing healthy versus convalescent patients mapped to 7 transcripts, including RP127 and RPS7 associated with ribosomal structure; IGHM, the immunoglobulin heavy constant mu transcript; LAMA2, which is involved with cell adhesion, migration, and tissue remodeling; and transcripts related to other functions such as DAB2, KREMEN1, and EVA1.

The 10 transcript classifier (Table 11) for distinguishing febrile without adenovirus versus with adenovirus infection included the interleukin-1 receptor accessory protein, 1L1RAP; two interferon induced genes, IFI27 and IFI44, which were also in the classifier for fever status; and LGALS3BP, which is involved in cell-cell and cell-matrix interactions and has been found elevated in individuals infected with the human immunodeficiency virus. Other transcripts with known functions less clearly related to adenoviral FRI or with unknown functions included ZCCHC2, ZSIG11, NOP5/NOP58, MS4A7, LY6E, and BTN3A3.

Discussion

After having rigorously assessed the RNA quality of samples processed with PAX tubes in a relatively large sample of humans with differing infection status phenotypes, we characterized and compared the transcriptomes from whole blood samples of healthy, FRI without and with adenovirus infection, and convalescent individuals, evaluated class prediction methodologies, discovered nested sets of transcripts that could optimally classify the infection status phenotypes and have begun to implicate pathways and gene functions involved in FRI.

We applied a previously reported quality control metric called the degradation factor {Auer, 2003 #26} to our RNA samples and determined that this factor correlates with quality control metrics (gapdh 3′/5′ and actin 3′/5′) present on the microarray. This degradation factor can easily be applied to microarray studies on large populations by assessing electropherogram data that is available from a bioanalyzer prior to processing microarrays and an indicator can be set to flag poor quality samples. We find that quality metrics typically used, such as the 28S/18S ratio have high variability outside the traditional standard range of 1.8 to 2.1 and poorly correlate with the quality control metrics present on the microarray.

When assessing signal to noise quality metrics, we discovered that MCH significantly affects number of present calls on the B array only, likely due to detection of low expression transcripts on the B array compared to the A array {Affymetrix, 2002 #27}. At the time of probe design, the probes on the A chip were associated with more annotation than those on the B chip. The MCH is a measure of picograms of hemoglobin per red blood cell and likely is directly related to amounts of globin mRNA in whole blood samples; prior studies have demonstrated that spiking of increasing amounts of globin mRNA transcripts into total RNA from a cell line decreases the percent present calls linearly {Affymetrix, 2003 #28}. This factor would need to be controlled in future microarray studies or globin mRNA would need to be reduced. In the present study, there was no difference of MCH among the infection status phenotypes.

During supervised analysis, we varied the fold-change cut-off threshold in addition to the P-value cut-off to optimize percent correct classification. These combined criteria select for transcripts that not only are statistically different between two groups, but also vary above a specific fold-change threshold, reducing transcripts that may represent noise. The accuracy of classification seemed to be resistant to transcript selection parameters and algorithms when the gene-expression profiles showed large consistent differences, such as between non-febrile versus febrile patients; stricter P-value and fold change cut-off levels were needed to select informative transcripts that classify the healthy and convalescent or the febrile patients to an accuracy of 87% and 91%, respectively.

Misclassified samples tended to belong to groups more likely to be heterogeneous, suggesting that the misclassification may be due to the lack of specificity of the class labels. In future studies of larger size, the convalescent group might be further sub-classified based on duration of recovery and the febrile without adenovirus group sub-classified based on specific pathogen identified. The majority of transcripts in the classifiers shown in FIG. 16 remained in the classifier 100% of the time during leave-one-out cross-validation (100% CV support). Thus, these transcripts in the classifiers are consistently different between individuals of two clinical phenotypes at the time when they present for study, as exemplified in FIG. 16a. Individuals in the FRI with adenovirus group tend to present later in illness than those without, potentially accounting for gene expression differences in the two groups. The correlation of changes in expression of these genes with infection status may also suggest that these genes are involved in the human host fever and immune responses to adenovirus infection in vivo. These transcripts consistently showed the largest fold changes between groups, suggesting that the changes in expression were at the pathway level and were unlikely to be accounted for by differences in cell concentration alone. Furthermore, there were no significant differences in cell-type concentration between the febrile without- versus with adenovirus groups. This correlation of transcripts to fever and immune responses was derived from in vivo natural infections of humans, suggesting the important role of these genes in the host response at the population level. Nested sets of transcripts resulted in similar percent-correct classifications, likely due to the fact that the expression of each transcript is not independent but correlated with other transcripts in related pathways. The discovery of transcripts with functions unrelated to immune response or with unknown functions implies that these should be further studied in infection phenotype model systems to elucidate mechanistic functions.

Our demonstration that one can predict the class of a patient with FRI due to adenovirus infection from background cases of FRI due to other etiologies support the possibility of using gene-expression in biosurveillance and pathogenesis. To our knowledge, this is the first in vivo demonstration of classification of infectious diseases via transcriptional signatures of the host. We intend to extend these findings to other respiratory pathogens, both viral and bacterial and to women, to further determine the capability of applying this technology to biodefense and infectious disease surveillance.

Numerous modifications and variations on the present invention are possible in light of the above teachings. It is, therefore, to be understood that within the scope of the accompanying claims, the invention may be practiced otherwise than as specifically described herein.

### REFERENCES

1. Cardoso, F. (2003) Breast Cancer Res 5, 303-4.

2. Fraser, C. M. (2004) in Nat Rev Genet, Vol. 5, pp. 23-33.

3. Potter, J. D. (2003) in Trends Genet, Vol. 19, pp. 690-5.

4. Simon, R. (2003) Expert Rev Mol Diagn 3, 587-95.

5. Winegarden, N. (2003) Lancet 362, 1428.

6. Affymetrix, GeneChip expression analysis technical manual. 701021 Rev. 3.

7. Shoemaker, D. D., Schadt, E. E., Armour, C. D., He, Y. D., Garrett-Engele, P., McDonagh, P. D., Loerch, P. M., Leonardson, A., Lum, P. Y., Cavet, G., Wu, L. F., Altschuler, S. J., Edwards, S., King, J., Tsang, J. S., Schimmack, G., Schelter, J. M., Koch, J., Ziman, M., Marton, M. J., Li, B., Cundiff, P., Ward, T., Castle, J., Krolewski, M., Meyer, M. R., Mao, M., Burchard, J., Kidd, M. J., Dai, H., Phillips, J. W., Linsley, P. S., Stoughton, R., Scherer, S. & Boguski, M. S. (2001) Nature 409, 922-7.

8. Affymetrix (2004), Genechip operating software version 1.2. 701439 Rev 3. http://www.affymetrix.com/support/technical/manuals.affx.

9. Lander, E. S., Linton, L. M., Birren, B., Nusbaum, C., Zody, M. C., Baldwin, J., Devon, K., Dewar, K., Doyle, M., FitzHugh, W., Funke, R., Gage, D., Harris, K., Heaford, A., Howland, J., Kann, L., Lehoczky, J., LeVine, R., McEwan, P., McKernan, K., Meldrim, J., Mesirov, J. P., Miranda, C., Morris, W., Naylor, J., Raymond, C., Rosetti, M., Santos, R., Sheridan, A., Sougnez, C., Stange-Thomann, N., Stojanovic, N., Subramanian, A., Wyman, D., Rogers, J., Sulston, J., Ainscough, R., Beck, S., Bentley, D., Burton, J., Clee, C., Carter, N., Coulson, A., Deadman, R., Deloukas, P., Dunham, A., Dunham, I., Durbin, R., French, L., Grafham, D., Gregory, S., Hubbard, T., Humphray, S., Hunt, A., Jones, M., Lloyd, C., McMurray, A., Matthews, L., Mercer, S., Milne, S., Mullikin, J. C., Mungall, A., Plumb, R., Ross, M., Shownkeen, R., Sims, S., Waterston, R. H., Wilson, R. K., Hillier, L. W., McPherson, J. D., Marra, M. A., Mardis, E. R., Fulton, L. A., Chinwalla, A. T., Pepin, K. H., Gish, W. R., Chissoe, S. L., Wendl, M. C., Delehaunty, K. D., Miner, T. L., Delehaunty, A., Kramer, J. B., Cook, L. L., Fulton, R. S., Johnson, D. L., Minx, P. J., Clifton, S. W., Hawkins, T., Branscomb, E., Predki, P., Richardson, P., Wenning, S., Slezak, T., Doggett, N., Cheng, J. F., Olsen, A., Lucas, S., Elkin, C., Uberbacher, E., Frazier, M., et al. (2001) Nature 409, 860-921.

10. Venter, J. C., Adams, M. D., Myers, E. W., Li, P. W., Mural, R. J., Sutton, G. G., Smith, H. O., Yandell, M., Evans, C. A., Holt, R. A., Gocayne, J. D., Amanatides, P., Ballew, R. M., Huson, D. H., Wortman, J. R., Zhang, Q., Kodira, C. D., Zheng, X. H., Chen, L., Skupski, M., Subramanian, G., Thomas, P. D., Zhang, J., Gabor Miklos, G. L., Nelson, C., Broder, S., Clark, A. G., Nadeau, J., McKusick, V. A., Zinder, N., Levine, A. J., Roberts, R. J., Simon, M., Slayman, C., Hunkapiller, M., Bolanos, R., Delcher, A., Dew, I., Fasulo, D., Flanigan, M., Florea, L., Halpern, A., Hannenhalli, S., Kravitz, S., Levy, S., Mobarry, C., Reinert, K., Remington, K., Abu-Threideh, J., Beasley, E., Biddick, K., Bonazzi, V., Brandon, R., Cargill, M., Chandramouliswaran, I., Charlab, R., Chaturvedi, K., Deng, Z., Di Francesco, V., Dunn, P., Eilbeck, K., Evangelista, C., Gabrielian, A. E., Gan, W., Ge, W., Gong, F., Gu, Z., Guan, P., Heiman, T. J., Higgins, M. E., Ji, R. R., Ke, Z., Ketchum, K. A., Lai, Z., Lei, Y., Li, Z., Li, J., Liang, Y., Lin, X., Lu, F., Merkulov, G. V., Milshina, N., Moore, H. M., Naik, A. K., Narayan, V. A., Neelam, B., Nusskern, D., Rusch, D. B., Salzberg, S., Shao, W., Shue, B., Sun, J., Wang, Z., Wang, A., Wang, X., Wang, J., Wei, M., Wides, R., Xiao, C., Yan, C., et al. (2001) Science 291, 1304-51.

11. Wheelan, S. J. & Boguski, M. S. (1998) Genome Res 8,168-9.

12. Nau, G. J., Richmond, J. F., Schlesinger, A., Jennings, E. G., Lander, E. S. & Young, R. A. (2002) Proc Natl Acad Sci USA 99, 1503-8.

13. Boldrick, J. C., Alizadeh, A. A., Diehn, M., Dudoit, S., Liu, C. L., Belcher, C. E., Botstein, D., Staudt, L. M., Brown, P. O. & Relman, D. A. (2002) Proc Natl Acad Sci USA 99, 972-7.

14. Chaussabel, D., Semnani, R. T., McDowell, M. A., Sacks, D., Sher, A. & Nutman, T. B. (2003) Blood 102, 672-81.

15. Cummings, C. A. & Relman, D. A. (2000) Emerg Infect Dis 6, 513-25.

16. Alizadeh, A. A., Eisen, M. B., Davis, R. E., Ma, C., Lossos, I. S., Rosenwald, A., Boldrick, J. C., Sabet, H., Tran, T., Yu, X., Powell, J. I., Yang, L., Marti, G. E., Moore, T., Hudson, J., Jr., Lu, L., Lewis, D. B., Tibshirani, R., Sherlock, G., Chan, W. C., Greiner, T. C., Weisenburger, D. D., Armitage, J. O., Warnke, R., Levy, R., Wilson, W., Grever, M. R., Byrd, J. C., Botstein, D., Brown, P. O. & Staudt, L. M. (2000) Nature 403, 503-11.

17. Alizadeh, A. A. & Staudt, L. M. (2000) Curr Opin Immunol 12, 219-25.

18. Whitney, A. R., Diehn, M., Popper, S. J., Alizadeh, A. A., Boldrick, J. C., Relman, D. A. & Brown, P. O. (2003) Proc Natl Acad Sci USA 100, 1896-901.

19. Das, R., Jett, M. & Mendis, C. (2001).

20. Affymetrix (2003), Globin Reduction Protocol: A Method for Processing Whole Blood RNA Samples for Improved Array Results http://www.affymetrix.com/support/technical/technotes/blood2_technote.pdf (Accessed September 2004).

21. Eisen, M. B., Spellman, P. T., Brown, P. O. & Botstein, D. (1998) Proc Natl Acad Sci USA 95, 14863-8.

22. Quackenbush, J. (2001) Nat Rev Genet 2, 418-27.

23. Tavazoie, S., Hughes, J. D., Campbell, M. J., Cho, R. J. & Church, G. M. (1999) Nat Genet 22, 281-5.

24. Hughes, T. R., Marton, M. J., Jones, A. R., Roberts, C. J., Stoughton, R., Armour, C. D., Bennett, H. A., Coffey, E., Dai, H., He, Y. D., Kidd, M. J., King, A. M., Meyer, M. R., Slade, D., Lum, P. Y., Stepaniants, S. B., Shoemaker, D. D., Gachotte, D., Chakraburtty, K., Simon, J., Bard, M. & Friend, S. H. (2000) Cell 102, 109-26.

25. Golub, T. R., Slonim, D. K., Tamayo, P., Huard, C., Gaasenbeek, M., Mesirov, J. P., Coller, H., Loh, M. L., Downing, J. R., Caligiuri, M. A., Bloomfield, C. D. & Lander, E. S. (1999) Science 286, 531-7.

26. West, M., Blanchette, C., Dressman, H., Huang, E., Ishida, S., Spang, R., Zuzan, H., Olson, J. A., Jr., Marks, J. R. & Nevins, J. R. (2001) Proc Natl Acad Sci USA 98, 11462-7.

27. Khan, J., Wei, J. S., Ringner, M., Saal, L. H., Ladanyi, M., Westermann, F., Berthold, F., Schwab, M., Antonescu, C. R., Peterson, C. & Meltzer, P. S. (2001) Nat Med 7, 673-9.

28. Khan, S. A., Shahani, D. T. & Agarwala, A. K. (2003) ISA Trans 42, 337-52.

29. Khan, Z. H., Mohapatra, S. K., Khodiar, P. K. & Ragu Kumar, S. N. (1998) Indian J Physiol Pharmacol 42, 321-42.

30. Muller, M. C., Merx, K., Weibetaer, A., Kreil, S., Lahaye, T., Hehlmann, R. & Hochhaus, A. (2002) Leukemia 16, 2395-9.

31. Rainen, L., Oelmueller, U., Jurgensen, S., Wyrich, R., Ballas, C., Schram, J., Herdman, C., Bankaitis-Davis, D., Nicholls, N., Trollinger, D. & Tryon, V. (2002) Clin Chem 48, 1883-90.

32. Thomson, S. A. & Wallace, M. R. (2002) Hum Genet 110, 495-502.

33. Preanalytix, PAXgene blood RNA kit handbook. http://www.preanalytix.com/pdf/RNA handbook.pdf (Accessed April 2003).

34. Jurgensen, S., Schram, J., Herdman, C., Rainen, L., Wyrich, R. & Oelmueller, U.

35. Jurgensen, S., Schram, J., Herdman, C., Rainen, L., Wyrich, R. & Oelmueller, U.

36. Preanalytix, Nuclease degradation of RNA. http://www.preanalytix.com/pdf/NucleaseDegradationofRNA.pdf (Accessed April 2003).

37. Preanalytix, Repeatability—RNA purification. http://www.preanalytix.com/pdf/relpeatability.pdf (Accessed April 2003).

38. Preanalytix, Northern blot from messenger blood RNA. http:/Hwww.preanalytix.com/pdf/NorthernBlot.pdf (Accessed April 2003).

39. Preanalytix, Long-term stability of RNA using the PAXgene™ blood RNA system. http://www.preanalytix.com/pdf/TN_Storage_PAX_{—}0702.pdf (Accessed April 2003).

40. Preanalytix, Evaluation of organic extraction of RNA from PAXgene™ blood RNA tubes. http://www.preanalytix.com/pdf/TN_OrganicExtr_PAX_{—}0702.pdf (Accessed April 2003).

41. Preanalytix, Increased Concentrations of RNA using the PAXgene™ Blood RNA System. http://www.preanalytix.com/pdf/TN_ElutionMeth_PAX_{—}0702.pdf (Accessed April 2003).

42. Preanalytix, Integrity of RNA purified from whole blood samples using the PAXgene™ system. http://www.preanalytix.com/pdf/TN_Agilent_PAX_{—}0702.pdf (Accessed April 2003).

43. Preanalytix, Purification of RNA from blood using the PAXgene™ blood RNA system following multiple freeze-thaw cycles. http://www.preanalytix.com/pdf/TN_FreezeThaw_PAX_{—}0702.pdf (Accessed April 2003).

44. Preanalytix, Effects of dry ice storage on stability of RNA purified using the PAXgene™ blood RNA system. http://www.preanalytix.com/pdf/TN_DryIceShip_PAX_{—}0702.pdf (Accessed April 2003).

45. Rainen, L., Ballas, c., Oelmueller, U., Jurgensen, S., Wyrich, R., Schram, J., Walenciak, M., Herdman, C., Paumen, M., Nicholls, N., Koga, T., Goodrich, J. & J. Vanderbeek.

46. Cole, K., Truong, V., Barone, D. & McGall, G. (2004) Nucleic Acids Res 32, e86.

47. Bartlett, J. G., Dowell, S. F., Mandell, L. A., File Jr, T. M., Musher, D. M. & Fine, M. J. (2000) Clin Infect Dis 31, 347-82.

48. Mandell, L. A., Bartlett, J. G., Dowell, S. F., File, T. M., Jr., Musher, D. M. & Whitney, C. (2003) Clin Infect Dis 37, 1405-33.

49. Summary, How The Pneumonia PORT Severity Index (PSI) is Derived

Patients are stratified into 5 severity classes by means of a 2-step process.
Step 1. Determination of whether patients meet the following criteria for class I: age <50 years, with 0 of 5 comorbid conditions (i.e., neoplastic disease, liver disease, congestive heart failure, cerebrovascular disease, and renal disease), normal or only mildly deranged vital signs, and normal mental status.
Step 2. Patients not assigned to risk class I are stratified into classes II V on the basis of points assigned for 3 demographic variables (age, sex, and nursing home residency), 5 comorbid conditions (listed above), 5 physical examination findings (pulse, 125 beats/min; respiratory rate, 30 breaths/min; systolic blood pressure, <90 mm Hg; temperature, <35° C. or 40° C.; and altered mental status), and 7 laboratory and/or radiographic findings (arterial pH, <7.35; blood urea nitrogen level, 30 mg/dL; sodium level, <130 mmol/L; glucose level, 250 mg/dL; hematocrit, <30%; hypoxemia by O2 saturation, <90% by pulse oximetry or <60 mm Hg by arterial blood gas; and pleural effusion on baseline radiograph).
For classes I III, hospitalization is usually not required. For classes IV and V, the patient will usually require hospitalization.
It should be noted that social factors, such as outpatient support mechanisms and probability of adherence to treatment, are not included in this assessment.

50. Thach, D. C., Lin, B., Walter, E., Kruzelock, R., Rowley, R. K., Tibbetts, C. & Stenger, D. A. (2003) J Immunol Methods 283, 269-79.

51. Auer, H., Lyianarachchi, S., Newsom, D., Klisovic, M. I., Marcucci, G., Kornacker, K. & Marcucci, U. (2003) Nat Genet 35, 292-3.

52. Dickinson, B.

53. Gray, G. C., Gackstetter, G. D., Kang, H. K., Graham, J. T. & Scott, K. C. (2004) Am J Prev Med 26, 443-52.

54. Patarca, R. (2001) Ann NY Acad Sci 933, 185-200.

55. Preanalytix (2003).

56. Brenner, S., Johnson, M., Bridgham, J., Golda, G., Lloyd, D. H., Johnson, D., Luo, S., McCurdy, S., Foy, M., Ewan, M., Roth, R., George, D., Eletr, S., Albrecht, G., Vermaas, E., Williams, S. R., Moon, K., Burcham, T., Pallas, M., DuBridge, R. B., Kirchner, J., Fearon, K., Mao, J. & Corcoran, K. (2000) Nat Biotechnol 18, 630-4.

57. Lin, B., Vora, G. J., Thach, D., Walter, E., Metzgar, D., Tibbetts, C. & Stenger, D. A. (2004) J Clin Microbiol 42, 3232-9.

58. Stenger, D. A., Andreadis, J. D., Vora, G. J. & Pancrazio, J. J. (2002) Curr Opin Biotechnol 13, 208-12.

59. Haab, B. B. (2001) Curr Opin Drug Discov Devel 4, 116-23.

60. Preanalytix, Product circular. PAXgene Blood RNA Tube. http://www.preanalytix.com/pdf/prodcir.pdf (Accessed April 2003).

61. Agilent (October 2002).

62. Affymetrix (2001), Microarray Suite user's guide version 5.0. 701099 Rev 1. http://www.affymetrix.com/support/technical/manuals.affx.

63. Filliben, J. J., Heckert, A. & Lipman, R. R.

64. Li, C. & Hung Wong, W. (2001) Genome Biol 2.

65. Li, C. & Wong, W. H. (2001) Proc Natl Acad Sci USA 98, 31-6.

66. Azarani, A. & Hecker, K. H. (2001) Nucleic Acids Res 29, E7.

67. Filliben, J. J. (NIST SEMATECH.

68. Affymetrix (2004), GeneChip® Expression Analysis Data Analysis Fundamentals. Part No. 701190 Rev. 4. Page 39. https://www.affymetrix.com/support/downloads/manuals/data_analysis_fundamentals_manual.pdf (accessed September 2004).

69. Affymetrix (2002), Performance and Validation of the GeneChip® Human Genome U133 Set. http://www.affymetrix.com/support/technical/technotes/hgu133_performance_technote.pdf (Accessed September 2004).

70. Hosack, D. A., Dennis, G., Jr., Sherman, B. T., Lane, H. C. & Lempicki, R. A. (2003) Genome Biol 4, R70.

71. Griffiths, M. J. et al. (2005) The Journal of Infectious Disease 191, 1599-1611.

72. Cobb, J. P. et al. (2005) Proc Natl Acad Sci USA 102, 4801-4806.

73. Rubins, K. H. et al (2004) Proc Natl Acad Sci USA 101, 15190-15195.

### Supplemental Info: List of Tables Provided in Electronic Form and Brief Description

Table 16—Performance of classifiers during cross-validation for Class Prediction for fever status (i.e., febrile versus non-febrile patients)

Table 17—Performance of classifiers during cross-validation, table of parameters for Table 16

Table 18—Composition of classifier, list of genes significant at the 0.01 level (sorted by t-value) for Class Prediction for fever status

Table 19—‘Observed v. Expected’ table of GO classes and parent classes, in list of significant genes shown in Table 18

Table 20—Performance of classifiers during cross-validation for Class Prediction for febrile with adenovirus versus without adenovirus patients

Table 21—Performance of classifiers during cross-validation, table of parameters for Table 20

Table 22—Composition of classifier, list of genes significant at the 0.01 level (sorted by t-value) for Class Prediction for rile with adenovirus versus without adenovirus patients

Table 23—‘Observed v. Expected’ table of GO classes and parent classes, in list of significant genes shown in Table 22

Table 24—Performance of classifiers during cross-validation for Class Prediction for healthy versus convalescent patients

Table 25—Performance of classifiers during cross-validation, table of parameters for Table 24

Table 26—Composition of classifier, list of genes significant at the 0.01 level (sorted by t-value) for Class Prediction for healthy versus convalescent patients

Table 27—‘Observed v. Expected’ table of GO classes and parent classes, in list of significant genes shown in Table 26

Table 28—List of genes that discriminate for fever status (i.e., febrile versus non-febrile patients)

Table 29—‘Observed v. Expected’ table of GO classes and parent classes, in list of significant genes shown in Table 28

Table 30—List of genes that discriminate for adenovirus versus without adenovirus patient

Table 31—‘Observed v. Expected’ table of GO classes and parent classes, in list of significant genes shown in Table 30

Table 32—List of genes that discriminate for healthy versus convalescent patients

Table 33—‘Observed v. Expected’ table of GO classes and parent classes, in list of significant genes shown in Table 32

1. A method for determining the gene expression profile for a subject that has been exposed to one or more infectious pathogens comprising
a) collecting a biological sample from a subject;
b) isolating RNA from said sample;
c) removing DNA contaminants from said sample;
d) spiking into said sample a normalization control;
e) synthesizing cDNA from the RNA contained in said sample;
f) in vitro transcribing cRNA from said cDNA and labeling said cRNA;
g) hybridizing said cRNA to a gene chip followed by washing, staining, and scanning; and
h) acquiring a gene expression profile from said gene chip and analyzing the gene expression profile represented by the RNA in said sample on the basis of the disease(s) said subject has been exposed to.

2. The method of claim 1, wherein said biological sample is whole blood.

3. The method of claim 1, further comprising, between (c) and (d),
concentrating and purifying said RNA.

4. The method of claim 1, further comprising, between (d) and (e),
reducing and/or eliminating globin mRNA in said sample.

5. The method of claim 4, wherein said reducing and/or eliminating globin mRNA in said sample comprises adding biotinylated globin capture oligos to said sample to bind the globin mRNA and removing the resulting bound globin mRNA by strepavidin magnetic beads leaving globinclear RNA.

6. The method of claim 5, further comprising further purifying the globinclear RNA by contacting said globinclear RNA with magnetic RNA beads.

7. The method of claim 1, further comprising, coincident with (e),
reducing and/or eliminating globin mRNA in said sample by adding PNA to said sample during said synthesizing cDNA.

8. The method of claim 1, further comprising, between (g) and (h), repeating (g) with a second gene chip which is distinct from said gene chip in (g), wherein in (h) following acquisition the data obtained from said first and second gene chips is merged.

9. A method for identifying gene expression markers for distinguishing between healthy, febrile, or convalescence in subjects that have been exposed to one or more infectious pathogens comprising
a) acquiring a gene expression profile by the method according to claim 1 for a subject that has been exposed to one or more infectious pathogens;
b) acquiring a gene expression profile by the method according to claim 1 for a subject that has recovered from exposure to said one or more infectious pathogens;
c) acquiring a gene expression profile by the method according to claim 1 for a healthy subject that has not been exposes to said one or more infectious pathogens;
d) comparing the gene expression profiles for the subjects from (a), (b), and (c) by a pairwise comparison;
e) determining the identity of the nested to minimal set(s) of genes that classify the patient phenotype as healthy, febrile, or convalescent by class prediction algorithm based on said pairwise comparison; and
f) assigning the classification of healthy, febrile, or convalescent based on gene expression profile of the minimal set of genes determined in (e).

10. A method of classifying a subject in need thereof as healthy, febrile, or convalescence, comprising
a) collecting a biological sample from said subject;
b) isolating RNA from said sample;
c) removing DNA contaminants from said sample;
d) spiking into said sample a normalization control;
e) synthesizing cDNA from the RNA contained in said sample;
f) in vitro transcribing cRNA from said cDNA and labeling said cRNA;
g) hybridizing said cRNA to a gene chip followed by washing, staining, and scanning
h) acquiring a gene expression profile from said gene chip and analyzing the gene expression profile represented by the RNA in said sample; and
i) determining the gene expression profile in said subject of the minimal set of genes that classify the patient phenotype as healthy, febrile, or convalescent determined by the method of claim 9;
j) classifying the subject in need thereof as being healthy, febrile, or convalescent by comparing the gene expression profile obtained in (i) to that of the classification assignment of healthy, febrile, or convalescent based on gene expression profile of the minimal set of genes as determined by the method of claim 9.

11. The method of claim 10, wherein said biological sample is whole blood.

12. The method of claim 10, further comprising, between (c) and (d),
concentrating and purifying said RNA.

13. The method of claim 10, further comprising, between (d) and (e),
reducing and/or eliminating globin mRNA in said sample.

14. The method of claim 13, wherein said reducing and/or eliminating globin mRNA in said sample comprises adding biotinylated globin capture oligos to said sample to bind the globin mRNA and removing the resulting bound globin mRNA by strepavidin magnetic beads leaving globinclear RNA.

15. The method of claim 14, further comprising further purifying the globinclear RNA by contacting said globinclear RNA with magnetic RNA beads.

16. The method of claim 10, further comprising, coincident with (e),
reducing and/or eliminating globin mRNA in said sample by adding PNA to said sample during said synthesizing cDNA.

17. The method of claim 10, further comprising, between (g) and (h), repeating (g) with a second gene chip which is distinct from said gene chip in (g), wherein in (h) following acquisition the data obtained from said first and second gene chips is merged.

18. The method of claim 10, wherein the minimal set of genes to distinguish non-febrile from febrile patients comprises PDCD1LG1, PLSCR1, FCGR1A, PLSCR1, FCGR1A, CEACAM1, SERPING1, TNFAIP6, ANKRD22, EPSTI1, FLJ39885, DNAPTP6, IFI35, OAS1, PRV1, STK3, GBP1, GBP1, CASP5, IFIT4, GPR105, MGC20410, cig5, LOC129607, IFI44, GBP5, C1QG, HSXIAPAF1, cig5, UPP1, PML, LAMP3, IFRG28, G1P2, C1orf29, IFI44, LIPA, OAS1, MX1, SN, HSXIAPAF1, IFIT1, OAS2, and IFI27.

19. The method of claim 10, wherein the minimal set of genes to distinguish healthy versus convalescent patients comprises RPL27, RPS7, DAB2, LAMA2, IGHM, EVA1, and KREMEN1.

20. The method of claim 10, wherein the minimal set of genes to distinguish febrile with adenovirus versus febrile without adenovirus patients comprises ILIRAP, ZCCHC2, IFI44, ZCCHC2, ZSIG11, NOP5/NOP58, LGALS3BP, MS4A7, LY6E, BTN3A3, and IF27.


SEQUENCE LISTING (5 sequences)

SEQ ID NO: 1 - DNA, 25 bp, Artificial Sequence
  Synthetic DNA
gttgctaactacgatccagatattg

SEQ ID NO: 2 - DNA, 22 bp, Artificial Sequence
  Synthetic DNA
cctggtaagtgtctgtcaatcc

SEQ ID NO: 3 - DNA, 28 bp, Artificial Sequence
  Synthetic DNA
cagtatgtggaatcaggcggtggacagc

SEQ ID NO: 4 - DNA, 20 bp, Artificial Sequence
  Synthetic DNA
tccctacgatgcagacaacg

SEQ ID NO: 5 - DNA, 21 bp, Artificial Sequence
  Synthetic DNA
agtgccatctatgctatctcc